(19) United States
(12) Patent Application Publication
(10) Pub. No.: US 2007/0111970 A1
(43) Pub. Date: May 17, 2007

(54) Inositol compounds and uses of same in the treatment of diseases characterized by abnormal protein folding or aggregation or amyloid formation, desposition, accumulation or persistence

(76) Inventors: Antonio Cruz, Toronto (CA); Linda Kurdydyk, Toronto (CA)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/580,026

(22) Filed: Oct. 13, 2006

Related U.S. Application Data
(60) Provisional application No. 60/725,634, filed on Oct. 13, 2005.

(51) Int. Cl. A61K 31/66; A61K 31/16; A61K 31/075; A61K 31/045

(52) U.S. Cl. 514/102; 514/715; 514/729; 514/616

(57) ABSTRACT

Inositol derivatives are described that are represented by the structural formula I 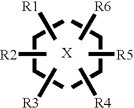
wherein X is a radical of scyllo-inositol wherein one or more of R¹, R², R³, R⁴, R⁵, and R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, halo, seleno, silyl, silyloxy, silylthio, carboxyl, carbonyl, carbamoyl, or carboxamide and the other of R¹, R², R³, R⁴, R⁵, or R⁶ are hydroxyl, or pharmaceutically acceptable salts thereof. The compounds, compositions comprising same and methods using same are described for use in the prevention and/or treatment of diseases characterized by abnormal protein folding or aggregation or amyloid formation, desposition, accumulation or persistence.

### CROSS-REFERENCE TO RELATED APPLICATIONS

This application claims the benefit of priority under 35 U.S.C. § 119(e) to U.S. Provisional Application 60/725,634 filed Oct. 13, 2005.

### FIELD OF INVENTION

The invention relates to compounds, compositions and methods for treating diseases characterized by abnormal protein folding or aggregation or amyloid formation, desposition, accumulation or persistence.

### BACKGROUND OF INVENTION

Scyllo-inositol is one of the nine known stereoisomers of hexahydroxycyclohexane (Bouveault L. Bull. La Societe Chimique Paris 1894: 11: 44-147). The compound is present in human brain in quantities estimated to from 5 to 12% that of myo-inositol (5 mM) (Michaelis T et al. NMR in Biomedicien 1993: 6: 105-109). WO 2004/075882 published Sep. 10, 2004 discloses the use of scyllo-inosital in the prevention and treatment of disorders in protein folding or aggregation, or amyloid formation, deposition, accumulation, or persistence.

### SUMMARY OF INVENTION

Broadly stated, the invention provides a method for treating a disease characterized by abnormal protein folding or aggregation or amyloid formation, deposition, accumulation or persistence in a subject comprising an isolated and pure, in particular substantially pure, compound of the formula I: 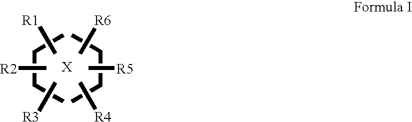
wherein X is a radical of scyllo-inositol wherein one or more of R¹, R², R³, R⁴, R⁵, and R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, halo, seleno, silyl, silyloxy, silylthio, carboxyl, carbonyl, carbamoyl, or carboxamide and the other of R¹, R², R³, R⁴, R⁵, or R⁶ are hydroxyl, or a pharmaceutically acceptable salt thereof.

The invention also provides a method for treating a disease characterized by abnormal protein folding or aggregation or amyloid formation, deposition, accumulation or persistence in a subject comprising an isolated and pure, in particular substantially pure, compound of the formula II: 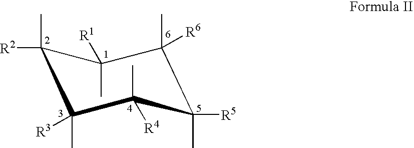
wherein one or more of R¹, R², R³, R⁴, R⁵, and R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfinyl, sulfonate, amino, imino, azido, thiol, thioalkyl, thioalkoxy, thioaryl, nitro, cyano, halo, seleno, silyl, silyloxy, silylthio, carboxyl, carbonyl, carbamoyl, or carboxamide and the other of R¹, R², R³, R⁴, R⁵, or R⁶ are hydroxyl.

In an aspect, a method is provided for treating a disease characterized by abnormal protein folding or aggregation or amyloid formation, deposition, accumulation or persistence in a subject comprising an isolated and pure, in particular substantially pure, compound of the formula I or II as defined herein with the proviso that when (a) one of R¹, R², R³, R⁴, R⁵, and R⁶ are alkyl or fluorine no more than four of the other of R¹, R², R³, R⁴, R⁵, and R⁶ are hydroxyl, (b) one of R¹, R², R³, R⁴, R⁵, and R⁶ is amino or azide no more than four of R¹, R², R³, R⁴, R⁵, and R⁶ are hydroxyl, (c) two of R¹, R², R³, R⁴, R⁵, and R⁶ are amino, no more than three of R¹, R², R³, R⁴, R⁵, and R⁶ are hydroxyl, and (d) three of R¹, R², R³, R⁴, R⁵, and R⁶ are amino, carboxy, carbamyl, sulfonyl, isoxasolyl, imidazolyl, or thazolyl the other of R¹, R², R³, R⁴, R⁵, and R⁶ cannot all be hydroxyl.

In aspects of the invention, a method is provided for treating a disease characterized by abnormal protein folding or aggregation or amyloid formation, deposition, accumulation or persistence in a subject comprising an isolated and pure, in particular substantially pure, compound of the formula I or II excluding compounds disclosed in WO 2004/075882.

The invention also provides a method for treating diseases disclosed herein in a subject comprising administering to the subject a therapeutically effective amount of one or more compound of the formula I or II, or a pharmaceutically acceptable salt thereof, or a composition comprising a compound of the formula I or II and a pharmaceutically acceptable carrier, excipient, or vehicle. In an aspect the invention provides a treatment which results in beneficial effects following treatment. The methods of the invention can be used therapeutically or can be used prophylactically in a subject susceptible to a disease disclosed herein.

In an aspect, the invention provides a method of improving memory of a healthy subject or the memory of a subject with age impaired memory by administering an effective amount of a compound of the formula I or II, or a pharmaceutically acceptable salt thereof, or a composition comprising a compound of the formula I or II and a pharmaceutically acceptable carrier, excipient, or vehicle.

The present invention further relates to a method for improving memory, especially short-term memory and other mental dysfunction associated with the aging process comprising administering an effective amount of a compound of the formula I or II, or a pharmaceutically acceptable salt thereof, or a composition comprising a compound of the formula I or II and a pharmaceutically acceptable carrier, excipient, or vehicle.

In an embodiment, a method is provided for treating a mammal in need of improved memory, wherein the mammal has no diagnosed disease, disorder, infirmity or ailment known to impair or otherwise diminish memory, comprising the step of administering to the mammal an effective memory-improving amount of a compound of the formula I or II, a pharmaceutically acceptable salt thereof, or a dietary supplement comprising a compound of the formula I or II or a nutraceutically acceptable derivative thereof.

In another aspect of the invention, a method is provided for treating in a subject a condition of the central or peripheral nervous system or systemic organ associated with a disorder in protein folding or aggregation, or amyloid formation, deposition, accumulation, or persistence, comprising administering to the subject a therapeutically effective amount of a compound of the formula I or II, or a pharmaceutically acceptable salt thereof, or a composition comprising a compound of the formula I or II and a pharmaceutically acceptable carrier, excipient, or vehicle.

In a further aspect, the invention provides a method involving administering to a subject a therapeutic compound of the formula I or II, or a pharmaceutically acceptable salt thereof, or a composition comprising a compound of the formula I or II, and a pharmaceutically acceptable carrier, excipient, or vehicle which inhibit amyloid formation, deposition, accumulation and/or persistence, and/or which cause dissolution/disruption of pre-existing amyloid. Thus, the compounds and compositions of the invention may be used for inhibiting amyloidosis in disorders in which amyloid deposition occurs.

In another aspect, the invention provides a method for treating in a subject a condition associated with an amyloid interaction that can be disrupted or dissociated with a compound of the invention comprising administering to the subject a therapeutically effective amount of a compound of the formula I or II, a pharmaceutically acceptable salt thereof, or a composition comprising a compound of the formula I or II and a pharmaceutically acceptable carrier, excipient, or vehicle.

In an aspect, the invention provides a method for preventing or inhibiting amyloid protein assembly, enhancing clearance of amyloid deposits, or slowing deposition of amyloid deposits in a subject comprising administering a therapeutically effective amount of a compound of the formula I or II a pharmaceutically acceptable salt thereof, or a composition comprising a compound of the formula I or II, and a pharmaceutically acceptable carrier, excipient, or vehicle.

In an aspect, the invention provides a method for reducing or inhibiting amyloid fibril formation, organ specific dysfunction (e.g., neurodegeneration), or cellular toxicity in a subject comprising administering to the subject a therapeutically effective amount of a compound of the formula I or II or a pharmaceutically acceptable salt thereof, or a composition comprising a compound of the formula I or II and a pharmaceutically acceptable carrier, excipient, or vehicle.

The invention has particular applications in treating a disease characterized by amyloid deposition, in particular an amyloidoses, more particularly Alzheimer's disease. Thus, the invention relates to a method of treatment comprising administering a therapeutically effective amount of one or more compound of the formula I or II, a pharmaceutically acceptable salt thereof, or a composition comprising a compound of the formula I or II and a pharmaceutically acceptable carrier, excipient, or vehicle, which upon administration to a subject with symptoms of a disease characterized by amyloid deposition, more particularly Alzheimer's disease, produces beneficial effects, preferably sustained beneficial effects. In an embodiment, beneficial effects are evidenced by one or more of the following: disruption of aggregated Aβ, increased inhibition of long term potentiation induced by Aβ oligomers and/or maintenance of synaptic function, and/or, reduced cerebral accumulation of Aβ, deposition of cerebral amyloid plaques, soluble Aβ oligomers in the brain, glial activity, inflammation, and/or cognitive decline.

In an aspect, the invention provides a method for amelioriating progression of a disease or obtaining a less severe stage of a disease in a subject suffering from such disease (e.g., Alzheimer's disease) comprising administering a therapeutically effective amount of a compound of the formula I or II, a pharmaceutically acceptable salt thereof, or a composition comprising a compound of the formula I or II, and a pharmaceutically acceptable carrier, excipient, or vehicle.

The invention relates to a method of delaying the progression of a disease (e.g., Alzheimer's disease) comprising administering a therapeutically effective amount of a compound of the formula I or II, a pharmaceutically acceptable salt thereof, or a composition comprising a compound of the formula I or II, and a pharmaceutically acceptable carrier, excipient, or vehicle.

The invention also relates to a method of increasing survival of a subject suffering from a disease comprising administering a therapeutically effective amount of a compound of the formula I or II, a pharmaceutically acceptable salt thereof, or a composition comprising a compound of the formula I or II, and a pharmaceutically acceptable carrier, excipient, or vehicle.

In an embodiment, the invention relates to a method of improving the lifespan of a subject suffering from Alzheimer's disease comprising administering a therapeutically effective amount of a compound of the formula I or II, a pharmaceutically acceptable salt thereof, or a composition comprising a compound of the formula I or II, and a pharmaceutically acceptable carrier, excipient, or vehicle.

In an aspect the invention provides a method for treating mild cognitive impairment (MCI) comprising administering a therapeutically effective amount of a compound of the formula I or II, a pharmaceutically acceptable salt thereof, or a composition comprising a compound of the formula I or II and a pharmaceutically acceptable carrier, excipient, or vehicle.

In an embodiment, the invention provides a method of reversing amyloid deposition and neuropathology after the onset of cognitive deficits and amyloid plaque neuropathology in a subject comprising administering to the subject a therapeutically effective amount of a compound of the formula I or II, a pharmaceutically acceptable salt thereof, or a composition comprising a compound of the formula I or II and a pharmaceutically acceptable carrier, excipient, or vehicle.

A compound or composition of the invention can be administered to a patient by a route effective to treat a disease disclosed herein. Exemplary routes of administration include intravenous, oral, intraperitoneal, and subcutaneous.

This invention also includes a regimen for supplementing a healthy human's diet by administering a compound of the formula I or II or a dietary supplement comprising a compound of the formula I or II or a nutraceutically acceptable derivative thereof, and an acceptable carrier, to the human. The invention further includes a regimen for supplementing a healthy human's diet by administering daily to the human a compound of the formula I or II or a nutraceutically acceptable derivative thereof.

A regimen for supplementing a human's diet is provided comprising administering to the human a supplement comprising, per gram of supplement: about 5 milligram to about 30 milligrams of one or more compound of the formula I or II or a nutraceutically acceptable derivative thereof. In an embodiment, a portion of the supplement is administered at the time of the human's morning meal, and a second portion of the supplement is administered at the time of the human's noontime meal.

In certain aspects of the invention, a compound of the formula I or II is a prodrug or comprises a carrier as described herein.

The invention also provides a compound of the formula I or II as defined herein with the proviso that when (a) one of R¹, R², R³, R⁴, R⁵, and R⁶ are alkyl or fluorine no more than 4 of the other of R¹, R², R³, R⁴, R⁵, and R⁶ are hydroxyl, (b) one of R¹, R², R³, R⁴, R⁵, and R⁶ is amino or azide no more than four of R¹, R², R³, R⁴, R⁵, and R⁶ are hydroxyl, (c) two of R¹, R², R³, R⁴, R⁵, and R⁶ are amino, no more than three of R¹, R², R³, R⁴, R⁵, and R⁶ are hydroxyl, and (d) three of R¹, R², R³, R⁴, R⁵, and R⁶ are amino, carboxy, carbamyl, sulfonyl, isoxasolyl, imidazolyl, or thazolyl the other of R¹, R², R³, R⁴, R⁵, and R⁶ cannot all be hydroxyl.

The invention also provides a compound of the formula I or II excluding compounds disclosed in WO 2004/075882.

A compound of the invention may be in the form of a prodrug that is converted in vivo to an active compound. By way of example, in a compound of the formula I or II one or more of R¹, R², R³, R⁴, R⁵, and R⁶ may be a radical group with a cleavable group that is cleaved after administration to a subject to provide an active (e.g. therapeutically active) compound, or an intermediate compound that subsequently yields the active compound. The cleavable group may be an ester that can be removed either enzymatically or non-enzymatically.

A compound of the formula I or II may optionally comprise a carrier interacting with one or more of R¹, R², R³, R⁴, R⁵, or R⁶. A carrier may include a polymer, carbohydrate, or peptide, or combinations thereof. A carrier may be substituted, for example, with one or more alkyl, halo, thiol, hydroxyl, or amino.

Compounds of the formula I or II can be incorporated in compositions for use as pharmaceuticals or dietary supplements.

In an aspect, the invention provides compositions for prevention and/or treatment of a disease disclosed herein. Thus, the invention provides a pharmaceutical composition comprising a compound of the formula I or II, in particular a therapeutically effective amount of a compound of the formula I or II for treating a disease. More particularly, the invention provides a pharmaceutical composition in a form adapted for administration to a subject to provide beneficial effects to treat a disease disclosed herein.

In another aspect, the composition is in a form such that administration to a subject suffering from a disease results in prevention, reduction and/or inhibition of Aβ fibril assembly or aggregation, Aβ toxicity, Aβ42 levels, abnormal protein folding, aggregation, amyloid formation, deposition, accumulation or persistence, and/or amyloid interactions; and/or acceleration of disassembly of preformed fibrils. A composition of the invention can be in a form that results in one or more of disruption or dissociation of aggregating Aβ; increased inhibition of long term potentiation induced by Aβ oligomers; maintenance of synaptic function; reduced cerebral accumulation of amyloid β, deposition of cerebral amyloid plaques, soluble Aβ oligomers in the brain, glial activity, inflammation, and/or cognitive decline in the subject. In addition, a composition of the invention can be in a form that results in dissolution or disruption of preformed or pre-deposited amyloid fibrils or amyloid in a subject.

In an aspect, the invention features a composition comprising a compound of the invention in a therapeutically effective amount for disrupting aggregation of Aβ, increasing reduction or inhibition of long term potentiation induced by Aβ oligomers, maintaining synaptic function, and/or reducing cerebral accumulation of amyloidβ, deposition of cerebral amyloid plaques, soluble Aβ oligomers in the brain, glial activity, inflammation, and/or cognitive decline in the subject. The composition can be in a pharmaceutically acceptable carrier, excipient, or vehicle.

The invention additionally provides a method of preparing a stable pharmaceutical composition comprising one or more compound of the formula I or II. After compositions have been prepared, they can be placed in an appropriate container and labeled for treatment of an indicated disease. For administration of a composition of the invention, such labeling would include amount, frequency, and method of administration.

The invention further provides a dietary supplement composition comprising one or more compound of the formula I or II or nutraceutically acceptable derivatives thereof. In an aspect, the invention provides a dietary supplement for mammalian consumption and particularly human consumption for the purpose of improving memory comprising a compound of the formula I or II or nutraceutically acceptable derivatives thereof. In another aspect, the invention provides a supplement comprising a compound of the formula I or II or nutraceutically acceptable derivatives thereof for slowing the deterioration of mental processes and improving memory, in particular short-term memory, of individuals who have taken the supplement.

A dietary supplement of the invention is preferably pleasant tasting, effectively absorbed into the body and provides substantial therapeutic effects.

The invention also provides methods to make commercially available pills, tablets, caplets, soft and hard gelatin capsules, lozenges, sachets, cachets, vegicaps, liquid drops, elixirs, suspensions, emulsions, solutions, syrups, aerosols (as a solid or in a liquid medium) suppositories, sterile injectable solutions, and/or sterile packaged powders, which contain a compound of the formula I or II of the invention.

In an aspect, a dietary supplement of the present invention is formulated as a beverage, but may be formulated in granule, capsule or suppository form.

In an aspect, compounds and compositions of the invention may be administered therapeutically or prophylactically to treat diseases associated with amyloid formation, aggregation or deposition. While not wishing to be bound by any particular theory, the compounds and compositions may act to ameliorate the course of a disease using without limitation one or more of the following mechanisms: preventing, reducing and/or inhibiting Aβ fibril assembly or aggregation, Aβ toxicity, Aβ42 levels, abnormal protein folding or aggregation, amyloid formation, deposition, accumulation or persistence, and/or amyloid interactions; inhibiting or reducing neurodegeneration or cellular toxicity induced by Aβ; accelerating disassembly of preformed fibrils; disrupting or dissociating aggregating Aβ; increasing inhibition of long term potentiation induced by Aβ oligomers; maintaining synaptic function; enhancing clearance of Aβ from the brain; increasing degradation of Aβ; and/or, reducing cerebral accumulation of amyloid β, deposition of cerebral amyloid plaques, soluble Aβ oligomers in the brain, glial activity, inflammation, and/or cognitive decline.

The invention also contemplates the use of at least one compound of the formula I or II or a composition comprising same for the preparation of a medicament for treating diseases. The invention additionally provides uses of a pharmaceutical composition of the invention in the preparation of medicaments for the prevention and/or treatment of diseases. The medicament may be in a form for consumption by a subject such as a pill, tablet, caplet, soft and hard gelatin capsule, lozenge, sachet, cachet, vegicap, liquid drop, elixir, suspension, emulsion, solution, syrup, aerosol (as a solid or in a liquid medium) suppository, sterile injectable solution, and/or sterile packaged powder for inhibition of amyloid formation, deposition, accumulation, and/or persistence, regardless of its clinical setting.

The invention also provides a kit comprising one or more compound or a composition of the invention. In an aspect, the invention provides a kit for preventing and/or treating a disease, containing a composition comprising one or more compound, a container, and instructions for use. The composition of the kit can further comprise a pharmaceutically acceptable carrier, excipient, or vehicle. In an aspect, the invention provides a method of promoting sales of a composition or kit of the invention comprising the public distribution of information that administration of the composition or kit is associated with treatment or prophylaxis of a disease disclosed herein.

In another aspect, the invention relates to a pharmaceutical composition for treating a disease characterized by abnormal protein folding or aggregation or amyloid formation, deposition, accumulation or persistence in a subject comprising a therapeutically effective amount of a compound of formula III, 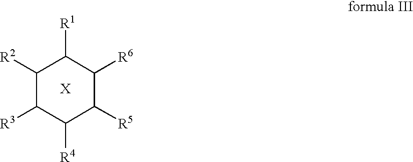
wherein X is a cyclohexane ring, where at least one of R¹, R², R³, R⁴, R⁵, and R⁶ is independently selected from hydrogen, C₁-C₆ alkyl, C₂-C₆ alkenyl, C₂-C₆ alkynyl, C₁-C₆ alkoxy, C₂-C₆ alkenyloxy, C₃-C₁₀ cycloalkyl, C₄-C₁₀ cycloalkenyl, C₃-C₁₀ cycloalkoxy, C₆-C₁₀ aryl, C₆-C₁₀ aryloxy, C₆-C₁₀ aryl-C₁-C₃ alkoxy, C₆-C₁₀ aroyl, C₆-C₁₀ heteroaryl, C₃-C₁₀ heterocyclic, C₁-C₆ acyl, C₁-C₆ acyloxy, —NH₂, —NHR⁷, —NR⁷R⁸, ═NR⁷, —S(O)₀₋₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆ alkenyl, C₂-C₆ alkynyl, C₃-C₁₀ cycloalkyl, C₄-C₁₀ cycloalkenyl, C₆-C₁₀ aryl, C₆-C₁₀ aryl C₁-C₃alkyl, C₆-C₁₀ heteroaryl and C₃-C₁₀ heterocyclic; and at least one of the remainder of R¹, R², R³, R⁴, R⁵, or R⁶ is hydroxyl, or a pharmaceutically acceptable salt thereof, and a pharmaceutically acceptable carrier, excipient, or vehicle.

In yet another aspect, the invention relates to a pharmaceutical composition comprising a therapeutically effective amount of a compound of formula IV, 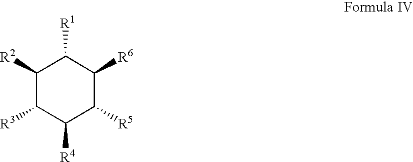
wherein R¹, R², R³, R⁴, R⁵, and R⁶ are defined as for Formula III, or a pharmaceutically acceptable salt thereof and a pharmaceutically acceptable carrier, excipient, or vehicle.

In another aspect, the invention relates to the pharmaceutical composition comprising a compound of Formula III or IV, where R² is hydroxyl; and R¹, R³, R⁴, R⁵, and R⁶ are independently selected from C₁-C₆ alkyl, C₂-C₆ alkenyl, C₂-C₆ alkynyl, C₁-C₆ alkoxy, C₂-C₆ alkenyloxy, C₃-C₁₀ cycloalkyl, C₄-C₁₀ cycloalkenyl, C₃-C₁₀ cycloalkoxy, C₆-C₁₀ aryl, C₆-C₁₀ aryloxy, C₆-C₁₀ aryl-C₁-C₃ alkoxy, C₆-C₁₀ aroyl, C₆-C₁₀ heteroaryl, C₃-C₁₀ heterocyclic, C₁-C₆ acyl, C₁-C₆ acyloxy, hydroxyl, —NH₂, —NHR⁷, —NR⁷R⁸—, ═NR⁷, —S(O)₀₋₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆ alkenyl, C₂-C₆ alkynyl, C₃-C₁₀ cycloalkyl, C₄-C₁₀ cycloalkenyl, C₆-C₁₀ aryl, C₆-C₁₀aryl C₁-C₃alkyl, C₆-C₁₀ heteroaryl and C₃-C₁₀ heterocyclic; provided that R¹, R², R³, R⁴, R⁵, and R⁶ are not all hydroxyl; and a pharmaceutically acceptable carrier, excipient, or vehicle.

In yet another aspect, the invention relates to the pharmaceutical composition comprising a compound of Formula III or IV, where R² is hydroxyl; one of R¹, R³, R⁴, R⁵, and R⁶ is hydroxyl; and four of R¹, R³, R⁴, R⁵, and R⁶ are independently selected from C₁-C₆ alkyl, C₂-C₆ alkenyl, C₂-C₆ alkynyl, C₁-C₆ alkoxy, C₂-C₆ alkenyloxy, C₃-C₁₀ cycloalkyl, C₄-C₁₀ cycloalkenyl, C₃-C₁₀ cycloalkoxy, C₆-C₁₀ aryl, C₆-C₁₀ aryloxy, C₆-C₁₀ aryl-C₁-C₃ alkoxy, C₆-C₁₀ aroyl, C₆-C₁₀ heteroaryl, C₃-C₁₀ heterocyclic, C₁-C₆ acyl, C₁-C₆ acyloxy, —NH₂, —NHR⁷, —NR⁷R⁸—, ══NR⁷, —S(O)₀₋₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and S(O)₂NR⁷R⁸ wherein R⁷ and R⁸ are independently selected from C₁-C₆ alkyl, C₂-C₆ alkenyl, C₂-C₆ alkynyl, C₃-C₁₀ cycloalkyl, C₄-C₁₀ cycloalkenyl, C₆-C₁₀ aryl, C₆-C₁₀aryl C₁-C₃ alkyl, C₆-C₁₀ heteroaryl and C₃-C₁₀ heterocyclic; and a pharmaceutically acceptable carrier, excipient, or vehicle.

In another aspect, the invention relates to the pharmaceutical composition comprising a compound of Formula III or IV, where R² is hydroxyl; two of R¹, R³, R⁴, R⁵, and R⁶ are hydroxyl; and three of R¹, R³, R⁴, R⁵, and R⁶ are independently selected from C₁-C₆ alkyl, C₂-C₆ alkenyl, C₂-C₆ alkynyl, C₁-C₆ alkoxy, C₂-C₆ alkenyloxy, C₃-C₁₀ cycloalkyl, C₄-C₁₀ cycloalkenyl, C₃-C₁₀ cycloalkoxy, C₆-C₁₀ aryl, C₆-C₁₀ aryloxy, C₆-C₁₀ aryl-C₁-C₃ alkoxy, C₆-C₁₀ aroyl, C₆-C₁₀ heteroaryl, C₃-C₁₀ heterocyclic, C₁-C₆ acyl, C₁-C₆ acyloxy, —NH₂, —NHR⁷, —NR⁷R⁸—, ═NR⁷, —S(O)₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆ alkenyl, C₂-C₆ alkynyl, C₃-C₁₀ cycloalkyl, C₄-C₁₀ cycloalkenyl, C₆-C₁₀ aryl, C₆-C₁₀aryl C₁-C₃alkyl, C₆-C₁₀ heteroaryl and C₃-C₁₀ heterocyclic; and a pharmaceutically acceptable carrier, excipient, or vehicle.

In another aspect, the invention relates to the pharmaceutical composition comprising a compound of Formula III or IV, where R² is hydroxyl; three of R¹, R³, R⁴, R⁵, and R⁶ is hydroxyl; and two of R¹, R³, R⁴, R⁵, and R⁶ are independently selected from C₁-C₆ alkyl, C₂-C₆ alkenyl, C₂-C₆ alkynyl, C₁-C₆ alkoxy, C₂-C₆ alkenyloxy, C₃-C₁₀ cycloalkyl, C₄-C₁₀ cycloalkenyl, C₃-C₁₀ cycloalkoxy, C₆-C₁₀ aryl, C₆-C₁₀ aryloxy, C₆-C₁₀ aryl-C₁-C₃ alkoxy, C₆-C₁₀ aroyl, C₆-C₁₀ heteroaryl, C₃-C₁₀ heterocyclic, C₁-C₆ acyl, C₁-C₆ acyloxy, —NH₂, —NHR⁷, —NR⁷R⁸—, ═NR⁷, —S(O)₀₋₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆ alkenyl, C₂-C₆ alkynyl, C₃-C₁₀ cycloalkyl, C₄-C₁₀ cycloalkenyl, C₆-C₁₀ aryl, C₆-C₁₀aryl C₁-C₃alkyl, C₆-C₁₀ heteroaryl and C₃-C₁₀ heterocyclic; and a pharmaceutically acceptable carrier, excipient, or vehicle.

In yet another aspect, the invention relates to the pharmaceutical composition comprising a compound of Formula III or IV, where R² is hydroxyl; four of R¹, R³, R⁴, R⁵, and R⁶ are hydroxyl; and one of R¹, R³, R⁴, R⁵, and R⁶ are independently selected from C₁-C₆ alkyl, C₂-C₆ alkenyl, C₂-C₆ alkynyl, C₁-C₆ alkoxy, C₂-C₆ alkenyloxy, C₃-C₁₀ cycloalkyl, C₄-C₁₀ cycloalkenyl, C₃-C₁₀ cycloalkoxy, C₆-C₁₀ aryl, C₆-C₁₀ aryloxy, C₆-C₁₀ aryl-C₁-C₃ alkoxy, C₆-C₁₀ aroyl, C₆-C₁₀ heteroaryl, C₃-C₁₀ heterocyclic, C₁-C₆ acyl, C₁-C₆ acyloxy, —NH₂, —NHR⁷, —NR⁷R⁸—, ═NR⁷, —S(O)₀₋₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and S(O)₂NR⁷R⁸ wherein R⁷ and R⁸ are independently selected from C₁-C₆ alkyl, C₂-C₆ alkenyl, C₂-C₆ alkynyl, C₃-C₁₀ cycloalkyl, C₄-C₁₀ cycloalkenyl, C₆-C₁₀ aryl, C₆-C₁₀aryl C₁-C₃ alkyl, C₆-C₁₀ heteroaryl and C₃-C₁₀ heterocyclic; and a pharmaceutically acceptable carrier, excipient, or vehicle.

In another aspect, the invention relates to the pharmaceutical composition comprising a compound of Formula III or IV, wherein one of R¹, R³, R⁴, R⁵, and R⁶ is C₁-C₆ alkyl, C₁-C₆ alkoxy, C₁-C₆ acyl, halo, oxo, ═NR⁷, —NHC(O)R⁷, —C(O)NH₂, —C(O)NHR⁷, —C(O)NR⁷R⁸, CO₂R⁷, or —SO₂R⁷, wherein R⁷R⁸ are as defined above; and no more than four of the remainder of R¹, R², R³, R⁴, R⁵, and R⁶ are hydroxyl; and a pharmaceutically acceptable carrier, excipient, or vehicle.

In another aspect, the invention relates to the pharmaceutical composition comprising a compound of Formula III or IV, wherein two of R¹, R³, R⁴, R⁵, and R⁶ are C₁-C₆ alkyl, C₁-C₆ alkoxy, C₁-C₆ acyl, halo, oxo, ═NR⁷, —NHC(O)R⁷, —C(O)NH₂, —C(O)NHR⁷, —C(O)NR⁷R⁸, CO₂R⁷, or —SO₂R⁷, wherein R⁷R⁸ are as defined above; and no more than three of R¹, R², R³, R⁴, R⁵, and R⁶ are hydroxyl; and a pharmaceutically acceptable carrier, excipient, or vehicle.

In yet another aspect, the invention relates to the pharmaceutical composition comprising a compound of Formula III or IV, wherein three of R¹, R³, R⁴, R⁵, and R⁶ are C₁-C₆ alky, C₁-C₆ alkoxy, C₁-C₆ alkyl, halo, oxo, ═NR⁷, —NHC(O)R⁷, —C(O)NH₂, —C(O)NHR⁷, —C(O)NR⁷R⁸, CO₂R⁷, or —SO₂R⁷, wherein R⁷R⁸ are as defined above; and no more than two of R¹, R², R³, R⁴, R⁵, and R⁶ are hydroxyl; and a pharmaceutically acceptable carrier, excipient, or vehicle.

In another aspect, the invention relates to the pharmaceutical composition comprising a compound of Formula III or IV, wherein four of R¹, R², R³, R⁴, R⁵, or R⁶ are hydroxyl; and one of R¹, R², R³, R⁴, R⁵, or R⁶ is each independently selected from the group CH₃, OCH₃, NO₂, CF₃, OCF₃, F, Cl, Br, I and CN; and a pharmaceutically acceptable carrier, excipient, or vehicle.

In still another aspect, the invention relates to the pharmaceutical composition comprising a compound of Formula III or IV, wherein five of R¹, R², R³, R⁴, R⁵, or R⁶ are hydroxyl; and one of R¹, R², R³, R⁴, R⁵, or R⁶ is selected from CH₃, OCH₃, NO₂, CF₃, OCF₃, F, Cl, Br, I and CN; and a pharmaceutically acceptable carrier, excipient, or vehicle.

In still another aspect, the invention relates to the pharmaceutical composition comprising a compound of Formula III or IV, wherein two, three, four or five of R¹, R², R³, R⁴, R⁵, or R⁶ are hydroxyl; at least one of R¹, R², R³, R⁴, R⁵, or R⁶ is optionally substituted alkoxy; and the remainder of R¹, R², R³, R⁴, R⁵, or R⁶ if any are independently selected from C₁-C₆ alkyl, C₂-C₆ alkenyl, C₂-C₆ alkynyl, C₁-C₆ alkoxy, C₂-C₆ alkenyloxy, C₃-C₁₀ cycloalkyl, C₁-C₆ acyl, C₁-C₆ acyloxy, hydroxyl, —NH₂, —NHR⁷, —NR⁷R⁸—, ═NR⁷, —S(O)₀₋₂R⁷, —SH, nitro, cyano, halo, haloalkyl, haloalkoxy, hydroxyalkyl, —CO₂R⁷, oxo, —PO₃H—NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆ alkenyl, C₂-C₆ alkynyl, C₃-C₁₀ cycloalkyl, C₄-C₁₀ cycloalkenyl, C₆-C₁₀ aryl, C₆-C₁₀aryl C₁-C₃ alkyl, C₆-C₁₀ heteroaryl and C₃-C₁₀ heterocyclic; and a pharmaceutically acceptable carrier, excipient, or vehicle.

In still another aspect, the invention relates to the pharmaceutical composition comprising a compound of Formula III or IV, wherein five of R¹, R², R³, R⁴, R⁵, or R⁶ are hydroxyl; and one of R¹, R², R³, R⁴, R⁵, or R⁶ is C₁-C₆ alkoxy; and a pharmaceutically acceptable carrier, excipient, or vehicle, for example at least one of R¹, R², R³, R⁴, R⁵, or R⁶ is methoxy.

In still another aspect, the invention relates to a pharmaceutical composition comprising a compound of Formula IV, wherein two, three, or four of R², R³, R⁴, R⁵, or R⁶ are hydroxyl; R¹ is optionally substituted alkoxy; and the remainder of R², R³, R⁴, R⁵, or R⁶ are independently selected from C₁-C₆ alkyl, C₂-C₆ alkenyl, C₂-C₆ alkynyl, C₁-C₆ alkoxy, C₂-C₆ alkenyloxy, C₃-C₁₀ cycloalkyl, C₁-C₆ acyl, C₁-C₆ acyloxy, hydroxyl, —NH₂, —NHR⁷, —NR⁷R⁸—, ═NR⁷, —S(O)₀₋₂R⁷, —SH, nitro, cyano, halo, haloalkyl, haloalkoxy, hydroxyalkyl, —CO₂R⁷, oxo, —PO₃H—NHC(O)R⁷, —C(O)NH₂, —C(O)NHR⁷, —C(O)NR⁷R⁸, —NHS(O)₂R⁷, —S(O)₂NH₂, S(O)₂NHR⁷, and —S(O)₂NR⁷R⁸ wherein R⁷ and R⁸ are independently selected from C₁-C₆ alkyl, C₂-C₆ alkenyl, C₂-C₆ alkynyl, C₃-C₁₀ cycloalkyl, C₄-C₁₀ cycloalkenyl, C₆-C₁₀ aryl, C₆-C₁₀aryl C₁-C₃ alkyl, C₆-C₁₀ heteroaryl and C₃-C₁₀ heterocyclic; and a pharmaceutically acceptable carrier, excipient, or vehicle.

In still another aspect, the invention relates to the pharmaceutical composition comprising a compound of Formula IV, wherein R¹ is C₁-C₆ alkoxy; and R², R³, R⁴, R⁵, and R⁶ are hydroxyl; and a pharmaceutically acceptable carrier, excipient, or vehicle, for example R¹ is methoxy.

In another aspect, the invention relates to a pharmaceutical composition wherein the compound is methyl-scyllo-inositol 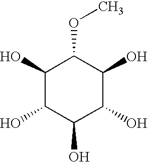
and a pharmaceutically acceptable carrier, excipient, or vehicle.

In another aspect, the invention relates to the pharmaceutical composition comprising a compound of Formula III or IV, wherein two, three, four or five of R¹, R², R³, R⁴, R⁵, or R⁶ are hydroxyl; at least one of R¹, R², R³, R⁴, R⁵, or R⁶ is halo; and the remainder of R¹, R², R³, R⁴, R⁵, or R⁶, if any, are independently C₁-C₆ alkyl, C₂-C₆ alkenyl, C₂-C₆ alkynyl, C₁-C₆ alkoxy, C₂-C₆ alkenyloxy, C₃-C₁₀ cycloalkyl, C₁-C₆ acyl, C₁-C₆ acyloxy, —NH₂, —NHR⁷, —NR⁷R⁸—, ═NR⁷, —S(O)₀₋₂R⁷, —SH, nitro, cyano, halo, haloalkyl, haloalkoxy, hydroxyalkyl, —CO₂R⁷, oxo, —PO₃H—NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆ alkenyl, C₂-C₆ alkynyl, C₃-C₁₀ cycloalkyl, C₄-C₁₀ cycloalkenyl, C₆-C₁₀ aryl, C₆-C₁₀aryl C₁-C₃ alkyl, C₆-C₁₀ heteroaryl and C₃-C₁₀ heterocyclic; and a pharmaceutically acceptable carrier, excipient, or vehicle.

In still another aspect, the invention relates to the pharmaceutical composition comprising a compound of Formula III or IV, wherein four of R¹, R², R³, R⁴, R⁵, or R⁶ are hydroxyl; one of R¹, R², R³, R⁴, R⁵, or R⁶ is halo; and one of R¹, R², R³, R⁴, R⁵, or R⁶ is selected from C₁-C₆ alkyl, C₂-C₆ alkenyl, C₂-C₆ alkynyl, C₁-C₆ alkoxy, C₂-C₆ alkenyloxy, C₃-C₁₀ cycloalkyl, C₁-C₆ acyl, C₁-C₆ acyloxy, hydroxyl, —NH₂, —NHR⁷, —NR⁷R⁸—, ═NR⁷, —S(O)₀₋₂R⁷, —SH, nitro, cyano, halo, haloalkyl, haloalkoxy, hydroxyalkyl, —Si(R⁷)₃, —CO₂R⁷, oxo, —PO₃H—NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆ alkenyl, C₂-C₆ alkynyl, C₃-C₁₀ cycloalkyl, C₄-C₁₀ cycloalkenyl, C₆-C₁₀ aryl, C₆-C₁₀aryl C₁-C₃ alkyl, C₆-C₁₀ heteroaryl and C₃-C₁₀ heterocyclic, and at least one of R¹, R², R³, R⁴, R⁵, or R⁶ is halo; and a pharmaceutically acceptable carrier, excipient, or vehicle.

In yet another aspect, the invention relates to the pharmaceutical composition comprising a compound of Formula III or IV, wherein five of R¹, R², R³, R⁴, R⁵, or R⁶ are hydroxyl; and one of R¹, R², R³, R⁴, R⁵, or R⁶ is halo; and a pharmaceutically acceptable carrier, excipient, or vehicle, for example the invention relates to the pharmaceutical composition comprising a compound of Formula IV, wherein R², R³, R⁴, R⁵, or R⁶ are hydroxyl, and R¹ is halo; preferably the halo is fluoro, chloro or bromo.

In another aspect, the invention relates to a pharmaceutical composition wherein the compound is 1-chloro-1-deoxy-scyllo-inositol: 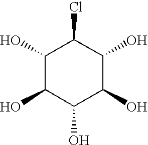
and a pharmaceutically acceptable carrier, excipient, or vehicle.

In another aspect, the invention relates to the pharmaceutical composition comprising a compound of Formula III or IV, or methyl-scyllo-inositol or 1-chloro-1-deoxy-scyllo-inositol for use in the treatment of a disease that is characterized by amyloid deposition, for examples for the use in the treatment of Alzheimer's disease.

In another aspect, the invention relates to a method for preventing, reducing and/or inhibiting in a subject Aβ fibril assembly or aggregation, Aβ toxicity, Aβ42 levels, abnormal protein folding or aggregation, amyloid formation, deposition, accumulation or persistence, and/or amyloid interactions comprising administering a therapeutically effective amount of the pharmaceutical composition comprising a compound of Formula III or IV, or methyl-scyllo-inositol or 1-chloro-1-deoxy-scyllo-inositol.

In another aspect, the invention relates to a method for increasing degradation of Aβ and/or reducing cerebral accumulation of amyloid β, deposition of cerebral amyloid plaques, soluble Aβ oligomers in the brain, glial activity, inflammation, and/or cognitive decline comprising administering a therapeutically effective amount of the pharmaceutical composition comprising a compound of Formula III or IV, or methyl-scyllo-inositol or 1-chloro-1-deoxy-scyllo-inositol.

In another aspect, the invention relates to a method for treating in a subject a condition of the central or peripheral nervous system or systemic organ associated with a disorder in protein folding or aggregation, or amyloid formation, deposition, accumulation, or persistence, comprising administering to the subject a therapeutically effective amount of a pharmaceutical composition comprising a compound of Formula III or IV, or methyl-scyllo-inositol or 1-chloro-1-deoxy-scyllo-inositol.

In another aspect, the invention relates to a method for preventing or inhibiting amyloid protein assembly, enhancing clearance of amyloid deposits, or slowing deposition of amyloid deposits in a subject comprising administering to the subject a therapeutically effective amount of a pharmaceutical composition comprising a compound of Formula III or IV, or methyl-scyllo-inositol or 1-chloro-1-deoxy-scyllo-inositol.

In another aspect, the invention relates to a method of delaying the progression of Alzheimer's disease in a subject comprising administering to the subject a therapeutically effective amount of a pharmaceutical composition comprising a compound of Formula III or IV, or methyl-scyllo-inositol or 1-chloro-1-deoxy-scyllo-inositol.

In another aspect, the invention relates to a method for treating mild cognitive impairment (MCI) in a subject comprising administering to the subject a therapeutically effective amount of a pharmaceutical composition comprising a compound of Formula III or IV, or methyl-scyllo-inositol or 1-chloro-1-deoxy-scyllo-inositol.

In another aspect, the invention relates to a regimen for supplementing a human's diet comprising administering a composition comprising a compound of Formula III, IV, methyl-scyllo-inositol or 1-chloro-1-deoxy-scyllo-inositol or a dietary supplement comprising a composition comprising a compound of Formula III or IV, or methyl-scyllo-inositol or 1-chloro-1-deoxy-scyllo-inositol, and an acceptable carrier, to the human; in some embodiments the regiment is administered daily to the human and in other embodiments the regiment is administered in an amount from about 5 milligrams to about 30 milligrams.

In another aspect, the invention relates to a kit comprising the composition containing at least one compound of Formula III or IV, or methyl-scyllo-inositol or 1-chloro-1-deoxy-scyllo-inositol for preventing and/or treating a disease characterized by abnormal protein folding or aggregation or amyloid formation, deposition, accumulation or persistence, a container, and instructions for use; and in some embodiments the instructions provide information for treating Alzheimer's disease.

These and other aspects, features, and advantages of the present invention should be apparent to those skilled in the art from the following detailed description.

### DETAILED DESCRIPTION OF EMBODIMENTS

For convenience, certain terms employed in the specification, examples, and appended claims are collected here.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made. Further, it is to be understood that “a,” “an,” and “the” include plural referents unless the content clearly dictates otherwise. Thus, for example, reference to a composition comprising “a compound” includes a mixture of two or more compounds.

The terms “administering” and “administration” refer to the process by which a therapeutically effective amount of a compound or composition contemplated herein is delivered to a subject for prevention and/or treatment purposes. Compositions are administered in accordance with good medical practices taking into account the subject's clinical condition, the site and method of administration, dosage, patient age, sex, body weight, and other factors known to physicians.

The term “treating” refers to reversing, alleviating, or inhibiting the progress of a disease, or one or more symptoms of such disease, to which such term applies. Treating includes the management and care of a subject at diagnosis or later. A treatment may be either performed in an acute or chronic way. Depending on the condition of the subject, the term also refers to preventing a disease, and includes preventing the onset of a disease, or preventing the symptoms associated with a disease. The term also refers to reducing the severity of a disease or symptoms associated with such disease prior to affliction with the disease. Such prevention or reduction of the severity of a disease prior to affliction refers to administration of a compound or composition of the present invention to a subject that is not at the time of administration afflicted with the disease. “Preventing” also refers to preventing the recurrence of a disease or of one or more symptoms associated with such disease. An objective of treatment is to combat the disease and includes administration of the active compounds to prevent or delay the onset of the symptoms or complications, or alleviating the symptoms or complications, or eliminating or partially eliminating the condition and/or disease. The terms “treatment” and “therapeutically,” refer to the act of treating, as “treating” is defined above.

The terms “subject”, “individual”, or “patient” are used interchangeably herein and refer to an animal including a warm-blooded animal such as a mammal. Mammal includes without limitation any members of the Mammalia. In general, the terms refer to a human. The terms also include domestic animals bred for food or as pets, including horses, cows, sheep, poultry, fish, pigs, cats, dogs, and zoo animals, goats, apes (e.g. gorilla or chimpanzee), and rodents such as rats and mice. Typical subjects for treatment include persons afflicted with or suspected of having or being pre-disposed to a disease disclosed herein, or persons susceptible to, suffering from or that have suffered a disease described herein. A subject may or may not have a genetic predisposition for a disease disclosed herein such as Alzheimer's disease. In particular aspects, a subject shows signs of cognitive deficits and amyloid plaque neuropathology. In embodiments of the invention the subjects are suspectible to, or suffer from Alzheimer's disease.

As utilized herein, the term “healthy subject” means a subject, in particular a mammal, having no diagnosed disease, disorder, infirmity, or ailment known to impair or otherwise diminish memory.

A “beneficial effect” refers to an effect of a compound of the invention or composition thereof in certain aspects of the invention, including favorable pharmacological and/or therapeutic effects, and improved biological activity. In aspects of the invention, the beneficial effects include without limitation prevention, reduction or inhibition of Aβ fibril assembly or aggregation, Aβ toxicity, Aβ42 levels, abnormal protein folding, aggregation, amyloid formation, deposition, accumulation or persistence, and/or amyloid lipid interactions, and/or acceleration of disassembly of preformed fibrils. In particular embodiments of the invention, the beneficial effects include but are not limited to the following: disruption of aggregated Aβ; increased inhibition of long term potentiation induced by Aβ oligomers; maintenance of synaptic function; inhibition of Aβ-induced progressive cognitive decline and cerebral amyloid plaque pathology; improved cognition; increased lifespan; reduced cerebral accumulation of Aβ; reduced deposition of cerebral amyloid plaques; reduced soluble Aβ oligomers (e.g. Aβ42) in the brain; reduced glial activity; reduced inflammation; and/or cognitive decline. In some aspects, a beneficial effect is a favourable characteristic of a composition/formulation of the invention includes enhanced stability, a longer half life, and/or enhanced uptake and transport across the blood brain barrier.

The beneficial effect may be a statistically significant effect in terms of statistical analysis of an effect of a compound of the invention versus the effects without the compound or an inositol compound that is not within the scope of the invention (e.g. myo-inositol or unmodified scyllo-inositol). “Statistically significant” or “significantly different” effects or levels may represent levels that are higher or lower than a standard. In embodiments of the invention, the difference may be 1.5, 2, 3, 4, 5, or 6 times higher or lower compared with the effect obtained without a compound of the invention.

The term “pharmaceutically acceptable carrier, excipient, or vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbants that may be needed in order to prepare a particular composition. Examples of carriers etc. include but are not limited to saline, buffered saline, dextrose, water, glycerol, ethanol, and combinations thereof. The use of such media and agents for an active substance is well known in the art.

“Pharmaceutically acceptable salt(s),” means a salt that is pharmaceutically acceptable and has the desired pharmacological properties. By pharmaceutically acceptable salts is meant those salts which are suitable for use in contact with the tissues of a subject or patient without undue toxicity, irritation, allergic response and the like, and are commensurate with a reasonable benefit/risk ratio. Pharmaceutically acceptable salts are described for example, in S. M. Berge, et al., J. Pharmaceutical Sciences, 1977, 66:1. Suitable salts include salts that may be formed where acidic protons in the compounds are capable of reacting with inorganic or organic bases. Suitable inorganic salts include those formed with alkali metals, e.g. sodium and potassium, magnesium, calcium, and aluminum. Suitable organic salts include those formed with organic bases such as the amine bases, e.g. ethanolamine, diethanolamine, triethanolamine, tromethamine, N-methylglucamine, and the like. Suitable salts also include acid addition salts formed with inorganic acids (e.g. hydrochloric and hydrobromic acids) and organic acids (e.g. acetic acid, citric acid, maleic acid, and the alkane- and arene-sulfonic acids such as methanesulfonic acid and benezenesulfonic acid). When there are two acidic groups present, a pharmaceutically acceptable salt may be a mono-acid-mono-salt or a di-salt; and similarly where there are more than two acidic groups present, some or all of such groups can be salified.

“Therapeutically effective amount” relates to the amount or dose of an active compound or composition of the invention that will lead to one or more desired effects, in particular, one or more beneficial effects. A therapeutically effective amount of a substance can vary according to factors such as the disease state, age, sex, and weight of the subject, and the ability of the substance to elicit a desired response in the subject. A dosage regimen may be adjusted to provide the optimum therapeutic response (e.g. sustained beneficial effects). For example, several divided doses may be administered daily or the dose may be proportionally reduced as indicated by the exigencies of the therapeutic situation.

As used “nutraceutically acceptable derivative” refers to a derivative or substitute for the stated chemical species that operates in a similar manner to produce the intended effect, and is structurally similar and physiologically compatible. Examples of substitutes include without limitation salts, esters, hydrates, or complexes of the stated chemical. The substitute could also be a precursor or prodrug to the stated chemical, which subsequently undergoes a reaction in vivo to yield the stated chemical or a substitute thereof.

The term “pure” in general means better than 90%, 92%, 93%, 94%, 95%, 96%, 97%, 98% or 99% pure, and “substantially pure” means a compound synthesized such that the compound, as made as available for consideration into a composition or therapeutic dosage of the invention, has only those impurities that can not readily nor reasonably be removed by conventional purification processes.

A “polymer” as used herein refers to molecules comprising two or more monomer subunits that may be identical repeating subunits or different repeating subunits. A monomer generally comprises a simple structure, low-molecular weight molecule containing carbon. Polymers can be optionally substituted. Examples of polymers which can be used in the present invention are vinyl, acryl, styrene, carbohydrate derived polymers, polyethylene glycol (PEG), polyoxyethylene, polymethylene glycol, polytrimethylene glycols, polyvinylpyrrolidone, polyoxyethylene-polyoxypropylene block polymers, and copolymers, salts, and derivatives thereof. In particular aspects of the invention, the polymer is poly(2-acrylamido-2-methyl-1-propanesulfonic acid); poly(2-acrylamido-2-methyl,-1-propanesulfonic acid-coacrylonitrile, poly(2-acrylamido-2-methyl-1-propanesulfonic acid-co-styrene), poly(vinylsulfonic acid); poly(sodium 4-styrenesulfonic acid); and sulfates and sulfonates derived therefrom; poly(acrylic acid), poly(methylacrylate), poly(methyl methacrylate), and poly(vinyl alcohol).

A “carbohydrate” as used herein refers to a polyhydroxyaldehyde, or polyhydroxyketone and derivatives thereof. The simplest carbohydrates are monosaccharides, which are small straight-chain aldehydes and ketones with many hydroxyl groups added, usually one on each carbon except the functional group. Examples of monosaccharides include erythrose, arabinose, allose, altrose, glucose, mannose, threose, xylose, gulose, idose, galactose, talose, aldohexose, fructose, ketohexose, ribose, and aldopentose. Other carbohydrates are composed of monosaccharide units, including disaccharides, oligosaccharides, or polysaccharides, depending on the number of monosaccharide units. Disaccharides are composed of two monosaccharide units joined by a covalent glycosidic bond. Examples of disaccharides are sucrose, lactose, and maltose. Oligosaccharides and polysaccharides are composed of longer chains of monosaccharide units bound together by glycosidic bonds. Oligosaccharides generally contain between 3 and 9 monosaccharide units and polysaccharides contain greater than 10 monosaccharide units. A carbohydrate group may be substituted at one two, three or four positions, other than the position of linkage to a compound of the formula I or II. For example, a carbohydrate may be substituted with one or more alkyl, amino, nitro, halo, thiol, carboxyl, or hydroxyl groups, which are optionally substituted. Illustrative substituted carbohydrates are glucosamine or galactosamine.

In aspects of the invention, the carbohydrate is a sugar, in particular a hexose or pentose and may be an aldose or a ketose. A sugar may be a member of the D or L series and can include amino sugars, deoxy sugars, and their uronic acid derivatives. In embodiments of the invention where the carbohydrate is a hexose, the hexose is selected from the group consisting of glucose, galactose, or mannose, or substituted hexose sugar residues such as an amino sugar residue such as hexosamine, galactosamine, glucosamine, in particular D-glucosamine (2-amino-2-doexy-D-glucose) or D-galactosamine (2-amino-2-deoxy-D-galactose). Suitable pentose sugars include arabinose, fucose, and ribose.

A sugar residue may be linked to a compound of the formula I or II from a 1,1 linkage, 1,2 linkage, 1,4 linkage, 1,5 linkage, or 1,6 linkage. A linkage may be via an oxygen atom of a compound of the formula I or II. An oxygen atom can be replaced one or more times by —CH₂— or —S— groups.

The term “carbohydrate” also includes glycoproteins such as lectins (e.g. concanavalin A, wheat germ agglutinin, peanutagglutinin, seromucoid, and orosomucoid) and glycolipids such as cerebroside and ganglioside.

A “peptide” for use as a carrier in the practice of the present invention includes one, two, three, four, or five or more amino acids covalently linked through a peptide bond. A peptide can comprise one or more naturally occurring amino acids, and analogs, derivatives, and congeners thereof. A peptide can be modified to increase its stability, bioavailability, solubility, etc. “Peptide analogue” and “peptide derivative” as used herein include molecules which mimic the chemical structure of a peptide and retain the functional properties of the peptide. In aspects of the invention the carrier is an amino acid such as alanine, glycine, proline, methionine, serine, threonine, histidine, or asparagine. In other aspects the carrier is a peptide such as alanyl-alanyl, prolyl-methionyl, or glycyl-glycyl. In still other aspects, the carrier is a polypeptide such as albumin, antitrypsin, macroglobulin, haptoglobin, caeruloplasm, transferring, α- or β-lipoprotein, β- or γ-globulin or fibrinogen.

Approaches to designing peptide analogues, derivatives and mimetics are known in the art. For example, see Farmer, P. S. in Drug Design (E. J. Ariens, ed.) Academic Press, New York, 1980, vol. 10, pp. 119-143; Ball, J. B. and Alewood, P. F. (1990) J. Mol. Recognition 3:55; Morgan, B. A. and Gainor, J. A. (1989) Ann. Rep. Med. Chem. 24:243; and Freidinger, R. M. (1989) Trends Pharmacol. Sci. 10:270. See also Sawyer, T. K. (1995) “Peptidomimetic Design and Chemical Approaches to Peptide Metabolism” in Taylor, M. D. and Amidon, G. L. (eds.) Peptide-Based Drug Design: Controlling Transport and Metabolism, Chapter 17; Smith, A. B. 3rd, et al. (1995) J. Am. Chem. Soc. 117:11113-11123; Smith, A. B. 3rd, et al. (1994) J. Am. Chem. Soc. 116:9947-9962; and Hirschman, R., et al. (1993) J. Am. Chem. Soc. 115:12550-12568.

Examples of peptide analogues, derivatives and peptidomimetics include peptides substituted with one or more benzodiazepine molecules (see e.g., James, G. L. et al. (1993) Science 260:1937-1942), peptides with methylated amide linkages and “retro-inverso” peptides (see U.S. Pat. No. 4,522,752 by Sisto).

Examples of peptide derivatives include peptides in which an amino acid side chain, the peptide backbone, or the amino- or carboxy-terminus has been derivatized (e.g., peptidic compounds with methylated amide linkages).

The term mimetic, and in particular, peptidomimetic, is intended to include isosteres. The term “isostere” refers to a chemical structure that can be substituted for a second chemical structure because the steric conformation of the first structure fits a binding site specific for the second structure. The term specifically includes peptide back-bone modifications (i.e., amide bond mimetics) well known to those skilled in the art. Such modifications include modifications of the amide nitrogen, the alpha-carbon, amide carbonyl, complete replacement of the amide bond, extensions, deletions or backbone crosslinks. Other examples of isosteres include peptides substituted with one or more benzodiazepine molecules (see e.g., James, G. L. et al. (1993) Science 260:1937-1942)

Other possible modifications include an N-alkyl (or aryl) substitution ([CONR]), backbone crosslinking to construct lactams and other cyclic structures, substitution of all D-amino acids for all L-amino acids within the compound (“inverso” compounds) or retro-inverso amino acid incorporation ([NHCO]). By “inverso” is meant replacing L-amino acids of a sequence with D-amino acids, and by “retro-inverso” or “enantio-retro” is meant reversing the sequence of the amino acids (“retro”) and replacing the L-amino acids with D-amino acids. For example, if the parent peptide is Thr-Ala-Tyr, the retro modified form is Tyr-Ala-Thr, the inverso form is thr-ala-tyr, and the retro-inverso form is tyr-ala-thr (lower case letters refer to D-amino acids). Compared to the parent peptide, a retro-inverso peptide has a reversed backbone while retaining substantially the original spatial conformation of the side chains, resulting in a retro-inverso isomer with a topology that closely resembles the parent peptide. See Goodman et al. “Perspectives in Peptide Chemistry” pp. 283-294 (1981). See also U.S. Pat. No. 4,522,752 by Sisto for further description of “retro-inverso” peptides.

A peptide can be attached to a compound of the invention through a functional group on the side chain of certain amino acids (e.g. serine) or other suitable functional groups. In an embodiment of the invention the carrier may comprise four or more amino acids with groups attached to three or more of the amino acids through functional groups on side chains. In another embodiment, the carrier is one amino acid, in particular a sulfonate derivative of an amino acid, for example cysteic acid.

“Alkyl”, either alone or within other terms such as “thioalkyl” and “arylalkyl” means a monovalent, saturated hydrocarbon radical which may be a straight chain (i.e. linear) or a branched chain. In certain aspects of the invention, an alkyl radical comprises from about 1 to 20 carbon atoms, preferably from about 1 to 10, 1 to 8 or 3 to 8, more preferably about 3 to 6 carbon atoms. Examples of alkyl radicals include methyl, ethyl, n-propyl, n-butyl, n-pentyl, n-hexyl, isopropyl, isobutyl, isopentyl, amyl, sec-butyl, tert-butyl, tert-pentyl, n-heptyl, n-octyl, n-nonyl, n-decyl, undecyl, n-dodecyl, n-tetradecyl, pentadecyl, n-hexadecyl, heptadecyl, n-octadecyl, nonadecyl, eicosyl, dosyl, n-tetracosyl, and the like, along with branched variations thereof. In certain embodiments of the invention an alkyl radical is a C₁-C₆ lower alkyl comprising or selected from the group consisting of methyl, ethyl, n-propyl, n-butyl, n-pentyl, n-hexyl, isopropyl, isobutyl, isopentyl, amyl, tributyl, sec-butyl, tert-butyl, tert-pentyl, and n-hexyl. An alkyl radical may be optionally substituted with substituents at positions that do not significantly interfere with the preparation of compounds of the formula I or II and that do not significantly reduce the efficacy of the compounds. An alkyl radical may be optionally substituted with groups as defined herein. In certain aspects, an alkyl radical is substituted with one to five substituents including halo, lower-alkoxy, hydroxyl, cyano, nitro, thio, amino, substituted amino, carboxyl, sulfonyl, sulfenyl, sulfinyl, sulfate, sulfoxide, substituted carboxyl, halogenated lower alkyl (e.g. CF₃), halogenated lower alkoxy, hydroxycarbonyl, lower alkoxycarbonyl, lower alkylcarbonyloxy, lower alkylcarbonylamino, and aryl (e.g., phenylmethyl (i.e. benzyl)).

The term “alkenyl” refers to an unsaturated, acyclic branched or straight-chain hydrocarbon radical comprising at least one double bond. Alkenyl radicals may contain from about 2 to 10 carbon atoms, preferably from about 3 to 8 carbon atoms and more preferably about 3 to 6 carbon atoms. Examples of suitable alkenyl radicals include ethenyl, propenyl such as prop-1-en-1-yl, prop-1-en-2-yl, prop-2-en-1-yl (allyl), prop-2-en-2-yl, buten-1-yl, but-1-en-2-yl, 2-methyl-prop-1-en-1-yl, but-2-en-1-yl, but-2-en-2-yl, buta-1,3-dien-1-yl, buta-1,3-dien-2-yl, hexen-1-yl, 3-hydroxyhexen-1-yl, hepten-1-yl, and octen-1-yl, and the like. An alkenyl radical may be optionally substituted similar to alkyl. In certain aspects, an alkenyl radical is substituted with one to five substituents including halo, lower alkoxy, hydroxyl, cyano, nitro, thio, amino, substituted amino, carboxyl, sulfonyl, sulfenyl, sulfinyl, sulfate, sulfoxide, substituted carboxyl, halogenated lower alkyl, halogenated lower alkoxy, hydroxycarbonyl, lower alkoxycarbonyl, lower alkylcarbonyloxy, lower alkylcarbonylamino, and aryl.

The term “alkynyl” refers to an unsaturated, branched or straight-chain hydrocarbon radical comprising one or more triple bonds. Alkynyl radicals may contain about 1 to 20, 1 to 15, or 2-10 carbon atoms, preferably about 3 to 8 carbon atoms and more preferably about 3 to 6 carbon atoms. Examples of suitable alkynyl radicals include ethynyl, such as prop-1-yn-1-yl, prop-2-yn-1-yl, butynyls such as but-1-yn-1-yl, but-1-yn-3-yl, but-3-yn-1-yl, pentynyls such as pentyn-1-yl, pentyn-2-yl, 4-methoxypentyn-2-yl, 3-methylbutyn-1-yl, hexynyls such as hexyn-1-yl, hexyn-2-yl, hexyn-3-yl, and 3,3-dimethylbutyn-1-yl radicals and the like. This radical may be optionally substituted similar to alkyl. In certain aspects, an alkynyl radical is substituted with one to five substituents including halo, lower alkoxy, hydroxyl, cyano, nitro, thio, amino, substituted amino, carboxyl, sulfonyl, sulfenyl, sulfinyl, sulfate, sulfoxide, substituted carboxyl, halogenated lower alkyl, halogenated lower alkoxy, hydroxycarbonyl, lower alkoxycarbonyl, lower alkylcarbonyloxy, lower alkylcarbonylamino, and aryl.

The term “alkylene” refers to a linear or branched radical having from about 1 to 10 carbon atoms and having attachment points for two or more covalent bonds. Examples of such radicals are methylene, ethylene, ethylidene, methylethylene, and isopropylidene.

The term “alkenylene” refers to a linear or branched radical having from about 2 to 10 carbon atoms, at least one double bond, and having attachment points for two or more covalent bonds. Examples of such radicals are 1,1-vinylidene (CH₂═C), 1,2-vinylidene (—CH═CH—), and 1,4-butadienyl (—CH═CH—CH═CH—).

The term “halo” refers to a halogen such as fluorine, chlorine, bromine or iodine atoms.

The term “hydroxyl” or “hydroxy” refers to a single —OH group.

The term “cyano” refers to a carbon radical having three of four covalent bonds shared by a nitrogen atom, in particular —CN.

The term “alkoxy” refers to a linear or branched oxy-containing radical having an alkyl portion of one to about ten carbon atoms, such as a methoxy radical, which may be substituted. Particular alkoxy radicals are “lower alkoxy” radicals having about 1 to 6 carbon atoms. An alkoxy having about 1-6 carbon atoms includes a C₁-C₆ alkyl-O— radical wherein C₁-C₆ alkyl has the meaning set out herein. Illustrative examples of alkoxy radicals include without limitation methoxy, ethoxy, propoxy, butoxy, isopropoxy and tert-butoxy. An “alkoxy” radical may optionally be further substituted with one or more substitutents disclosed herein including alkyl atoms to provide “alkylalkoxy” radicals; halo atoms, such as fluoro, chloro or bromo, to provide “haloalkoxy” radicals (e.g. fluoromethoxy, chloromethoxy, trifluoromethoxy, difluoromethoxy, trifluoroethoxy, fluoroethoxy, tetrafluoroethoxy, pentafluoroethoxy, and fluoropropoxy) and “haloalkoxyalkyl” radicals (e.g. fluoromethoxymethyl, chloromethoxyethyl, trifluoromethoxymethyl, difluoromethoxyethyl, and trifluoroethoxymethyl).

The term “alkenyloxy” refers to linear or branched oxy-containing radicals having an alkenyl portion of about 2 to 10 ten carbon atoms, such as an ethenyloxy or propenyloxy radical. Particular alkenyloxy radicals are “lower alkenyloxy” radicals having about 2 to 6 carbon atoms. Examples of alkenyloxy radicals include ethenyloxy, propenyloxy, butenyloxy, and isopropenyloxy alkyls. An “alkenyloxy” radical may be substituted with one or more substitutents disclosed herein including halo atoms, such as fluoro, chloro or bromo, to provide “haloalkenyloxy” radicals (e.g. trifluoroethenyloxy, fluoroethenyloxy, difluoroethenyloxy, and fluoropropenyloxy).

The term “cycloalkyl” refers to radicals having from about 3 to 15 carbon atoms and containing one, two, three, or four rings wherein such rings may be attached in a pendant manner or may be fused, in particular cyclopropyl, cyclobutyl, cyclopentyl, cyclohexyl, cycloheptyl, cyclooctyl, cyclononyl, adamantyl, and the like. In certain aspects of the invention the cycloalkyl radicals are “lower cycloalkyl” radicals having from about 3 to 8 carbon atoms, in particular cyclopropyl, cyclobutyl, cyclopentyl, cyclohexyl and cycloheptyl. The term “cycloalkyl” also embraces radicals where cycloalkyl radicals are fused with aryl radicals or heterocyclyl radicals. A cycloalkyl radical may be optionally substituted with groups as disclosed herein. In certain aspects, an alkenyl radical is substituted with one to five substituents including halo, lower alkoxy, hydroxyl, cyano, nitro, thio, amino, substituted amino, carboxyl, sulfonyl, sulfenyl, sulfinyl, sulfate, sulfoxide, substituted carboxyl, halogenated lower alkyl, halogenated lower alkoxy, hydroxycarbonyl, lower alkoxycarbonyl, lower alkylcarbonyloxy, lower alkylcarbonylamino, and aryl.

The term “cycloalkenyl” refers to radicals comprising about 2 to 15 carbon atoms, one or more carbon-carbon double bonds, and one, two, three, or four rings wherein such rings may be attached in a pendant manner or may be fused. In certain aspects of the invention the cycloalkenyl radicals are “lower cycloalkenyl” radicals having three to seven carbon atoms, in particular cyclobutenyl, cyclopentenyl, cyclohexenyl and cycloheptenyl. A cycloalkenyl radical may be optionally substituted with groups as disclosed herein.

The term “cycloalkynyl” refers to cyclic alkynyl groups.

The term “cycloalkoxy” refers to cycloalkyl radicals attached to an oxy radical. Examples of cycloalkoxy radicals include cyclohexoxy and cyclopentoxy. A cycloalkoxy radical may be optionally substituted with groups as disclosed herein.

The term “aryl”, alone or in combination, refers to a carbocyclic aromatic system containing one, two or three rings wherein such rings may be attached together in a pendant manner or may be fused. The term “fused” means that a second ring is present (i.e, attached or formed) by having two adjacent atoms in common or shared with the first ring. The term “aryl” includes without limitation aromatic radicals such as phenyl, naphthyl, indenyl, benzocyclooctenyl, benzocycloheptenyl, pentalenyl, azulenyl, tetrahydronaphthyl, indanyl, biphenyl, acephthylenyl, fluorenyl, phenalenyl, phenanthrenyl, and anthracenyl. An aryl radical may be optionally substituted with groups as disclosed herein, for example hydroxyl, alkyl, carbonyl, carboxyl, thiol, amino, and/or halo. Examples of substituted aryl radicals include phenyl, chlorophenyl, and amino phenyl.

The term “aryloxy” refers to aryl radicals, as defined above, attached to an oxygen atom. Exemplary aryloxy groups include napthyloxy, quinolyloxy, isoquinolizinyloxy, and the like.

The term “arylalkoxy,” as used herein, refers to an aryl group attached to an alkoxy group. Representative examples of arylalkoxy include, but are not limited to, 2-phenylethoxy, 3-naphth-2-ylpropoxy, and 5-phenylpentyloxy.

The term “aroyl” refers to aryl radicals, as defined above, attached to a carbonyl radical as defined herein, including without limitation benzoyl and toluoyl. An aroyl radical may be optionally substituted with groups as disclosed herein.

The term “heteroaryl” refers to fully unsaturated heteroatom-containing ring-shaped aromatic radicals having from 5 to 15 ring members selected from carbon, nitrogen, sulfur and oxygen, wherein at least one ring atom is a heteroatom. A heteroaryl radical may contain one, two or three rings and the rings may be attached in a pendant manner or may be fused. Examples of “heteroaryl” radicals, include without limitation, an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyrimidyl, pyrazinyl, pyridazinyl, triazolyl, tetrazolyl and the like; an unsaturated condensed heterocyclic group containing 1 to 5 nitrogen atoms, in particular, indolyl, isoindolyl, indolizinyl, benzimidazolyl, quinolyl, isoquinolyl, indazolyl, benzotriazolyl, tetrazolopyridazinyl and the like; an unsaturated 3 to 6-membered heteromonocyclic group containing an oxygen atom, in particular, 2-furyl, 3-furyl, and the like; an unsaturated 5 to 6-membered heteromonocyclic group containing a sulfur atom, in particular, 2-thienyl, 3-thienyl, and the like; unsaturated 5 to 6-membered heteromonocyclic group containing 1 to 2 oxygen atoms and 1 to 3 nitrogen atoms, in particular, oxazolyl, isoxazolyl, and oxadiazolyl; an unsaturated condensed heterocyclic group containing 1 to 2 oxygen atoms and 1 to 3 nitrogen atoms, in particular benzoxazolyl, benzoxadiazolyl and the like; an unsaturated 5 to 6-membered heteromonocyclic group containing 1 to 2 sulfur atoms and 1 to 3 nitrogen atoms, for example, thiazolyl, thiadiazolyl and the like; an unsaturated condensed heterocyclic group containing 1 to 2 sulfur atoms and 1 to 3 nitrogen atoms such as benzothiazolyl, benzothiadiazolyl and the like. The term also includes radicals where heterocyclic radicals are fused with aryl radicals, in particular bicyclic radicals such as benzofuran, benzothiophene, and the like. A heteroaryl radical may be optionally substituted with groups as disclosed hereinfor example hydroxyl, alkyl, carbonyl, carboxyl, thiol, amino, and/or halo.

The term “heterocyclic” refers to saturated and partially saturated heteroatom-containing ring-shaped radicals having from about 5 to 15 ring members selected from carbon, nitrogen, sulfur and oxygen, wherein at least one ring atom is a heteroatom. A heterocylic radical may contain one, two or three rings wherein such rings may be attached in a pendant manner or may be fused. Examples of saturated heterocyclic radicals include without limitation a saturated 3 to 6-membered heteromonocylic group containing 1 to 4 nitrogen atoms [e.g. pyrrolidinyl, imidazolidinyl, piperidino, and piperazinyl]; a saturated 3 to 6-membered heteromonocyclic group containing 1 to 2 oxygen atoms and 1 to 3 nitrogen atoms [e.g. morpholinyl]; and, a saturated 3 to 6-membered heteromonocyclic group containing 1 to 2 sulfur atoms and 1 to 3 nitrogen atoms [e.g., thiazolidinyl] etc. Examples of partially saturated heterocyclyl radicals include without limitation dihydrothiophene, dihydropyran, dihydrofuran and dihydrothiazole. Illustrative heterocyclic radicals include without limitation 2-pyrrolinyl, 3-pyrrolinyl, pyrrolindinyl, 1,3-dioxolanyl, 2H-pyranyl, 4H-pyranyl, piperidinyl, 1,4-dioxanyl, morpholinyl, 1,4-dithianyl, thiomorpholinyl, and the like. A heterocyclic radical may be optionally substituted with groups as disclosed herein, for example hydroxyl, alkyl, carbonyl, carboxyl, thiol, amino, and/or halo

The term “sulfate”, used alone or linked to other terms, is art recognized and includes a group that can be represented by the formula: 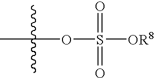
wherein R⁸ is an electron pair, hydrogen, alkyl, alkenyl, alkynyl, cycloalkyl, cycloalkenyl, cycloalkynyl, aryl, heterocyclic, carbohydrate, peptide or peptide derivative.

The term “sulfonyl”, used alone or linked to other terms such as alkylsulfonyl or arylsulfonyl, refers to the divalent radicals —SO₂—. In aspects of the invention where one or more of R¹, R³, R⁴, R⁵, or R⁶ is a sulfonyl group, the sulfonyl group may be attached to a substituted or unsubstituted alkyl group, alkenyl group, alkynyl group, aryl group, cycloalkyl group, cycloalkenyl group, cycloalkynyl group, heterocyclic group, carbohydrate, peptide, or peptide derivative.

The term “sulfonate” is art recognized and includes a group represented by the formula: 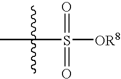
wherein R⁸ is an electron pair, hydrogen, alkyl, cycloalkyl, aryl, alkenyl, alkynyl, cycloalkenyl, cycloalkynyl, heterocyclic, carbohydrate, peptide, or peptide derivative

Examples of sulfonated alkyl groups include ethyl sulfuric acid, ethanesulfonic acid, 2-aminoethan-1-ol sulfuric acid, 1-propanesulfonic acid, 2-propanesulfonic acid, 1,2-diethanedisulfonic acid, 1,2-ethanediol disulfuric acid, 1,3-propanedisulfonic acid, 1-propanol sulfuric acid, 1,3-propanediol disulfuric acid, 1-butanesulfonic acid, 1,4-butanediol disulfuric acid, 1,2-ethanediol disulfuric acid, 3-amino-1-propanesulfonic acid, 3-hydroxypropanesulfonic acid sulfate, 1,4-butanesulfonic acid, 1,4-butanediol monosulfuric acid, 1-pentanesulfonic acid, 1,5-pentanedisulfonic acid, 1,5-pentanediol sulfuric acid, 4-heptanesulfonic acid, 1,3,5-heptanetriol trisulfate, 2-hydroxymethyl-1,3-propanediol trisulfate, 2-hydroxymethyl-2-methyl-1,3-propanediol trisulfate, 1,3,5,7-heptanetetraol tetrasulfate, 1,3,5,7,9-nonane pentasulfate, 1-decanesulfonic acid, and pharmaceutically acceptable salts thereof.

Examples of cycloalkyl sulfonated groups include 1,3-cyclohexanediol disulfate, 1,3,5-heptanetriol trisulfate.

Examples of aryl sulfonated groups include 1,3-benzenedisulfonic acid, 2,5-dimethoxy-1,4-benzenedisulfonic acid, 4-amino-3-hydroxy-1-naphthalenesulfonic acid, 3,4-diamino-1-naphthalenesulfonic acid, and pharmaceutically acceptable salts thereof.

Examples of a heterocyclic sulfonated compound include 3-(N-morpholino)propanesulfonic acid and tetrahydrothiophene-1,1-dioxide-3,4-disulfonic acid, and pharmaceutically acceptable salts thereof.

Examples of a sulfonated carbohydrate are sucrose octasulfonate, 5-deoxy-1,2-O-isopropylidene-α-D-xylofuranose-5-sulfonic acid or an alkali earth metal salt thereof, methyl-α-D-glucopyranoside 2,3-disulfate, methyl 4, —O-benzylidene-α-D-glucopyranoside 2,3-disulfate, 2,3,4,3′,4′-sucrose pentasulfate, 1,3:4,6-di-O-benzylidene-D-mannitol 2,5-disulfate, D-mannitol 2,5-disulfate, 2,5-di-O-benzyl-D-mannitol tetrasulfate, and pharmaceutically acceptable salts thereof.

The term “sulfinyl”, used alone or linked to other terms such as alkylsulfinyl (i.e. —S(O)-alkyl) or arylsulfinyl, refers to the divalent radicals —S(O)—.

The term “sulfoxide” refers to the radical —S═O.

The term “sulfenyl” or “sulfanyl” refers to the radical SR⁹ wherein R⁹ is not hydrogen. R⁹ may be alkyl, alkenyl, alkynyl, cycloalkyl, aryl, silyl, heterocyclic, heteroaryl, carbonyl, or carboxyl.

The term “amino”, alone or in combination, refers to a radical where a nitrogen atom (N) is bonded to three substituents being any combination of hydrogen, hydroxyl, alkyl, cycloalkyl, alkenyl, alkynyl, aryl or silyl with the general chemical formula —NR¹⁰R¹¹ where R¹⁰ and R¹¹ can be any combination of hydrogen, hydroxyl, alkyl, cycloalkyl, alkenyl, alkynyl, aryl, silyl, heteroaryl, or heterocyclic which may or may not be substituted. Optionally one substituent on the nitrogen atom may be a hydroxyl group (—OH) to provide an amine known as a hydroxylamine. Illustrative examples of amino groups are amino (—NH₂), alkylamino, acylamino, cycloamino, acycloalkylamino, arylamino, arylalkylamino, and lower alkylsilylamino, in particular methylamino, ethylamino, dimethylamino, 2-propylamino, butylamino, isobutylamino, cyclopropylamino, benzylamino, allylamino, hydroxylamino, cyclohexylamino, piperidine, benzylamino, diphenylmethylamino, tritylamino, trimethylsilylamino, and dimethyl-tert.-butylsilylamino.

The term “thiol” means —SH.

The term “thioalkyl”, alone or in combination, refers to a chemical functional group where a sulfur atom (S) is bonded to an alkyl, which may be substituted. Examples of thioalkyl groups are thiomethyl, thioethyl, and thiopropyl.

The term “thioaryl”, alone or in combination, refers to a chemical functional group where a sulfur atom (S) is bonded to an aryl group with the general chemical formula —SR¹² where R¹² is an aryl group which may be substituted. Illustrative examples of thioaryl groups and substituted thioaryl groups are thiophenyl, para-chlorothiophenyl, thiobenzyl, 4-methoxy-thiophenyl, 4-nitro-thiophenyl, and para-nitrothiobenzyl.

The term “thioalkoxy”, alone or in combination, refers to a chemical functional group where a sulfur atom (S) is bonded to an alkoxy group with the general chemical formula —SR¹³ where R¹³ is an alkoxy group which may be substituted. In aspects of the invention a “thioalkoxy group” has 1-6 carbon atoms and refers to a —S—(O)—C₁-C₆ alkyl group wherein C₁-C₆ alkyl have the meaning as defined above. Illustrative examples of a straight or branched thioalkoxy group or radical having from 1 to 6 carbon atoms, also known as a C₁-C₆ thioalkoxy, include thiomethoxy and thioethoxy.

The term “carbonyl” refers to a carbon radical having two of the four covalent bonds shared with an oxygen atom.

The term “carboxyl”, alone or in combination, refers to —C(O)OR¹⁴— wherein R¹⁴ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted. In aspects of the invention, the carboxyl groups are in an esterified form and may contain as an esterifying group lower alkyl groups. In particular aspects of the invention, the carboxyl groups are methoxycarbonyl, butoxycarbonyl, tert.alkoxycarbonyl such as tert.butoxycarbonyl, arylmethyoxycarbonyl having one ortwo aryl radicals including without limitation phenyl optionally substituted by, for example, lower alkyl, lower alkoxy, hydroxyl, halo, and/or nitro, such as benzyloxycarbonyl, methoxybenxyloxycarbonyl, diphenylmethoxycarbonyl, 2-bromoethoxycarbonyl, 2-iodoethoxycarbonyltert.butylcarbonyl, 4-nitrobenzyloxycarbonyl, diphenylmethoxy-carbonyl, benzhydroxycarbonyl, di-(4-methoxyphenyl-methoxycarbonyl, 2-bromoethoxycarbonyl, 2-iodoethoxycarbonyl, 2-trimethylsilylethoxycarbonyl, or 2-triphenylsilylethoxycarbonyl. Additional carboxyl groups in esterified form are silyloxycarbonyl groups including organic silyloxycarbonyl. The silicon substituent in such compounds may be substituted with lower alkyl (e.g. methyl), alkoxy (e.g. methoxy), and/or halo (e.g. chlorine). Examples of silicon substituents include trimethylsilyl and dimethyltert.butylsilyl.

The term “carboxylic ester”, alone or in combination, refers to —C(O)OR²¹ where R²¹ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic. In particular embodiments, —C(O)OR²¹ is an ester or an amino acid derivative.

The term “carboxamide”, alone or in combination, refers to amino, monoalkylamino, dialkylamino, monocycloalkylamino, alkylcycloalkylamino, and dicycloalkylamino radicals, or a heterocyclyl group containing a nitrogen atom, attached to one of two unshared bonds in a carbonyl group.

The term “carbamoyl” refers to a functional group —N(CO)O—, wherein either the nitrogen or the oxygen atom may attach to the substituted cyclohexane radical and the other is mono or di(in the case of the nitrogen atom only) substituted with alkyl, alkenyl, alkynyl, aryl, heterocyclyl, heteroaryl, cycloalkyl, or the nitrogen atom may be a member of a heterocylic ring.

The term “nitro” means —NO₂—.

The term “acyl”, alone or in combination, means a carbonyl or thiocarbonyl group bonded to a radical selected from, for example, optionally substituted, hydrido, alkyl (e.g. haloalkyl), alkenyl, alkynyl, alkoxy (“acyloxy” including acetyloxy, butyryloxy, iso-valeryloxy, phenylacetyloxy, benzoyloxy, p-methoxybenzoyloxy, and substituted acyloxy such as alkoxyalkyl and haloalkoxy), aryl, halo, heterocyclyl, heteroaryl, sulfinyl (e.g. alkylsulfinylalkyl), sulfonyl (e.g. alkylsulfonylalkyl), cycloalkyl, cycloalkenyl, thioalkyl, thioaryl, amino (e.g alkylamino or dialkylamino), and aralkoxy. Illustrative examples of “acyl” radicals are formyl, acetyl, 2-chloroacetyl, 2-bromacetyl, benzoyl, trifluoroacetyl, phthaloyl, malonyl, nicotinyl, and the like.

The terms used herein for radicals including “alkyl”, “alkoxy”, “alkenyl”, “alkynyl”, “hydroxyl” etc. refer to both unsubstituted and substituted radicals. The term “substituted,” as used herein, means that any one or more moiety on a designated atom (e.g., hydrogen) is replaced with a selection from a group disclosed herein, provided that the designated atom's normal valency is not exceeded, and that the substitution results in a stable compound. Combinations of substituents and/or radicals are permissible only if such combinations result in stable compounds. “Stable compound” refers to a compound that is sufficiently robust to survive isolation to a useful degree of purity from a reaction mixture, and formulation into an efficacious therapeutic agent.

A radical in a compound of the formula I may be substituted with one or more substituents apparent to a person skilled in the art including without limitation alkyl, alkenyl, alkynyl, alkanoyl, alkylene, alkenylene, hydroxyalkyl, haloalkyl, haloalkylene, haloalkenyl, alkoxy, alkenyloxy, alkenyloxyalkyl, alkoxyalkyl, aryl, alkylaryl, haloalkoxy, haloalkenyloxy, heterocyclic, heteroaryl, sulfonyl, sulfenyl, alkylsulfonyl, sulfinyl, alkylsulfinyl, aralkyl, heteroaralkyl, cycloalkyl, cycloalkenyl, cycloalkoxy, cycloalkenyloxy, amino, oxy, halo, azido, thio, cyano, hydroxyl, phosphonato, phosphinato, thioalkyl, alkylamino, arylamino, arylsulfonyl, alkylcarbonyl, arylcarbonyl, heteroarylcarbonyl, heteroarylsulfinyl, heteroarylsulfonyl, heteroarylamino, heteroaryloxy, heteroaryloxylalkyl, arylacetamidoyl, aryloxy, aroyl, aralkanoyl, aralkoxy, aryloxyalkyl, haloaryloxyalkyl, heteroaroyl, heteroaralkanoyl, heteroaralkoxy, heteroaralkoxyalkyl, thioaryl, arylthioalkyl, alkoxyalkyl, and acyl groups.

A “disease(s)” refers to one or more pathological symptoms or syndromes for which a cyclohexanehexol, especially a scyllo-inositol compound, provide a therapeutic effect. A “disease” includes a condition characterized by abnormal protein folding or aggregation or abnormal amyloid formation, deposition, accumulation or persistence, or amyloid lipid interactions. In aspects of the invention, the term refers to conditions associated with the formation, deposition, accumulation, or persistence of amyloid or amyloid fibrils, comprising an amyloid protein selected from the group consisting of Aβ amyloid, AA amyloid, AL amyloid, IAPP amyloid, PrP amyloid, α₂-microglobulin amyloid, transthyretin, prealbumin, and procalcitonin, especially Aβ amyloid and IAPP amyloid. A “disease” may be a condition where it is desirable to dissociate abnormally aggregated proteins and/or dissolve or disrupt pre-formed or pre-deposited amyloid or amyloid fibril.

In certain aspects of the invention the disease is amyloidosis. “Amyloidosis” refers to a diverse group of diseases of acquired or hereditary origin and characterized by the accumulation of one of several different types of protein fibrils with similar properties called amyloid. Amyloid can accumulate in a single organ or be dispersed throughout the body. The disease can cause serious problems in the affected areas, which may include the heart, brain, kidneys and digestive tract. The fibrillar composition of amyloid deposits is an identifying characteristic for various amyloid diseases. Intracerebral and cerebrovascular deposits composed primarily of fibrils of beta amyloid peptide (β-AP) are characteristic of Alzheimer's disease (both familial and sporadic forms), islet amyloid protein peptide (IAPP; amylin) is characteristic of the fibrils in pancreatic islet cell amyloid deposits associated with type II diabetes, and β-2-microglobulin is a major component of amyloid deposits which form as a consequence of long term hemodialysis treatment. Prion-associated diseases, such as Creutzfeld-Jacob disease, scrapie, bovine spongiform encephalitis, and the like are characterized by the accumulation of a protease-resistant form of a prion protein (designated as AScr ro PrP-27).

Certain disorders are considered to be primary amyloidoses, in which there is no evidence for preexisting or coexisting disease. Primary amyloidoses are typically characterized by the presence of “amyloid light chain-type” (AL-type) protein fibrils. In secondary amyloidosis there is an underlying chronic inflammatory or infectious disease state (e.g., rheumatoid arthritis, juvenile chronic arthritis, ankylosing spondylitis, psoriasis, Reiter's syndrome, Adult Still's disease, Behcet's Syndrome, Crohn's disease, chronic microbial infections such as osteomyelitis, tuberculosis, and leprosy, malignant neoplasms such as Hodgkin's lymphoma, renal carcinoma, carcinomas of the gut, lung, and urogenital tract, basel cell carcinoma, and hairy cell carcinoma). Secondary amyloidosis is characterized by deposition of AA type fibrils derived from serum amyloid A protein (ApoSSA). Heredofamilial amyloidoses may have associated neuropathic, renal, or cardiovascular deposits of the ATTR transthyretin type, and they include other syndromes having different amyloid components (e.g., familial Mediterranean fever which is characterized by AA fibrils). Other forms of amyloidosis include local forms, characterized by focal, often tumor-like deposits that occur in isolated organs. In addition, amyloidoses are associated with aging, and are commonly characterized by plaque formation in the heart or brain. Amyloidoses includes systemic diseases such as adult-onset diabetes, complications from long-term hemodialysis and consequences of chronic inflammation or plasma cell dyscrasias.

In aspects of the invention, amyloid diseases that can be treated and/or prevented using the compounds, compositions and methods of the invention include without limitation, Alzheimer's disease, Down's syndrome, dementia pugilistica, multiple system atrophy, inclusion body myositosis, hereditary cerebral hemorrhage with amyloidosis of the Dutch type, Nieman-Pick disease type C, cerebral β-amyloid angiopathy, dementia associated with cortical basal degeneration, the amyloidosis of type II diabetes, the amyloidosis of chronic inflammation, the amyloidosis of malignancy and Familial Mediterranean Fever, the amyloidosis of multiple myeloma and B-cell dyscrasias, nephropathy with urticaria and deafness (Muckle-Wells syndrome), amyloidosis associated with systemic inflammatory diseases, idiopathic primary amyloidosis associated with myeloma or macroglobulinemia; amyloidosis associated with immunocyte dyscrasia; monoclonal gammopathy; occult dyscrasia; local nodular amyloidosis associated with chronic inflammatory diseases; amyloidosis associated with several immunocyte dyscrasias; familial amyloid polyneuropathy; hereditary cerebral hemorrhage with amyloidosis Alzheimer's disease and other neurodegenerative diseases, amyloidosis associated with chronic hemodialysis and insulinoma, the amyloidosis of the prion diseases, (transmissible spongiform encephalopathies prion diseases), Creutzfeldt-Jakob disease, Gerstmann-Straussler syndrome, Kuru, scrapie, the amyloidosis associated with carpal tunnel syndrome, senile cardiac amyloidosis, familial amyloidotic polyneuropathy, and the amyloidosis associated with endocrine tumors, especially Alzheimer's disease and type 2 diabetes.

In aspects of the invention, diseases that can be treated and/or prevented using the compounds, compositions and methods of the invention include conditions of the central or peripheral nervous system or a systemic organ that result in the deposition of proteins, protein fragments, and peptides in beta-pleated sheets, fibrils, and/or aggregates or oligomers. In particular the disease is Alzheimer's disease, presenile and senile forms; amyloid angiopathy; mild cognitive impairment; Alzheimer's disease-related dementia (e.g., vascular or Alzheimer dementia); tauopathy (e.g., argyrophilic grain dementia, corticobasal degeneration, dementia pugilistica, diffuse neurofibrillary tangles with calcification, frontotemporal dementia with parkinsonism, Prion-related disease, Hallervorden-Spatz disease, myotonic dystrophy, Niemann-Pick disease type C, non-Guamanian Motor Neuron disease with neurofibrillary tangles, Pick's disease, postencephalitic parkinsonism, cerebral amyloid angiopathy, progressive subcortical gliosis, progressive supranuclear palsy, subacute sclerosing panencephalitis, and tangle only dementia), alpha-synucleinopathy (e.g., dementia with Lewy bodies, multiple system atrophy with glial cytoplasmic inclusions, Shy-Drager syndrome, spinocerebellar ataxia (e.g., DRPLA or Machado-Joseph Disease); striatonigral degeneration, olivopontocerebellar atrophy, neurodegeneration with brain iron accumulation type I, olfactory dysfunction, and amyotrophic lateral sclerosis); Parkinson's disease (e.g., familial or non-familial); Amyotrophic Lateral Sclerosis; Spastic paraplegia (e.g., associated with defective function of chaperones and/or triple A proteins); Huntington's Disease, spinocerebellar ataxia, Freidrich's Ataxia; neurodegenerative diseases associated with intracellular and/or intraneuronal aggregates of proteins with polyglutamine, polyalanine or other repeats arising from pathological expansions of tri- or tetra-nucleotide elements within corresponding genes; cerebrovascular diseases; Down's syndrome; head trauma with post-traumatic accumulation of amyloid beta peptide; Prion related disease (Creutzfeldt-Jakob disease, Gerstmann-Straussler-Scheinker disease, and variant Creutzfeldt-Jakob disease); Familial British Dementia; Familial Danish Dementia; Presenile Dementia with Spastic Ataxia; Cerebral Amyloid Angiopathy, British Type; Presenile Dementia With Spastic Ataxia Cerebral Amyloid Angiopathy, Danish Type; Familial encephalopathy with neuroserpin inclusion bodies (FENIB); Amyloid Polyneuropathy (e.g., senile amyloid polyneuropathy or systemic Amyloidosis); Inclusion Body myositis due to amyloid beta peptide; Familial and Finnish Type Amyloidosis; Systemic amyloidosis associated with multiple myeloma; Familial Mediterranean Fever; chronic infections and inflammations; and type II diabetes mellitus associated with islet amyloid polypeptide (IAPP).

In selected aspects of the invention, the disease is a neuronal disorder (e.g., Alzheimer's disease, Down Syndrome, Parkinson's disease, Chorea Huntington, pathogenic psychotic conditions, schizophrenia, impaired food intake, sleep-wakefulness, impaired homeostatic regulation of energy metabolism, impaired autonomic function, impaired hormonal balance, impaired regulation, body fluids, hypertension, fever, sleep dysregulation, anorexia, anxiety related disorders including depression, seizures including epilepsy, drug withdrawal and alcoholism, disorders including cognitive dysfunction and dementia).

In certain selected aspects of the invention, the disease is a neurodegenerative disease or neurodegenerative disorder including such diseases and impairments as Alzheimer's disease, dementia, MCI, Huntington's disease, Parkinson's disease, amyotrophic lateral sclerosis, epilepsy, Pick's disease, and other similar diseases and disorders disclosed herein.

The compounds of the invention may also act to inhibit or prevent α-synuclein/NAC fibril formation, inhibit or prevent α-synuclein/NAC fibril growth, and/or cause disassembly, disruption, and/or disaggregation of preformed α-synuclein/NAC fibrils and α-synuclein/NAC-associated protein deposits. Examples of synuclein diseases or synucleinopathies suitable for treatment with a compound or composition of the invention are diseases associated with the formation, deposition, accumulation, or persistence of synuclein fibrils, especially α-synuclein fibrils, including without limitation Parkinson's disease, familial Parkinson's disease, Lewy body disease, the Lewy body variant of Alzheimer's disease, dementia with Lewy bodies, multiple system atrophy, olivopontocerebellar atrophy, neurodegeneration with brain iron accumulation type I, olfactory dysfunction, and the Parkinsonism-dementia complex of Guam.

In an aspect of the invention, the disease is a Motor Neuron Disease associated with filaments and aggregates of neurofilaments and/or superoxide dismutase proteins, the Spastic paraplegia associated with defective function of chaperones and/or triple A proteins and the spinocerebellar ataxia is DRPLA or Machado-Joseph Disease.

In other aspects, the disease is a Prion Disease including Creutzfeldt-Jakob disease, Gerstmann-Strausller-Scheinfer disease, and variant Creutzfeldt-Jakob disease and an Amyloid Polyneuropathy including senile amyloid polyneuropathy or systemic amyloidosis.

In an embodiment, the disease is Alzheimer's disease or Parkinson's disease including familial and non-familial types. In particular embodiments of the invention, the disease is Alzheimer's disease.

In certain aspects of the invention, the disease may be characterized by an inflammatory process due to the presence of macrophages by, an amyloidogenic protein or peptide. A method of the invention may involve inhibiting macrophage activation and/or inhibiting an inflammatory process. A method may comprise decreasing, slowing, ameliorating, or reversing the course or degree of macrophage invasion or inflammation in a patient.

A disease may be a condition that is associated with a molecular interaction that can be disrupted or dissociated with a compound of the invention. “A molecular interaction that can be disrupted or dissociated with a compound of the invention” includes an interaction comprising an amyloid protein and a protein or glycoprotein. An interaction comprising an amyloid protein includes an amyloid protein-amyloid protein interaction, amyloid-proteoglycan interaction, amyloid-proteoglycan/glycosaminoglycan (GAG) interaction and/or amyloid protein-glycosaminoglycan interaction. An interacting protein may be a cell surface, secreted or extracellular protein.

A disease that may be treated or prevented using a compound or composition of the invention includes a disease that would benefit from the disruption or dissolution of a molecular interaction comprising an amyloid protein and an interacting compound including a protein or glycoprotein. Examples of diseases that may be treated or prevented using a compound or composition of the invention include infectious diseases caused by bacteria, viruses, prions and fungi. Examples of such disorders and/or diseases are those associated with pathogens including Herpes simplex virus, Pseudorabies virus, human cytomegalovirus, human immunodeficiency virus, Bordetella pertussis, Chlamydia trachomatis, Haemophilus influenzae, Helicobacter pylori, Borrelia burgdorferi, Neisseria gonorrhoeae, Mycobacterium tuberculosis, Staphylococcus aureus, Streptococcus mutans, Streptococcus suis, Plasmodium falciparum, Leishmania amazonensi, Trypanozoma cruzi, Listeria monocytogenes, Mycoplasma pneumoniae, enterotoxigenic E. coli, uropathogenic E. coli, and Pseudomonas aeruginosa.

The term “interaction” or “interacting” refers to any physical, association between proteins, other molecules such as lipids, carbohydrates, nucleotides, and other cell metabolites. Examples of interactions include protein-protein interactions. The term preferably refers to a stable association between two molecules due to, for example, electrostatic, hydrophobic, ionic and/or hydrogen-bond interactions under physiological conditions. Certain interacting or associated molecules interact only after one or more of them has been stimulated (e.g. phosphorylated). An interaction between proteins and other cellular molecules may be either direct or indirect.

Compounds

The invention provides an isolated, in particular pure, more particularly substantially pure, compound of the formula I, wherein X is a radical of scyllo-inositol or a configuration isomer thereof, wherein one or more of, two or more of, or three or more of R¹, R², R³, R⁴, R⁵, and R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide and the other of R¹, R², R³, R⁴, R⁵, or R⁶ is a hydroxyl with the proviso that when (a) one of R¹, R², R³, R⁴, R⁵, and R⁶ are alkyl or fluorine no more than 4 of the other of R¹, R², R³, R⁴, R⁵, and R⁶ are hydroxyl, (b) one of R¹, R², R³, R⁴, R⁵, and R⁶ is amino or azide no more than four of R¹, R², R³, R⁴, R⁵, and R⁶ are hydroxyl, (c) two of R¹, R², R³, R⁴, R⁵, and R⁶ are amino, no more than three of R¹, R², R³, R⁴, R⁵, and R⁶ are hydroxyl, and (d) R¹, R², R³, R⁴, R⁵, and R⁶ cannot be isopropylidene.

In an aspect the invention provides an isolated, in particular pure, more particularly, substantially pure, compound of the formula II wherein one or more of, two or more of, or three or more of R¹, R², R³, R⁴, R⁵, and R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfinyl, sulfonate, amino, imino, azido, thiol, thioalkyl, thioalkoxy, thioaryl, nitro, cyano, isocyanato, halo, seleno, silyl, silyloxy, silylthio, carboxyl, carbonyl, or carbamoyl, carboxamide and the other of R¹, R², R³, R⁴, R⁵, or R⁶ is a hydroxyl with the proviso that when (a) one of R¹, R², R³, R⁴, R⁵, and R⁶ are alkyl or fluorine no more than 4 of the other of R¹, R², R³, R⁴, R⁵, and R⁶ are hydroxyl, (b) one of R¹, R², R³, R⁴, R⁵, and R⁶ is amino or azide no more than four of R¹, R², R³, R⁴, R⁵, and R⁶ are hydroxyl, (c) two of R¹, R², R³, R⁴, R⁵, and R⁶ are amino, no more than three of R¹, R², R³, R⁴, R⁵, and R⁶ are hydroxyl, and (d) R¹, R², R³, R⁴, R⁵, and R⁶ cannot be isopropylidene.

In an aspect of the invention, a compound of the formula I or II is provided wherein one or more of, two or more of, or three or more of R¹, R², R³, R⁴, R⁵, and R⁶ are independently alkenyl, alkynyl, alkylene, alkenylene, alkoxy, alkenyloxy, cycloalkenyl, cycloalkoxy, aryl, aryloxy, arylalkoxy, aroyl, heteroaryl, heterocyclic, acyl, acyloxy, sulfonyl, sulfenyl, sulfinyl, sulfonate, sulfoxide, sulfate, nitro, cyano, isocyanato, thioaryl, thioalkoxy, seleno, silyl, silyloxy, silylthio, Cl, I, Br, carboxyl, carbonyl, carbamoyl, or carboxamide and the other of R¹, R², R³, R⁴, R⁵, or R⁶ is a hydroxyl.

In another aspect of the invention a compound of the formula I is provided wherein R² is hydroxyl in an equatorial position, at least one, two, three, or four of R¹, R³, R⁴, R⁵, and R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfenyl, sulfonyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide, and the other of R¹, R³, R⁴, R⁵, and R⁶ are hydroxyl.

In another aspect of the invention a compound of the formula I is provided wherein R² is hydroxyl in an equatorial position, at least two of R¹, R³, R⁴, R⁵, and R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide, and the other of R¹, R³, R⁴, R⁵, and R⁶ are hydroxyl.

In a particular aspect, a compound of the formula I is provided wherein R² is hydroxyl in an equatorial position, at least one, two, three, or four of R¹, R², R³, R⁴, R⁵, and R⁶ are independently alkenyl, alkynyl, alkylene, alkenylene, alkoxy, alkenyloxy, cycloalkyl, cycloalkenyl, aryl, aryloxy, arylalkoxy, aroyl, heteroaryl, heterocyclic, acyl, nitro, cyano, nitro, cyano, isocyanato, Cl, Br, I, acyloxy, sulfonyl, sulfenyl, sulfinyl, sulfonate, sulfoxide, sulfate, thioalkoxy, thioaryl, carboxyl, seleno, silyl, silyloxy, silylthio, carbonyl, carbamoyl, or carboxamide, and the other of R¹, R³, R⁴, R⁵, and R⁶ are hydroxyl.

In a further aspect the invention provides a compound of the formula I or II wherein two of R¹, R², R³, R⁴, R⁵, and R⁶ are hydroxyl, and two or more of the other of R¹, R², R³, R⁴, R⁵, and R⁶ are alkyl, cycloalkyl, alkenyl, cycloalkenyl, alkynyl, alkylene, alkenylene, alkoxy, alkenyloxy, cycloalkoxy, aryl, aryloxy, arylalkoxy, aroyl, heteroaryl, heterocyclic, acyl, or acyloxy, sulfonyl, sulfenyl, sulfinyl, amino, imino, cyano, isocyanato, seleno, silyl, silyloxy, silylthio, thiol, thioalkyl, thioalkoxy, halo, carboxyl, carbonyl, carbamoyl, and carboxamide.

In a further aspect the invention provides a compound of the formula I or II wherein two of R¹, R², R³, R⁴, R⁵, and R⁶ are hydroxyl, and three or more of the other of R¹, R², R³, R⁴, R⁵, and R⁶ are alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, azido, nitro, cyano, isocyanato, halo, seleno, silyl, silyloxy, silylthio, carboxyl, carbonyl, carbamoyl, or carboxamide.

In a further aspect the invention provides a compound of the formula I or II wherein two of R¹, R², R³, R⁴, R⁵, and R⁶ are hydroxyl, and one, two or four of the other of R¹, R², R³, R⁴, R⁵, and R⁶ are alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide.

In a still further aspect the invention provides a compound of the formula I or II wherein three of R¹, R², R³, R⁴, R⁵, and R⁶ are hydroxyl, and one, two, or three of the other of R¹, R², R³, R⁴, R⁵, and R⁶ are alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide.

In a still further aspect the invention provides a compound of the formula I or II wherein three of R¹, R², R³, R⁴, R⁵, and R⁶ are hydroxyl, and one, two or three of the other of R¹, R², R³, R⁴, R⁵, and R⁶ are alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide.

In a still further aspect the invention provides a compound of the formula I or II wherein four of R¹, R², R³, R⁴, R⁵, and R⁶ are hydroxyl, and one or two of the other of R¹, R³, R⁴, R⁵, and R⁶ is alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfonate, sulfenyl, sulfinyl, amino, imino, azido, thiol, thioalkyl, thioalkoxy, thioaryl, azido, nitro, cyano, isocyanato, halo, seleno, silyl, silyloxy, silylthio, carboxyl, carbonyl, carbamoyl, or carboxamide.

In a particular aspect of the invention a compound of the formula I is provided wherein R¹, R², R⁴, R⁵, and R⁶ are hydroxyl, and R³ is alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, azido, nitro, cyano, isocyanato, halo, seleno, silyl, silyloxy, silylthio, carboxyl, carbonyl, carbamoyl, or carboxamide and the other of R¹, R³, R⁴, R⁵, and R⁶ is hydroxyl. In an embodiment, R³ is selected from the group consisting of alkenyl, alkynyl, alkylene, alkenylene, alkoxy, alkenyloxy, cycloalkyl, cycloalkenyl, cycloalkoxy, aryl, aryloxy, arylalkoxy, aroyl, imino, heteroaryl, heterocyclic, acyl, acyloxy, sulfonyl, sulfenyl, sulfinyl, sulfoxide, sulfate, thioalkoxy, thioaryl, carboxyl, carbonyl, carbamoyl, or carboxamide, in particular alkoxy, sulfonyl, sulfenyl, sulfinyl, sulfoxide, sulfate, thioalkoxy, carboxyl, carbonyl, carbamoyl, or carboxamide.

In embodiments of the invention, two, three, four or five of R¹, R², R³, R⁴, R⁵, or R⁶ are hydroxyl, the other of R¹, R², R³, R⁴, R⁵, or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide, especially alkyl, amino, imino, azido, thiol, thioalkyl, nitro, thioalkoxy, cyano, or halo, and at least one of R¹, R², R³, R⁴, R⁵, or R⁶ is alkoxy, in particular alkoxy having about 1-6 carbon atoms, more particularly methoxy, ethoxy, propoxy, butoxy, isopropoxy and tert-butoxy, substituted with halo, alkyl, substituted alkyl, or cycloalkyl, in particular substituted with alkyl, halo (e.g., fluoro), alkylhalo, haloalkylhalo, alkylhaloalkyl, cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl).

In embodiments of the invention, two of R¹, R², R³, R⁴, R⁵, or R⁶ are hydroxyl, the other of R¹, R², R³, R⁴, R⁵, or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide, especially alkyl, amino, imino, azido, thiol, thioalkyl, nitro, thioalkoxy, cyano, or halo, and at least one of R¹, R², R³, R⁴, R⁵, or R⁶ is alkoxy, in particular alkoxy having about 1-6 carbon atoms, more particularly methoxy, ethoxy, propoxy, butoxy, isopropoxy and tert-butoxy, substituted with halo, alkyl, or substituted alkyl, in particular substituted with alkyl, halo (e.g., fluoro), alkylhalo, haloalkylhalo, alkylhaloalkyl, cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl).

In embodiments of the invention, three of R¹, R², R³, R⁴, R⁵, or R⁶ are hydroxyl, the other of R¹, R², R³, R⁴, R⁵, or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide, especially alkyl, amino, imino, azido, thiol, thioalkyl, nitro, thioalkoxy, cyano, or halo, and at least one of R¹, R², R³, R⁴, R⁵, or R⁶ is alkoxy, in particular alkoxy having about 1-6 carbon atoms, more particularly methoxy, ethoxy, propoxy, butoxy, isopropoxy and tert-butoxy, substituted with halo, alkyl, or substituted alkyl, in particular substituted with alkyl, halo (e.g., fluoro), alkylhalo, haloalkylhalo, alkylhaloalkyl, cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl).

In embodiments of the invention, four of R¹, R², R³, R⁴, R⁵, or R⁶ are hydroxyl, the other of R¹, R², R³, R⁴, R⁵, or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide, especially alkyl, amino, imino, azido, thiol, thioalkyl, nitro, thioalkoxy, cyano, or halo, and at least one of R¹, R², R³, R⁴, R⁵, or R⁶ is alkoxy, in particular alkoxy having about 1-6 carbon atoms, more particularly methoxy, ethoxy, propoxy, butoxy, isopropoxy and tert-butoxy, substituted with halo, alkyl, or substituted alkyl, in particular substituted with alkyl, halo (e.g., fluoro), alkylhalo, haloalkylhalo, alkylhaloalkyl, cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl)l.

In embodiments of the invention, five of R¹, R², R³, R⁴, R⁵, or R⁶ are hydroxyl and the other of R¹, R², R³, R⁴, R⁵, or R⁶ is alkoxy, in particular alkoxy having about 1-6 carbon atoms, more particularly methoxy, ethoxy, propoxy, butoxy, isopropoxy and tert-butoxy, substituted with halo, alkyl, or substituted alkyl, in particular substituted with alkyl, halo (e.g., fluoro), alkylhalo, haloalkylhalo, alkylhaloalkyl, cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl).

In selected compounds of the above embodiments of the invention, at least one of R¹, R², R³, R⁴, R⁵, or R⁶ is —OR²⁰ wherein R²⁰ is —CF₃, CF₃CF₂, CF₃CH₂, CH₂NO₂, CH₂NH₂, C(CH₂)₃, or cyclopropyl.

In embodiments of the invention, R¹, R², R³, R⁴, and R⁵ are hydroxyl and R⁶ is alkoxy, in particular alkoxy having about 1-6 carbon atoms, more particularly methoxy, ethoxy, propoxy, butoxy, isopropoxy and tert-butoxy, substituted with halo, alkyl, or substituted alkyl, in particular substituted with alkyl, halo (e.g., fluoro), alkylhalo, haloalkylhalo, alkylhaloalkyl, cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In a particular embodiment of the invention, R¹, R², R³, R⁴, and R⁵ are hydroxyl and R⁶ is —OR²⁰ wherein R²⁰ is CF₃, CF₃CF₂, CF₃CH₂, CH₂NO₂, CH₂NH₂, C(CH₂)₃, or cyclopropyl.

In embodiments of the invention, R¹, R², R³, R⁴, and R⁶ are hydroxyl and R⁵ is alkoxy, in particular alkoxy having about 1-6 carbon atoms, more particularly methoxy, ethoxy, propoxy, butoxy, isopropoxy and tert-butoxy, substituted with halo, alkyl, or substituted alkyl, in particular substituted with alkyl, halo (e.g., fluoro), alkylhalo, haloalkylhalo, alkylhaloalkyl, cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In a particular embodiment of the invention, R¹, R², R³, R⁴, and R⁶ are hydroxyl and R⁵ is —OR²⁰ wherein R²⁰ is CF₃, CF₃CF₂, CF₃CH₂, CH₂NO₂, CH₂NH₂, C(CH₂)₃, or cyclopropyl.

In embodiments of the invention, R¹, R², R³, R⁵, and R⁶ are hydroxyl and R⁴ is a is alkoxy, in particular alkoxy having about 1-6 carbon atoms, more particularly methoxy, ethoxy, propoxy, butoxy, isopropoxy and tert-butoxy, substituted with halo, alkyl, or substituted alkyl, in particular substituted with alkyl, halo (e.g., fluoro), alkylhalo, haloalkylhalo, alkylhaloalkyl, cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In particular embodiments of the invention, R¹, R², R³, R⁵, and R⁶ are hydroxyl and R⁴ is —OR²⁰ wherein R²⁰ is CF₃, CF₃CF₂, CF₃CH₂, CH₂NO₂, CH₂NH₂, C(CH₂)₃, or cyclopropyl.

In embodiments of the invention, R¹, R², R⁴, R⁵, and R⁶ are hydroxyl and R³ is alkoxy, in particular alkoxy having about 1-6 carbon atoms, more particularly methoxy, ethoxy, propoxy, butoxy, isopropoxy and tert-butoxy, substituted with halo, alkyl, or substituted alkyl, in particular substituted with alkyl, halo (e.g., fluoro), alkylhalo, haloalkylhalo, alkylhaloalkyl, cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In particular embodiments of the invention, R¹, R², R⁴, R⁵, and R⁶ are hydroxyl and R³ is —OR²⁰ wherein R²⁰ is CF₃, CF₃CF₂, CF₃CH₂, CH₂NO₂, CH₂NH₂, C(CH₂)₃, or cyclopropyl.

In embodiments of the invention, R¹, R³, R⁴, R⁵, and R⁶ are hydroxyl and R² is alkoxy, in particular alkoxy having about 1-6 carbon atoms, more particularly methoxy, ethoxy, propoxy, butoxy, isopropoxy and tert-butoxy, substituted with halo, alkyl, or substituted alkyl, in particular substituted with alkyl, halo (e.g., fluoro), alkylhalo, haloalkylhalo, alkylhaloalkyl, cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In particular embodiments of the invention, R¹, R³, R⁴, R⁵, and R⁶ are hydroxyl and R² is —OR²⁰ wherein R²⁰ is CF₃, CF₃CF₂, CF₃CH₂, CH₂NO₂, CH₂NH₂, C(CH₂)₃, or cyclopropyl.

In embodiments of the invention, R², R³, R⁴, R⁵, and R⁶ are hydroxyl and R¹ is alkoxy, in particular alkoxy having about 1-6 carbon atoms, more particularly methoxy, ethoxy, propoxy, butoxy, isopropoxy and tert-butoxy, substituted with halo, alkyl, or substituted alkyl, in particular substituted with alkyl, halo (e.g., fluoro), alkylhalo, haloalkylhalo, alkylhaloalkyl, cyano, amino, nitro, or cycloalkyl, more particularly CF₃, CF₃CF₂, CF₃CH₂, CH₂NO₂, CH₂NH₂, C(CH₂)₃, or a 3-4 membered cycloalkyl (e.g. cyclopropyl). In particular embodiments of the invention, R², R³, R⁴, R⁵, and R⁶ are hydroxyl and R¹ is —OR²⁰ wherein R²⁰ is CF₃, CF₃CF₂, CF₃CH₂, CH₂NO₂, CH₂NH₂, C(CH₂)₃, or cyclopropyl.

In embodiments of the invention, two, three, four or five of R¹, R², R³, R⁴, R⁵, or R⁶ are hydroxyl, the other of R¹, R², R³, R⁴, R⁵, or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide, especially alkyl, amino, imino, azido, thiol, thioalkyl, nitro, thioalkoxy, cyano, or halo, and at least one of R¹, R², R³, R⁴, R⁵, or R⁶ is a carboxylic ester. In aspects of the invention at least one of R¹, R², R³, R⁴, R⁵, or R⁶ is —C(O)OR²¹ where R²¹ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, two of R¹, R², R³, R⁴, R⁵, or R⁶ are hydroxyl, the other of R¹, R², R³, R⁴, R⁵, or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide, especially alkyl, amino, imino, azido, thiol, thioalkyl, nitro, thioalkoxy, cyano, or halo, and at least one of R¹, R², R³, R⁴, R⁵, or R⁶ is a carboxylic ester.

In embodiments of the invention, three of R¹, R², R³, R⁴, R⁵, or R⁶ are hydroxyl, the other of R¹, R², R³, R⁴, R⁵, or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide, especially alkyl, amino, imino, azido, thiol, thioalkyl, nitro, thioalkoxy, cyano, or halo, and at least one of R¹, R², R³, R⁴, R⁵, or R⁶ is a carboxylic ester.

In embodiments of the invention, four of R¹, R², R³, R⁴, R⁵, or R⁶ are hydroxyl, the other of R¹, R², R³, R⁴, R⁵, or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide, especially alkyl, amino, imino, azido, thiol, thioalkyl, nitro, thioalkoxy, cyano, or halo, and at least one of R¹, R², R³, R⁴, R⁵, or R⁶ is a carboxylic ester.

In embodiments of the invention, five of R¹, R², R³, R⁴, R⁵, or R⁶ are hydroxyl and the other of R¹, R¹, R¹, R¹, R⁵, or R⁶ is a carboxylic ester.

In selected aspects of these embodiments of the invention at least one of R¹, R², R³, R⁴, R⁵, or R⁶ is —C(O)OR²¹ where R²¹ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, R¹, R², R³, R⁴, and R⁵ are hydroxyl and R⁶ is a carboxylic ester. In aspects of the invention, R⁶ is —C(O)OR²¹ where R²¹ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, R¹, R², R³, R⁴, and R⁶ are hydroxyl and R⁵ is a carboxylic ester. In aspects of the invention, R⁵ is —C(O)OR²¹ where R²¹ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, R¹, R², R³, R⁵, and R⁶ are hydroxyl and R⁴ is a carboxylic ester. In aspects of the invention, R⁴ is —C(O)OR²¹ where R²¹ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, R¹, R², R⁴, R⁵, and R⁶ are hydroxyl and R³ is a carboxylic ester. In aspects of the invention, R³ is —C(O)OR²¹ where R²¹ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, R¹, R³, R⁴, R⁵, and R⁶ are hydroxyl and R² is a carboxylic ester. In aspects of the invention, R² is —C(O)OR²¹ where R²¹ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In embodiments of the invention, R², R³, R⁴, R⁵, and R⁶ are hydroxyl and R¹ is a carboxylic ester. In aspects of the invention, R¹ is —C(O)OR²¹ where R²¹ is hydrogen, alkyl, alkenyl, alkynyl, cycloalkyl, cycloalkenyl, amino, thiol, aryl, heteroaryl, thioalkyl, thioaryl, thioalkoxy, or a heterocyclic ring, which may optionally be substituted, in particular substituted with alkyl substituted with one or more of alkyl, amino, halo, alkylamino, aryl, carboxyl, aryl, or a heterocyclic.

In particular embodiments, R²¹ is selected to provide an amino acid derivative or an ester derivative. In preferred embodiments of the invention R²¹ is one of the following:

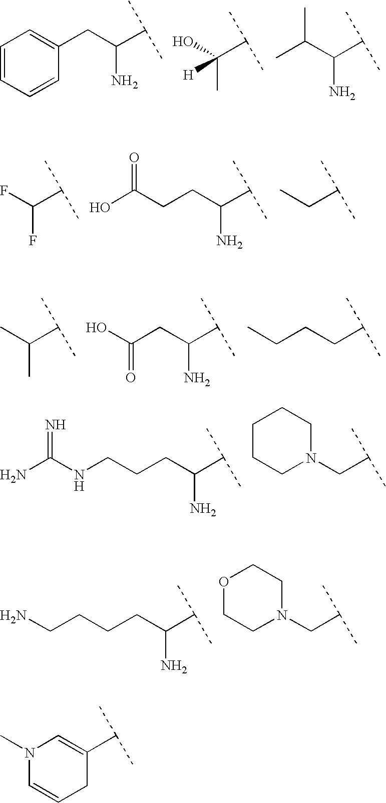

In aspects, the invention provides compounds of the formula I or II wherein one, two, three, four or five of R¹, R², R³, R⁴, R⁵, or R⁶ are each independently:
(a) alkyl with 1 to 24 carbon atoms, in particular 1 to 10 or 1 to 6 carbon atoms;
(b) cycloalkyl with 3 to 16 carbon atoms, in particular 3 to 10 or 3 to 6 carbon atoms;
(c) alkenyl with 2 to 24 carbon atoms, in particular 2 to 10 or 2 to 6 carbon atoms;
(d) cycloalkenyl with 4 to 16 carbon atoms, in particular 4 to 10 or 4 to 6 carbon atoms;
(e) aryl with 4 to 24 carbon atoms, in particular 4 to 10, 4 to 8, or 6 or carbon atoms;
(f) aralkyl, alkaryl, aralkenyl, or alkenylaryl;
(g) heterocyclic group comprising at least one atom selected from the group consisting of oxygen, nitrogen, and sulfur;
(h) alkoxy with 1 to 6 carbon atoms in particular methoxy, ethoxy, propoxy, butoxy, isopropoxy or tert-butoxy, especially methoxy; or
(i) halo, in particular fluorine, chlorine, or bromine, especially chlorine.

In an aspect, the invention provides a compound of the formula I or II wherein R² is hydroxyl and one, two, three, four or five of R¹, R³, R⁴, R⁵, or R⁶ is each independently methyl, ethyl, propyl, butyl, pentyl, hexyl, heptyl, octyl, nonyl, decyl, dodecyl, tetradecyl, pentadecyl, hexadecyl, heptadecyl, octadecyl, eicosyl, docosyl, cyclopropyl, cyclopentyl, cyclohexyl, vinyl, allyl, propenyl, octadienyl, octenyl, decenyl, dodecenyl, tetradecenyl, hexadecenyl, octadecenyl, octadecadienyl, nonadecenyl, octadecatrienyl, arachidonyl, cyclopentenyl, cycopentadienyl, cyclohexenyl, cyclohexadienyl, phenyl, biphenyl, terphenyl, naphtyl, anthracenyl, phenanthrenyl, pyridyl, furyl, or thiazolyl.

In a particular aspect, the invention provides a compound of the formula I or II wherein one, two, or three of R¹, R², R³, R⁴, R⁵, or R⁶ is each independently —OR²⁵ where R²⁵ is alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide or a carbohydrate.

In a particular aspect, the invention provides a compound of the formula I or II wherein one, two or three of R¹, R², R³, R⁴, R⁵, or R⁶ is each independently 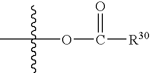
where R³⁰ is alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide, and the other of R¹, R², R³, R⁴, R⁵, or R⁶ is hydroxyl.

The invention provides a compound of the formula I or II wherein at least one, two, three or four of R¹, R³, R⁴, R⁵, and R⁶ are hydroxyl and the other of R¹, R³, R⁴, R⁵, and R⁶ are alkyl, halo, alkoxy, sulfonyl, sulfinyl, thiol, thioalkyl, thioalkoxy, carboxyl.

The invention further provides a compound of the formula I or II wherein R¹, R², R³, R⁴, R⁵, or R⁶ is each independently F, N₃, NH₂, SH, NO₂, CF₃, OCF₃, SeH, Cl, Br, I or CN with the proviso that four or five of R¹, R², R³, R⁴, R⁵, or R⁶ are hydroxyl.

In particular aspects of the invention, five of R¹, R², R³, R⁴, R⁵, or R⁶ are hydroxyl and one of R¹, R², R³, R⁴, R⁵, or R⁶, and more particularly R³, is selected from the group consisting of F, SeH, Cl, Br, I and CN.

In other particular aspects of the invention, four of R¹, R², R³, R⁴, R⁵, or R⁶ are hydroxyl and two of R¹, R², R³, R⁴, R⁵, or R⁶ are selected from the group consisting of F, —NO₂, SH, SeH, Cl, Br, I and CN.

In further particular aspects of the invention, four of R¹, R², R³, R⁴, R⁵, or R⁶ are hydroxyl and the other two of R¹, R², R³, R⁴, R⁵, or R⁶ are lower alkyl, especially methyl, ethyl, butyl, or propyl, preferably methyl.

In further particular aspects of the invention, four of R¹, R², R³, R⁴, R⁵, or R⁶ are hydroxyl and the other two of R¹, R², R³, R⁴, R⁵, or R⁶ are lower cycloalkyl, especially cyclopropyl, cyclobutyl, and cyclopentyl.

In a still further particular aspect of the invention, one or two of R¹, R², R³, R⁴, R⁵, or R⁶ are carboxyl, carbamyl, sulfonyl, or a heterocyclic comprising a N atom, more particularly N-methylcarbamyl, N-propylcarbamyl, N-cyanocarbamyl, aminosulfonyl, isoxazolyl, imidazolyl, and thiazolyl.

In embodiments of the invention, two, three, four or five of R¹, R², R³, R⁴, R⁵, or R⁶ are hydroxyl, the other of R¹, R², R³, R⁴, R⁵, or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide, especially alkyl, amino, imino, azido, thiol, thioalkyl, nitro, thioalkoxy, cyano, or halo, and at least one of R¹, R², R³, R⁴, R⁵, or R⁶ is alkoxy, in particular alkoxy having about 1-6 carbon atoms, more particularly methoxy, ethoxy, propoxy, butoxy, isopropoxy or tert-butoxy.

In embodiments of the invention, two of R¹, R², R³, R⁴, R⁵, or R⁶ are hydroxyl, the other of R¹, R², R³, R⁴, R⁵, or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide, especially alkyl, amino, imino, azido, thiol, thioalkyl, nitro, thioalkoxy, cyano, or halo, and at least one of R¹, R², R³, R⁴, R⁵, or R⁶ is alkoxy, in particular alkoxy having about 1-6 carbon atoms, more particularly methoxy, ethoxy, propoxy, butoxy, isopropoxy or tert-butoxy.

In embodiments of the invention, three of R¹, R², R³, R⁴, R⁵, or R⁶ are hydroxyl, the other of R¹, R², R³, R⁴, R⁵, or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide, especially alkyl, amino, imino, azido, thiol, thioalkyl, nitro, thioalkoxy, cyano, or halo, and at least one of R¹, R², R³, R⁴, R⁵, or R⁶ is alkoxy, in particular alkoxy having about 1-6 carbon atoms, more particularly methoxy, ethoxy, propoxy, butoxy, isopropoxy or tert-butoxy.

In embodiments of the invention, four of R¹, R², R³, R⁴, R⁵, or R⁶ are hydroxyl, the other of R¹, R², R³, R⁴, R⁵, or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide, especially alkyl, amino, imino, azido, thiol, thioalkyl, nitro, thioalkoxy, cyano, or halo, and at least one of R¹, R², R³, R⁴, R⁵, or R⁶ is alkoxy, in particular alkoxy having about 1-6 carbon atoms, more particularly methoxy, ethoxy, propoxy, butoxy, isopropoxy and tert-butoxy.

In embodiments of the invention, five of R¹, R², R³, R⁴, R⁵, or R⁶ are hydroxyl and the other of R¹, R², R³, R⁴, R⁵, or R⁶ is alkoxy, in particular alkoxy having about 1-6 carbon atoms, more particularly methoxy, ethoxy, propoxy, butoxy, isopropoxy and tert-butoxy, especially methoxy.

In embodiments of the invention, R¹, R², R³, R⁴, and R⁵ are hydroxyl and R⁶ is alkoxy, in particular alkoxy having about 1-6 carbon atoms, more particularly methoxy, ethoxy, propoxy, butoxy, isopropoxy and tert-butoxy. In a particular embodiment of the invention, R¹, R², R³, R⁴, and R⁵ are hydroxyl and R⁶ is methoxy.

In embodiments of the invention, R¹, R², R³, R⁴, and R⁶ are hydroxyl and R⁵ is alkoxy, in particular alkoxy having about 1-6 carbon atoms, more particularly methoxy, ethoxy, propoxy, butoxy, isopropoxy and tert-butoxy. In a particular embodiment of the invention, R¹, R², R³, R⁴, and R⁶ are hydroxyl and R⁵ is methoxy.

In embodiments of the invention, R¹, R², R³, R⁵, and R⁶ are hydroxyl and R⁴ is alkoxy, in particular alkoxy having about 1-6 carbon atoms, more particularly methoxy, ethoxy, propoxy, butoxy, isopropoxy and tert-butoxy. In a particular embodiment of the invention, R¹, R², R³, R⁵, and R⁶ are hydroxyl and R⁴ is methoxy.

In embodiments of the invention, R¹, R², R⁴, R⁵, and R⁶ are hydroxyl and R³ is alkoxy, in particular alkoxy having about 1-6 carbon atoms, more particularly methoxy, ethoxy, propoxy, butoxy, isopropoxy and tert-butoxy. In a particular embodiment of the invention, R¹, R², R⁴, R⁵, and R⁶ are hydroxyl and R³ is methoxy.

In embodiments of the invention, R¹, R³, R⁴, R⁵, and R⁶ are hydroxyl and R² is alkoxy, in particular alkoxy having about 1-6 carbon atoms, more particularly methoxy, ethoxy, propoxy, butoxy, isopropoxy and tert-butoxy. In a particular embodiment of the invention, R¹, R³, R⁴, R⁵, and R⁶ are hydroxyl and R² is methoxy.

In embodiments of the invention, R², R³, R⁴, R⁵, and R⁶ are hydroxyl and R¹ is alkoxy, in particular alkoxy having about 1-6 carbon atoms, more particularly methoxy, ethoxy, propoxy, butoxy, isopropoxy and tert-butoxy. In a particular embodiment of the invention, R², R³, R⁴, R⁵, and R⁶ are hydroxyl and R¹ is methoxy.

In selected embodiments of the invention, the compound is methyl-scyllo-inositol, more particularly compound ID 260 in Table 1.

In embodiments of the invention, five of R¹, R², R³, R⁴, R⁵, or R⁶ are hydroxyl and the other of R¹, R², R³, R⁴, R⁵, or R⁶ is substituted alkoxy, in particular alkoxy having about 1-6 carbon atoms, more particularly methoxy, ethoxy, propoxy, butoxy, isopropoxy and tert-butoxy substituted with alkyl, in particular lower alkyl.

In embodiments of the invention, five of R¹, R², R³, R⁴, R⁵, or R⁶ are hydroxyl and the other of R¹, R², R³, R⁴, R⁵, or R⁶ is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments five of R¹, R², R³, R⁴, R⁵, or R⁶ are hydroxyl and the other of R¹, R², R³, R⁴, R⁵, or R⁶ is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, five of R¹, R², R³, R⁴, R⁵, or R⁶ are hydroxyl and the other of R¹, R², R³, R⁴, R⁵, or R⁶ is a haloalkoxyalkyl, in particular fluoromethoxymethyl, chloromethoxyethyl, trifluoromethoxymethyl, difluoromethoxyethyl, or trifluoroethoxymethyl.

In embodiments of the invention, R¹, R², R³, R⁴, and R⁵ are hydroxyl and R⁶ is substituted alkoxy, in particular alkoxy having about 1-6 carbon atoms, more particularly methoxy, ethoxy, propoxy, butoxy, isopropoxy and tert-butoxy substituted with alkyl, in particular lower alkyl.

In embodiments of the invention, R¹, R², R³, R⁴, and R⁶ are hydroxyl and R⁵ is substituted alkoxy, in particular alkoxy having about 1-6 carbon atoms, more particularly methoxy, ethoxy, propoxy, butoxy, isopropoxy and tert-butoxy substituted with alkyl, in particular lower alkyl.

In embodiments of the invention, R¹, R², R³, R⁵, and R⁶ are hydroxyl and R⁴ is substituted alkoxy, in particular alkoxy having about 1-6 carbon atoms, more particularly methoxy, ethoxy, propoxy, butoxy, isopropoxy and tert-butoxy substituted with alkyl, in particular lower alkyl.

In embodiments of the invention, R¹, R², R⁴, R⁵, and R⁶ are hydroxyl and R³ is substituted alkoxy, in particular alkoxy having about 1-6 carbon atoms, more particularly methoxy, ethoxy, propoxy, butoxy, isopropoxy and tert-butoxy substituted with alkyl, in particular lower alkyl.

In embodiments of the invention, R¹, R³, R⁴, R⁵, and R⁶ are hydroxyl and R² is substituted alkoxy, in particular alkoxy having about 1-6 carbon atoms, more particularly methoxy, ethoxy, propoxy, butoxy, isopropoxy and tert-butoxy substituted with alkyl, in particular lower alkyl.

In embodiments of the invention, R², R³, R⁴, R⁵, and R⁶ are hydroxyl and R¹ is substituted alkoxy, in particular alkoxy having about 1-6 carbon atoms, more particularly methoxy, ethoxy, propoxy, butoxy, isopropoxy and tert-butoxy substituted with alkyl, in particular lower alkyl.

In embodiments of the invention, R¹, R², R³, R⁴, and R⁵ are hydroxyl and R⁶ is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R¹, R², R³, R⁴, and R⁵ are hydroxyl and R⁶ is is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, R¹, R², R³, R⁴, and R⁶ are hydroxyl and R⁵ is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R¹, R², R³, R⁴, and R⁶ are hydroxyl and R⁵ is is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, R¹, R², R³, R⁵, and R⁶ are hydroxyl and R⁴ is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R¹, R², R³, R⁴, and R⁶ are hydroxyl and R⁵ is is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, R¹, R², R⁴, R⁵, and R⁶ are hydroxyl and R³ is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R¹, R², R⁴, R⁵, and R⁶ are hydroxyl and R³ is is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, R¹, R³, R⁴, R⁵, and R⁶ are hydroxyl and R² is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R¹, R³, R⁴, R⁵, and R⁶ are hydroxyl and R² is is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, R², R³, R⁴, R⁵, and R⁶ are hydroxyl and R¹ is alkoxy, in particular alkoxy having about 1-6 carbon atoms, more particularly methoxy, ethoxy, propoxy, butoxy, isopropoxy and tert-butoxy, substituted with halo (e.g., fluoro, chloro or bromo). In particular embodiments R², R³, R⁴, R⁵, and R⁶ are hydroxyl and R¹ is is fluoromethoxy, chloromethoxy, trifluoromethoxy, difluoromethoxy, trifluoroethoxy, fluoroethoxy, tetrafluoroethoxy, pentafluoroethoxy, or fluoropropoxy.

In embodiments of the invention, two, three, four or five of R¹, R², R³, R⁴, R⁵, or R⁶ are hydroxyl, the other of R¹, R², R³, R⁴, R⁵, or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide, especially alkyl, amino, imino, azido, thiol, thioalkyl, nitro, thioalkoxy, cyano, or halo and at least one of R¹, R², R³, R⁴, R⁵, or R⁶ is halo, in particular fluoro, chloro or bromo, more particularly chloro.

In embodiments of the invention, two of R¹, R², R³, R⁴, R⁵, or R⁶ are hydroxyl, the other of R¹, R², R³, R⁴, R⁵, or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide, especially alkyl, amino, imino, azido, thiol, thioalkyl, nitro, thioalkoxy, cyano, or halo, and at least one of R¹, R², R³, R⁴, R⁵, or R⁶ is halo, in particular fluoro, chloro or bromo, more particularly chloro.

In embodiments of the invention, three of R¹, R², R³, R⁴, R⁵, or R⁶ are hydroxyl, the other of R¹, R², R³, R⁴, R⁵, or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide, especially alkyl, amino, imino, azido, thiol, thioalkyl, nitro, thioalkoxy, cyano, or halo, and at least one of R¹, R², R³, R⁴, R⁵, or R⁶ is halo, in particular fluoro, chloro or bromo, more particularly chloro.

In embodiments of the invention, four of R¹, R², R³, R⁴, R⁵, or R⁶ are hydroxyl, the other of R¹, R², R³, R⁴, R⁵, or R⁶ are independently alkyl, alkenyl, alkynyl, alkylene, alkenylene, alkoxy, alkenyloxy, cycloalkyl, cycloalkenyl, cycloalkoxy, aryl, aryloxy, arylalkoxy, aroyl, heteroaryl, heterocyclic, acyl, acyloxy, sulfoxide, sulfate, sulfonyl, sulfenyl, sulfonate, sulfinyl, amino, imino, azido, thiol, thioalkyl, thioalkoxy, thioaryl, nitro, cyano, isocyanato, halo, seleno, silyl, silyloxy, silylthio, carboxyl, carbonyl, carbamoyl, or carboxamide, especially alkyl, amino, imino, azido, thiol, thioalkyl, nitro, thioalkoxy, cyano, or halo, and at least one of R¹, R², R³, R⁴, R⁵, or R⁶ is halo, in particular fluoro, chloro or bromo, more particularly chloro.

In embodiments of the invention, five of R¹, R², R³, R⁴, R⁵, or R⁶ are hydroxyl and the other of R¹, R², R³, R⁴, R⁵, or R⁶ is halo, in particular fluoro, chloro or bromo, more particularly chloro.

In embodiments of the invention, R¹, R², R³, R⁴, and R⁵ are hydroxyl and R⁶ is halo, in particular fluorine, chlorine or bromine, more particularly chloro. In a particular embodiment of the invention, R¹, R², R³, R⁴, and R⁵ are hydroxyl and R⁶ is chloro.

In embodiments of the invention, R¹, R², R³, R⁴, and R⁶ are hydroxyl and R⁵ is halo, in particular fluoro, chloro or bromo, more particularly chloro. In a particular embodiment of the invention, R¹, R², R³, R⁴, and R⁶ are hydroxyl and R⁵ is chloro.

In embodiments of the invention, R¹, R², R³, R⁵, and R⁶ are hydroxyl and R⁴ is halo, in particular fluoro, chloro or bromo, more particularly chloro. In a particular embodiment of the invention, R¹, R², R³, R⁵, and R⁶ are hydroxyl and R⁴ is chloro.

In embodiments of the invention, R¹, R², R⁴, R⁵, and R⁶ are hydroxyl and R³ is halo, in particular fluoro, chloro or bromo, more particularly chloro. In a particular embodiment of the invention, R¹, R², R⁴, R⁵, and R⁶ are hydroxyl and R³ is chloro.

In embodiments of the invention, R¹, R³, R⁴, R⁵, and R⁶ are hydroxyl and R² is halo, in particular fluoro, chloro or bromo, more particularly chloro. In a particular embodiment of the invention, R¹, R³, R⁴, R⁵, and R⁶ are hydroxyl and R² is chloro.

In embodiments of the invention, R², R³, R⁴, R⁵, and R⁶ are hydroxyl and R¹ is halo, in particular fluoro, chloro or bromo, more particularly chloro. In a particular embodiment of the invention, R², R³, R⁴, R⁵, and R⁶ are hydroxyl and R¹ is chloro.

In selected embodiments of the invention, the compound is 1-chloro-1-deoxy-scyllo-inositol, as structurally depicted in Table 1.

A compound of the invention may additionally comprise a carrier, including with out limitation one or more of a polymer, carbohydrate, peptide or derivative thereof. A carrier may be substituted with substituents described herein including without limitation one or more alkyl, amino, nitro, halogen, thiol, thioalkyl, sulfate, sulfonyl, sulfenyl, sulfinyl, sulfoxide, hydroxyl groups. A carrier can be directly or indirectly covalently attached to a compound of the invention. In aspects of the invention the carrier is an amino acid including alanine, glycine, praline, methionine, serine, threonine, or asparagine. In other aspects the carrier is a peptide including alanyl-alanyl, prolyl-methionyl, or glycyl-glycyl.

A carrier also includes a molecule that targets a compound of the invention to a particular tissue or organ. In particular, a carrier may facilitate or enhance transport of a compound of the invention to the brain by either active or passive transport.

In an embodiment, the invention provides a compound of the formula I or II wherein at least one of R¹, R³, R⁴, R⁵, and R⁶ is a sulfonate group which is optionally attached directly or indirectly to a carrier, in particular a carbohydrate. The number of sulfonate groups may be selected to provide a beneficial effect.

Process

The compounds of the formula I or II of this invention may be prepared using reactions and methods generally known to the person of ordinary skill in the art, having regard to that knowledge and the disclosure of this application including the Examples. The reactions are performed in a solvent appropriate to the reagents and materials used and suitable for the reactions being effected. It will be understood by those skilled in the art of organic synthesis that the functionality present on the compounds should be consistent with the proposed reaction steps. This will sometimes require modification of the order of the synthetic steps or selection of one particular process scheme over another in order to obtain a desired compound of the invention. It will also be recognized that another major consideration in the development of a synthetic route is the selection of the protecting group used for protection of the reactive functional groups present in the compounds described in this invention. An authoritative account describing the many alternatives to the skilled artisan is Greene and Wuts (Protective Groups In Organic Synthesis, Wiley and Sons, 1991).

The starting materials and reagents used in preparing compounds or the invention are either available from commercial suppliers such as the Aldrich Chemical Company (Milwaukee, Wis.), Bachem (Torrance, Calif.), Sigma (St. Louis, Mo.), or Lancaster Synthesis Inc. (Windham, N.H.) or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989. Publications disclosing particular processes for preparing scyllo-inositol include Husson, C., et al, Carbohyrate Research 307 (1998) 163-165) and Sarmah, M. P. and Shashidar, M. S., Carbohydrate Research 338 (2003) 999-1001.

The starting materials, intermediates, and compounds of this invention may be isolated and purified using conventional techniques, such as precipitation, filtration, distillation, crystallization, chromatography, and the like. The compounds may be characterized using conventional methods, including physical constants and spectroscopic methods, in particular HPLC.

The compounds of the formula I or II which are basic in nature can form a wide variety of different salts with various inorganic and organic acids. In practice is it desirable to first isolate a compound of the formula I from the reaction mixture as a pharmaceutically unacceptable salt and then convert the latter to the free base compound by treatment with an alkaline reagent and subsequently convert the free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent such as methanol or ethanol. Upon careful evaporation of the solvent, the desired solid salt is obtained.

Compounds of the formula I or II which are acidic in nature are capable of forming base salts with various pharmacologically acceptable cations. These salts may be prepared by conventional techniques by treating the corresponding acidic compounds with an aqueous solution containing the desired pharmacologically acceptable cations and then evaporating the resulting solution to dryness, preferably under reduced pressure. Alternatively, they may be prepared by mixing lower alkanolic solutions of the acidic compounds and the desired alkali metal alkoxide together and then evaporating the resulting solution to dryness in the same manner as before. In either case, stoichiometric quantities of reagents are typically employed to ensure completeness of reaction and maximum product yields.

Compositions and Kits

A compound of the formula I or II of the invention may be formulated into a pharmaceutical composition or dietary supplement for administration to a subject. Pharmaceutical compositions of the present invention or fractions thereof comprise suitable pharmaceutically acceptable carriers, excipients, and vehicles selected based on the intended form of administration, and consistent with conventional pharmaceutical practices.

Suitable pharmaceutical carriers, excipients, and vehicles are described in the standard text, Remington's Pharmaceutical Sciences, Mack Publishing Company. By way of example for oral administration in the form of a capsule or tablet, the active components can be combined with an oral, non-toxic pharmaceutically acceptable inert carrier such as lactose, starch, sucrose, methyl cellulose, magnesium stearate, glucose, calcium sulfate, dicalcium phosphate, mannitol, sorbital, and the like. For oral administration in a liquid form, the drug components may be combined with any oral, non-toxic, pharmaceutically acceptable inert carrier such as ethanol, glycerol, water, and the like. Suitable binders (e.g. gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, and coloring agents may also be combined in the compositions or components thereof. Compositions as described herein can further comprise wetting or emulsifying agents, or pH buffering agents.

A composition of the invention can be a liquid solution, suspension, emulsion, tablet, pill, capsule, sustained release formulation, or powder. The compositions can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, etc. Various delivery systems are known and can be used to administer a composition of the invention, e.g. encapsulation in liposomes, microparticles, microcapsules, and the like.

Formulations for parenteral administration may include aqueous solutions, syrups, aqueous or oil suspensions and emulsions with edible oil such as cottonseed oil, coconut oil or peanut oil. Dispersing or suspending agents that can be used for aqueous suspensions include synthetic or natural gums, such as tragacanth, alginate, acacia, dextran, sodium carboxymethylcellulose, gelatin, methylcellulose, and polyvinylpyrrolidone.

Compositions for parenteral administration may include sterile aqueous or non-aqueous solvents, such as water, isotonic saline, isotonic glucose solution, buffer solution, or other solvents conveniently used for parenteral administration of therapeutically active agents. A composition intended for parenteral administration may also include conventional additives such as stabilizers, buffers, or preservatives, e.g. antioxidants such as methylhydroxybenzoate or similar additives.

Compositions of the invention can be formulated as pharmaceutically acceptable salts as described herein.

A composition of the invention may be sterilized by, for example, filtration through a bacteria retaining filter, addition of sterilizing agents to the composition, irradiation of the composition, or heating the composition. Alternatively, the compounds or compositions of the present invention may be provided as sterile solid preparations e.g. lyophilized powder, which are readily dissolved in sterile solvent immediately prior to use.

After pharmaceutical compositions have been prepared, they can be placed in an appropriate container and labeled for treatment of an indicated condition. For administration of a composition of the invention, such labeling would include amount, frequency, and method of administration.

A compound of the formula I or II may be in a form suitable for administration as a dietary supplement. A supplement of the invention may optionally include inactive ingredients such as diluents or fillers, viscosity-modifying agents, preservatives, flavorings, colorants, or other additives conventional in the art. By way of example only, conventional ingredients such as beeswax, lecithin, gelatin, glycerin, caramel, and carmine may be included.

A dietary supplement composition of the invention may optionally comprise a second active ingredient. In an embodiment, the second active ingredient is pinitol or an active derivative or metabolite thereof. Pinitol can be produced from plant sources, including without limitation alfalfa, Bougainvillea leaves, chick peas, pine trees and soy beans. Pinitol is also commercially available, for example Inzitol™ (Humanetics Corporation, Min). Examples of derivatives and metabolites of pinitol include without limitation pinitol glycosides, pinitol phospholipids, esterified pinitol, lipid-bound pinitol, pinitol phosphates, pinitol phytates, and hydrolyzed pinitol such as d-chiro-inositol.

A dietary supplement may be provided as a liquid dietary supplement e.g., a dispensable liquid) or alternatively the compositions may be formulated as granules, capsules or suppositories. The liquid supplement may include a number of suitable carriers and additives including water, glycols, oils, alcohols, flavoring agents, preservatives, coloring agents and the like. In capsule, granule or suppository form, the compositions of the present invention are formulated in admixture with a pharmaceutically acceptable carrier.

A supplement may be presented in the form of a softgel which is prepared using conventional methods. A softgel typically includes a layer of gelatin encapsulating a small quantity of the supplement. A supplement may also be in the form of a liquid-filled and sealed gelatin capsule, which may be made using conventional methods.

To prepare a dietary supplement composition of the present invention in capsule, granule or suppository form, one or more compositions of the present invention may be intimately admixed with a pharmaceutically acceptable carrier according to conventional formulation techniques. For solid oral preparations such as capsules and granules, suitable carriers and additives such as starches, sugars, diluents, granulating agents, lubricants, binders, disintegrating agents and the like may be included.

In embodiments of the invention, a pharmaceutical pack or kit is provided comprising one or more containers filled with one or more of the ingredients of a pharmaceutical composition of the invention to provide a beneficial effect, in particular a sustained beneficial effect. Associated with such container(s) can be various written materials such as instructions for use, or a notice in the form prescribed by a governmental agency regulating the labeling, manufacture, use or sale of pharmaceuticals or biological products, which notice reflects approval by the agency of manufacture, use, or sale for human administration.

According to another aspect of the invention, a kit is provided. In an aspect, the kit comprises a compound or a pharmaceutical composition of the invention. The kit can be a package which houses a container which contains a composition of the invention and also houses instructions for administering the composition to a subject.

Applications

The invention contemplates the use of a composition of the invention for treating a disease, in particular preventing, and/or ameliorating disease severity, disease symptoms, and/or periodicity of recurrence of a disease disclosed herein. The invention also contemplates treating in mammals diseases using the compositions or treatments of the invention. The present invention in embodiments may provide a composition comprising a compound that provides beneficial effects including greater solubility, stability, efficacy, potency, and/or utility, in particular greater solubility and stability.

In an aspect of the invention a compound of the formula I or II is utilized in the treatment of Alzheimer's disease. Thus, Alzheimer's disease may be treated by administering a therapeutically effective amount of a compound of the formula I or formula II. Such treatment may be effective for retarding the degenerative effects of Alzheimer's disease, including specifically, but not exclusively, deterioration of the central nervous system, loss of mental facilities, loss of short term memory, and disorientation.

In an embodiment, where the disease is Alzheimer's disease, beneficial effects of a compound or composition or treatment of the invention can manifest as one, two, three, four, five, six, seven, eight, nine, or all of the following, in particular five or more, more particularly 8 or more of the following:
a) An increase or restoration of long term potentiation relative to the level in the absence of a compound disclosed herein after administration to a subject with symptoms of Alzheimer's disease. In aspects of the invention a compound disclosed herein induces at least about a 0.05%, 0.1%, 0.5%, 1%, 2%, 5%, 10%, 15%, 20%, 30%, 33%, 35%, 40%, 45%, 50%, 60%, 70%, 80%, 90%, 95%, or 99% increase in long term potentiation in a subject.
b) An increase or maintenance of synaptic function relative to the level of synaptic function in the absence of a compound disclosed herein after administration to a subject with symptoms of Alzheimer's disease. In aspects of the invention a compound disclosed herein induces at least about a 0.05%, 0.1%, 0.5%, 1%, 2%, 5%, 10%, 15%, 20%, 30%, 33%, 35%, 40%, 45%, 50%, 60%, 70%, 80%, 90%, 95%, 99%, 100%, 125%, 150%, 175% or 200% increase in synaptic function in a subject.
c) An increase in synaptophysin. In aspects of the invention there is at least about a 2%, 5%, 10%, 15%, 20%, 30%, 40%, 50%, 60%, 70%, 80%, 90%, 95%, 99%, 100%, 125%, 150%, 175% or 200% increase in synaptophysin.
d) An increase in synaptophysin reactive boutons and cell bodies. In aspects of the invention there is at least about a 2%, 5%, 10%, 15%, 20%, 30%, 40%, 50%, 60%, 70%, 80%, 90%, 95%, 99%, 100%, 125%, 150%, 175% or 200%, more particularly about a 100-150% or 140-150% increase in synaptophysin reactive boutons and cell bodies.
e) A reduction, slowing or prevention of an increase in, or an absence of symptoms of inflammation, in particular an Aβ-induced inflammatory response, after administration to a subject with symptoms of Alzheimer's disease.
f) A reduction, slowing or prevention of an increase in cerebral accumulation of amyloid β relative to the levels measured in the absence of a compound disclosed herein in subjects with symptoms of Alzheimer's disease. In aspects of the invention, the compound induces at least about a 2%, 5%, 10%, 15%, 20%, 30%, 40%, 50%, 60%, 70%, 80%, or 90% decrease in cerebral accumulation of amyloid β.
g) A reduction, slowing or prevention of an increase in deposition of cerebral amyloid plaques, relative to the levels measured in the absence of a compound disclosed herein in subjects with symptoms of Alzheimer's disease. In aspects of the invention, the compound induces at least about a 2%, 5%, 10%, 15%, 20%, 30%, 40%, 50%, 60%, 70%, 80%, or 90% decrease in deposition of cerebral amyloid plaques.
h) A reduction, slowing or prevention of an increase in plaque number. In aspects of the invention, a compound disclosed herein induces at least about a 2%, 5%, 10%, 15%, 20%, 30%, 40%, 50%, 60%, 70%, 80%, or 90% reduction in plaque number. In particular aspects the compound induces a 5-15% or 10-15% reduction in plaque number.
i) A reduction, slowing or prevention of an increase in plaque size. In aspects of the invention, a compound disclosed herein induces at least about a 2%, 5%, 10%, 15%, 20%, 30%, 40%, 50%, 60%, 70%, 80%, or 90% reduction in plaque size. In particular aspects the compound induces a 5-15% or 10-15% reduction in plaque size.
j) A reduction, slowing or prevention of an increase in percent area of the brain covered in plaques. In aspects of the invention, a compound disclosed herein induces at least about a 2%, 5%, 10%, 15%, 20%, 30%, 40%, 50%, 60%, 70%, 80%, or 90% reduction in percent area of the brain covered in plaques. In particular aspects the compound induces a 5-15% or 10-15% reduction in percent area of the brain covered in plaques.
k) A reduction, slowing or prevention of an increase in soluble Aβ oligomers in the brain, relative to the levels measured in the absence of a compound disclosed herein in subjects with symptoms of Alzheimer's disease. In aspects of the invention, the combination induces at least about a 2%, 5%, 10%, 15%, 20%, 30%, 40%, 50%, 60%, 70%, 80%, or 90% decrease in soluble Aβ oligomers.
l) A reduction, slowing or prevention of an increase in brain levels of Aβ40. In aspects of the invention, a compound disclosed herein induces at least about a 2%, 5%, 10%, 15%, 20%, 30%, 40%, 50%, 60%, 70%, 80%, or 90% reduction in Aβ40. In particular aspects the compound induces a 10-50%, 20-45%, or 25-35% reduction in brain levels of Aβ40.
m) A reduction, slowing or prevention of an increase in Aβ42 levels in a body fluid such as CSF or blood. In aspects of the invention, a compound disclosed herein induces at least about a 2%, 5%, 10%, 15%, 20%, 30%, 40%, 50%, 60%, 70%, 80%, or 90% reduction in A42. In particular aspects the compound induces a 10-50%, 15-40%, or 20-25% reduction in brain levels of Aβ42.
n) A reduction, slowing or prevention of an increase in brain levels of Aβ42. In aspects of the invention, a compound disclosed herein induces at least about a 2%, 5%, 10%, 15%, 20%, 30%, 40%, 50%, 60%, 70%, 80%, or 90% reduction in Aβ42. In particular aspects the compound induces a 10-50%, 15-40%, or 20-25% reduction in brain levels of Aβ42.
o) A reduction, slowing or prevention of an increase in glial activity in the brain, relative to the levels measured in the absence of a compound disclosed herein in subjects with symptoms of Alzheimer's disease. Preferably, the compound induces at least about a 2%, 5%, 10%, 15%, 20%, 30%, 40%, 50%, 60%, 70%, 80%, or 90% decrease in glial activity
p) Maintenance of synaptic function at about normal for a prolonged period of time, in particular for at least 5 weeks, 6 weeks, 8 weeks, 10 weeks, 12 weeks, 14 weeks, 16 weeks, 20 weeks, 24 weeks, 30 weeks, 40 weeks, 52 weeks, or 78 weeks, more particularly, 2 to 4 weeks, 2 to 5 weeks, 3 to 5 weeks, 2 to 6 weeks, 2 to 8 weeks, 2 to 10 weeks, 2 to 12 weeks, 2 to 16 weeks, 2 to 20 weeks, 2 to 24 weeks, 2 weeks to 12 months, or 2 weeks to 24 months following treatment.
q) A reduction or slowing of the rate of disease progression in a subject with Alzheimer's disease. In particular a reduction or slowing of cognitive decline in a subject with Alzheimer's disease.
r) A reduction, slowing or prevention of an increase in cognitive deficits.
s) A reduction, slowing or prevention of an increase in amyloid angiopathy.
t) A reduction in accelerated mortality.
u) An increase in survival in a subject with symptoms of Alzheimer's disease.

In aspects of the invention beneficial effects of a composition or treatment of the invention can manifest as (a) and (b); (a), (b) and (c); (a), (b), (e), (f) and (g); (a), (b), (e), (f) through (h); (a), (b), (e), (f) through (i); (a), (b), (e), (f) through (j); (a), (b), (e), (f) through (k); (a), (b), (e), (f) through (l); (a), (b), (e), (f) through (m); (a), (b), (e), (f) through (n); (a), (b), (e), (f) through (O); (a), (b), (e), (f) through (p); (a), (b), (e), (f) through (q); (a), (b), (e), (f) through (r); (a), (b), (e), (f) through (s); (a), (b), (e), (f) through (t); (a) through (d); (a) through (e); (a) through (f); (a) through (g); (a) through (h); (a) through (i); (a) through (j); (a) through (k); (a) through (l); (a) through (m); (a) through (n); (a) through (O); (a) through (p); (a) through (q); (a) through (r); (a) through (s); (a) through (t), or (a) through (u).

Compounds, pharmaceutical compositions and methods of the invention can be selected that have sustained beneficial effects, preferably statistically significant sustained beneficial effects. In an embodiment, a pharmaceutical composition with statistically significant sustained beneficial effects is provided comprising a therapeutically effective amount of a compound of the invention.

Greater efficacy and potency of a treatment of the invention in some aspects may improve the therapeutic ratio of treatment, reducing untoward side effects and toxicity. Selected methods of the invention may also improve long-standing Alzheimer's disease even when treatment is begun long after the appearance of symptoms. Prolonged efficacious treatment can be achieved in accordance with the invention following administration of a compound or composition of the invention.

In an aspect, the invention relates to a method for treating Alzheimer's disease comprising contacting Aβ or Aβ aggregates, in particular Aβ40 or Aβ40 aggregates and/or Aβ42 or Aβ42 aggregates, in a subject with a therapeutically effective amount of a compound or a composition of the invention.

In another aspect, the invention provides a method for treating Alzheimer's disease by providing a composition comprising a compound of the invention in an amount sufficient to disrupt aggregated Aβ for a prolonged period following administration.

In a further aspect, the invention provides a method for treating Alzheimer's disease in a patient in need thereof which includes administering to the individual a composition that provides a compound of the invention in a dose sufficient to increase inhibition of long term potentiation induced by Aβ oligomers and/or maintain synaptic function. In another aspect, the invention provides a method for treating Alzheimer's disease comprising administering, preferably orally or systemically, an amount of a compound of the invention to a mammal, to reduce cerebral accumulation of AD, deposition of cerebral amyloid plaques, soluble Aβ oligomers in the brain, glial activity, and/or inflammation for a prolonged period following administration.

The invention in an embodiment provides a method for treating Alzheimer's disease, the method comprising administering to a mammal in need thereof a composition comprising a compound of the invention in an amount sufficient to reduce cognitive decline for a prolonged period following administration, thereby treating the Alzheimer's disease.

In another aspect, the invention provides a method for preventing and/or treating Alzheimer's disease, the method comprising administering to a mammal in need thereof a composition comprising a compound of the invention in an amount sufficient to disrupt aggregated Aβ for a prolonged period following administration; and determining the amount of aggregated Aβ, thereby treating the Alzheimer's disease. The amount of aggregated Aβ may be measured using an antibody specific for AD or a compound of the invention labeled with a detectable substance.

The present invention also includes methods of using the compositions of the invention in combination with one or more additional therapeutic agents including without limitation beta-secretase inhibitors, alpha-secretase inhibitors, and epsilon-secretase inhibitors, agents that are used for the treatment of complications resulting from or associated with a disease, or general medications that treat or prevent side effects.

The invention also contemplates the use of a composition comprising at least one compound of the invention for the preparation of a medicament in treating a disorder or disease.

In an embodiment, the invention relates to the use of a therapeutically effective amount of at least one compound of the invention for preparation of a medicament for providing therapeutic effects, in particular beneficial effects, preferably sustained beneficial effects, in treating a disorder or disease.

In a still further embodiment the invention provides the use of a compound of the invention for the preparation of a medicament for prolonged or sustained treatment of Alzheimer's disease.

Therapeutic efficacy and toxicity of compositions and methods of the invention may be determined by standard pharmaceutical procedures in cell cultures or with experimental animals such as by calculating a statistical parameter such as the ED₅₀ (the dose that is therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions which exhibit large therapeutic indices are preferred. By way of example, one or more of the therapeutic effects, in particular beneficial effects disclosed herein, can be demonstrated in a subject or disease model, for example, a TgCRND8 mouse with symptoms of Alzheimer's disease.

Administration

Compounds and compositions of the present invention can be administered by any means that produce contact of the active agent(s) with the agent's sites of action in the body of a subject or patient to produce a therapeutic effect, in particular a beneficial effect, in particular a sustained beneficial effect. The active ingredients can be administered simultaneously or sequentially and in any order at different points in time to provide the desired beneficial effects. A compound and composition of the invention can be formulated for sustained release, for delivery locally or systemically. It lies within the capability of a skilled physician or veterinarian to select a form and route of administration that optimizes the effects of the compositions and treatments of the present invention to provide therapeutic effects, in particular beneficial effects, more particularly sustained beneficial effects.

The compositions may be administered in oral dosage forms such as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular forms, all utilizing dosage forms well known to those of ordinary skill in the pharmaceutical arts. The compositions of the invention may be administered by intranasal route via topical use of suitable intranasal vehicles, or via a transdermal route, for example using conventional transdermal skin patches. A dosage protocol for administration using a transdermal delivery system may be continuous rather than intermittent throughout the dosage regimen. A sustained release formulation can also be used for the therapeutic agents.

The dosage regimen of the invention will vary depending upon known factors such as the pharmacodynamic characteristics of the agents and their mode and route of administration; the species, age, sex, health, medical condition, and weight of the patient, the nature and extent of the symptoms, the kind of concurrent treatment, the frequency of treatment, the route of administration, the renal and hepatic function of the patient, and the desired effect.

An amount of a therapeutic of the invention which will be effective in the treatment of a particular disorder or disease to provide effects, in particular beneficial effects, more particularly sustained beneficial effects, will depend on the nature of the condition or disorder, and can be determined by standard clinical techniques. The precise dose to be employed in the formulation will also depend on the route of administration, and the seriousness of the disease, and should be decided according to the judgement of the practitioner and each patient's circumstances.

Suitable dosage ranges for administration are particularly selected to provide therapeutic effects, in particular beneficial effects, more particularly sustained beneficial effects. A dosage range is generally effective for triggering the desired biological responses. The dosage ranges are generally about 0.1 mg to about 2 kg per kg per day, about 0.5 mg to about 2 g per kg per day, about 1 mg to about 1 g per kg per day, about 1 mg to about 200 mg per kg per day, about 1 mg to about 100 mg per kg per day, about 10 mg to about 100 mg per kg, 30 mg to 70 mg per kg per day, about 1 mg to about 50 mg per kg per day, about 2 to about 50 mg/kg/day, about 2 mg to about 40 mg per kg, or about 3 mg to 30 mg per kg per day. In aspects of the invention, the dosage ranges are generally about 0.5 mg to about 2 g per kg, about 1 mg to about 1 g per kg, about 1 mg to about 200 mg per kg, about 1 mg to about 100 mg per kg, about 1 mg to about 50 mg per kg, about 10 mg to about 100 mg per kg, or about 30 mg to 70 mg per kg of the weight of a subject.

In some aspects of the invention, the dosage ranges of a compound disclosed herein administered once twice, three times or more daily, especially once or twice daily, are about 1 to 100 mg/kg, 1 to 90 mg/kg, 1 to 80 mg/kg, 1 to 75 mg/kg, 1 to 70 mg/kg, 1 to 60 mg/kg, 1 to 50 mg/kg, 1 to 40 mg/kg, 1 to 35 mg/kg, 2 to 35 mg/kg, 2.5 to 30 mg/kg, 3 to 30 mg/kg, 3 to 20 mg/kg, or 3 to 15 mg/kg. In embodiments of the invention, the required dose of a compound disclosed herein administered twice daily is about 1 to 50 mg/kg, 1 to 40 mg/kg, 2.5 to 40 mg/kg, 3 to 40 mg/kg, 3 to 35 mg/kg, most preferably 3 to 30 mg/kg. In embodiments of the invention, the required daily dose of the compound is about 1 to 80 mg/kg and within that range 1 to 70 mg/kg, 1 to 65 mg/kg, 2 to 70 mg/kg, 3 to 70 mg/kg, 4 to 65 mg/kg, 5 to 65 mg/kg, or 6 to 60 mg/kg.

In embodiments of the invention, the required dose of a compound disclosed herein, administered twice daily is about 1 to 50 mg/kg, 1 to 40 mg/kg, 2.5 to 40 mg/kg, 3 to 40 mg/kg, 3 to 35 mg/kg, most preferably 3 to 30 mg/kg.

In other embodiments of the invention, the required daily dose of a compound disclosed herein, is about 1 to 80 mg/kg and within that range 1 to 70 mg/kg, 1 to 65 mg/kg, 2 to 70 mg/kg, 3 to 70 mg/kg, 4 to 65 mg/kg, 5 to 65 mg/kg, or 6 to 60 mg/kg.

A composition or treatment of the invention may comprise a unit dosage of at least one compound of the invention to provide beneficial effects. A “unit dosage” or “dosage unit” refers to a unitary i.e. a single dose which is capable of being administered to a patient, and which may be readily handled and packed, remaining as a physically and chemically stable unit dose comprising either the active agents as such or a mixture with one or more solid or liquid pharmaceutical excipients, carriers, or vehicles.

A subject may be treated with a compound of the formula I or II or composition or formulation thereof on substantially any desired schedule. A composition of the invention may be administered one or more times per day, in particular 1 or 2 times per day, once per week, once a month or continuously. However, a subject may be treated less frequently, such as every other day or once a week, or more frequently. A compound, composition or formulation of the invention may be administered to a subject for about or at least about 1 week, 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, 2 weeks to 18 months, or 2 weeks to 24 months, periodically or continuously.

In an aspect, the invention provides a regimen for supplementing a human's diet, comprising administering to the human a supplement comprising a compound of the formula I or II, or nutraceutically acceptable derivatives thereof. A subject may be treated with a supplement at least about every day, or less frequently, such as every other day or once a week. A supplement of the invention may be taken daily but consumption at lower frequency, such as several times per week or even isolated doses, may be beneficial.

In a particular aspect, the invention provides a regimen for supplementing a human's diet, comprising administering to the human about 25 to about 200 milligrams of a compound disclosed herein, or nutraceutically acceptable derivatives thereof on a daily basis. In another aspect, about 50 milligrams of a compound of the formula I or II is administered to the human on a daily basis.

A supplement of the present invention may be ingested with or after a meal. Thus, a supplement may be taken at the time of a person's morning meal, and/or at the time of a person's noontime meal. A portion may be administered shortly before, during, or shortly after the meal. For daily consumption, a portion of the supplement may be consumed shortly before, during, or shortly after the human's morning meal, and a second portion of the supplement may be consumed shortly before, during, or shortly after the human's noontime meal. The morning portion and the noontime portion can each provide approximately the same quantity of a compound of the formula I or II. A supplement and regimens described herein may be most effective when combined with a balanced diet according to generally accepted nutritional guidelines, and a program of modest to moderate exercise several times a week.

The invention will be described in greater detail by way of specific examples. The following examples are offered for illustrative purposes, and are not intended to limit the invention in any manner.

### EXAMPLES

### Example 1

The following methods described in WO 2004/075882 (PCT/CA2004/000272) can be used to study the compounds of the invention:

Mice. Experimental groups of TgCRND8 mice [Chishti, M. A. et al., J. Biol Chem 276, 21562-21570 (2001); Janus, C. et al., Nature 408, 979-982 (2000)] will be initially treated with 5 mg/Kg/day-300 mg/Kg/day of a compound disclosed herein. Two cohorts of animals (n=10 mice per treatment arm) will be entered into the study at 6 weeks to five months of age, and outcomes will be analyzed at 4 to 6 months of age. The body weight, coat characteristics and in cage behaviour will be monitored.

Behavioural tests: Morris Water Maze testing will be performed as described in Janus, C. et al., 2000. After non-spatial pre-training, mice will undergo discrimination training for 5 days with 4-trials per day. Behavioral data will be analyzed using a mixed model of factorial analysis of variance (ANOVA) with drug or genotype and training sessions as repeated measure factors.

Cerebral amyloid burden. Brains will be removed and one hemisphere fixed in 4% paraformaldehyde and embedded in paraffin wax in the mid sagittal plane. To generate sets of systematic uniform random sections, 5 μm serial sections will be collected across the entire hemisphere. Sets of sections at 50 mm intervals will be used for analyses (10-14 sections/set). Plaques will be identified after antigen retrieval with formic acid, and incubated with primary anti-Aβ antibody (Dako M-0872), followed by secondary antibody (Dako StreptABCcomplex/horseradish kit). End products will be visualized with DAB and counter-stained with hematoxylin. Amyloid plaque burden will be assessed with Leco IA-3001 image analysis software interfaced with Leica microscope and Hitachi KP-M1U CCD video camera. Vascular amyloid burden will be similarly analyzed and a dissector will be used to measure the diameter of affected vessels.

Plasma and Cerebral Aβ Content. Hemi-brain samples will be homogenized in a buffered sucrose solution, followed by either 0.4% diethylamine/100 mM NaCl for soluble AD levels or cold formic acid for the isolation of total Aβ. After neutralization, the samples will be diluted and analyzed for Aβ40 and Aβ42 using commercially available kits (BIOSOURCE International). Each hemisphere will be analyzed in triplicate and the mean values±SEM reported. Western blot analyses will be performed on all fractions using urea gels for Aβ species analyses (Wiltfang, J. et al., J Neurochem 81, 481-496 (2002)). Aβ will be detected using 6E10 (BIOSOURCE International) and Enhanced Chemiluminenscence (Amersham). Gliosis Quantitation. Five randomly selected, evenly spaced, sagittal sections will be collected from paraformaldehyde-fixed and frozen hemispheres of treated and control mice. Sections will be immunolabelled for astrocytes with anti-rat GFAP IgG₂ₐ (Dako; diluted 1:50) and for microglia with anti-rat CD68 IgG_{2b} (Dako; 1:50). Digital images will be captured using a Coolsnap digital camera (Photometrics, Tuscon, Ariz.) mounted to a Zeiss, Axioscope 2 Plus microscope. Images will be analysed using Openlab 3.08 imaging software (Improvision, Lexington Mass.).

Survival Census: The probability of survival will be assessed by the Kaplan-Meier technique (Haccou, P., & Mellis, E., Statistical Analysis of Behavioural Data, pg 120-186, Oxford University Press, Oxford (1995)), computing the probability of survival at every occurrence of death, making it suitable for small sample sizes. The Tarone-Ware test will be used to compare the treatments.

Analysis of APP in brain. Mouse hemi-brain samples will be homogenized and spun at 109,000×g, in 20 mM Tris pH 7.4, 0.25M sucrose, 1 mM EDTA and 1 mM EGTA, and a protease inhibitor cocktail, mixed with 0.4% DEA (diethylamine)/100 mM NaCl. The supernatants will be analysed for APPs levels by Western blotting using mAb 22C11, while the pellets will be analysed for APP holoprotein with mAb C1/6.1 as described in Janus, 2000; Chishti, M, 2001.

Results

To assess their effectiveness in vivo, compounds disclosed herein will be administered to a murine model of Alzheimer's disease (TgCRND8) (Chishti, M. A. et al, J. Biol Chem 276, 21562-21570 (2001); Janus, C. et al., Nature 408, 979-982 (2000)). The TgCRND8 mice and non-transgenic littermates will be assigned to sex- and age-matched cohorts that are then used to test the effectiveness of the compounds disclosed herein as therapeutics. The mice will be randomly assigned to receive active compound, mock therapy, or no therapy. The endpoints will be cognitive function, brain Aβ levels, and neuropathology.

The data are expected to show that compounds disclosed herein can prevent and reverse the AD-like phenotype in TgCRND8 mice, reducing cognitive deficits, amyloid plaques, amyloid angiopathy, Aβ-induced inflammatory response, and/or accelerated mortality. The levels of soluble Aβ oligomers are expected to be significantly reduced in the brain of mice treated with compounds disclosed herein.

### Example 2

The compounds disclosed herein can be tested in an Alternating Lever Cyclic Ratio rat model of Alzheimer's disease (O'Hare, E. et al, Behavior Pharmacology, 7:742-753, (1996); Richardson, R L, et al., Brain Research, 54: 1-10, (2002)). This model has been able to detect cognitive deficits due to direct injection of amyloid-β oligomers into rat brain.

The compounds can be administered concurrent with Aβ oligomers known to adversely affect cognition and their ability to counteract the oligomer-induced cognitive decline can be assessed.

In the Alternativing Lever Cyclic Ratio (ALCR) test rats must first learn a complex sequence of lever-pressing requirements in order to earn food reinforcement in a two-lever experimental chamber. Subjects must alternate between two levers by switching to the other lever after pressing the first lever enough to get food rewards. The exact number of presses required for each food reward changes, first increasing from 2 responses per food pellet up to 56 based on the quadratic function, x²−x. One cycle is an entire ascending and descending sequence of these lever press requirements (e.g., 2, 6,12, 20, 30, 42, 56, 56, 42, 30, 20, 12, 6, and 2 presses per food reward). Six such full cycles are presented during each daily session. Errors can be scored when the subject perseveres on a lever after pressing enough to get the food reward, i.e., does not alternate (a Perseveration Error), or when a subject switches levers before completing the response requirement on that lever (a Switching Error).

### Example 3

Amyloid beta (Aβ) fibrils were prepared by the methods disclosed in Kheterpal, I et al, Biochemistry, 2001 40(39):11757 and Cannon M J et al, Anal Biochem. 2004 328(1):67. The fibrils were immobilized on an affinity column and assayed by FAC-MS using the methods described in Leticia Toledo-Sherman, et al, J. Med. Chem. 2005, 48: 3221 or Slon-Usakiewicz J. J. et al, Clin. Proteom. J. 2004,1:227-234. In particular, Aβ fibrils were immobilized to CBX1000C (COOH-modified) beads (Millipore) as follows. CBX1000C (5 mg) activated by reaction with EDAC/NHS in 0.1 M MES buffer containing 0.5 M NaCl, pH 6.4. After 45 min of mixing at room temperature the beads were centrifuged and supernatant was removed and washed with 1×MES. The beads were resuspended in 250 μL of MES buffer and 100 μg of Aβ fibrils (in 1×PBS) was added. The mixture was incubated for 2 h at room temperature and then overnight at 4° C. with 360° vertical rotation followed by 1×PBS. After loading immobilized Aβ fibrils, the FAC-MS capillary columns (250 μm id×2.5 cm) were washed with 50 μL (at 200 μL/h) of 1×PBS buffer followed by 50 μL of the running buffer (20 mM NH₄OAc containing 1% DMSO). The activity of the immobilized amyloid fibrils was determined using Aβ monomer (1 μM) as the indicator and M3 (1 μM) as the void marker in 20 mM NH₄OAc containing 1% DMSO. The makeup buffer was 90% methanol containing 0.1% acetic acid in water. Analyte solutions contained Aβ monomer (1 μM) as the indicator and M3 (1 μM) as the void marker and compounds (see Table 1) ranging from 1-10 μM in 20 mM NH₄OAc containing 1% DMSO. The flow rates used were 80 μL/h for the makeup buffer and 100 μL/h for the FAC-MS columns. The column was connected to an AB/Sciex API 3000 triple-quadrupole mass spectrometer (Concord, Ontario, Canada) and syringe pumps (Harvard Biosciences, Holliston, Mass.) and was allowed to equilibrate with the running buffer until the Aβ monomer (M+H) signal was stable, then data acquired. After 1 min, the system was switched to the analyte solution and data collection continued until the Aβ monomer signal had maximized for at least 10 min. The column was washed with running buffer until the Aβ monomer signal had reduced to its background level to regenerate the column. The data was analyzed using a customized Excel macro to determine the breakthrough times of amyloid beta and M3.

The % shift is determined from the equation:
% Shift=(t_{I}−t)/(t_{I}−t_{NSB})×100%
where t is the breakthrough time difference, measured at the inflection point, of the sigmoidal fronts between the indicator and void marker in the presence of any competing ligand(s), t_{NSB} is the non-specific breakthrough time difference in the absence of immobilized target (and is a constant for the indicator used) and t_{I} is the breakthrough time difference in the absence of any competing ligands.

The FAC-MS % shift results of the free Aβ monomer assayed with immobilied Aβ fibrils in the presence of various compounds at 1 and 10 μM is shown in Tables 1 and 2.

### Example 4

Mono-substituted scyllo-inositols (methyl, ethyl, benzyl, and trifluoromethyl) were synthesized as follows. A mono-methyl scyllo-inositol (9) was synthesized starting from myo-inositol (1) as described in the literature and illustrated in FIG. 1. The literature protocol for the methylation of the intermediate 6 on a 600 mg scale afforded ˜230 mg of the pure 7 and ˜300 mg of the recovered un-reacted starting material. The structure of 7 was confirmed by ¹H-NMR. 45 mg of methyl-scyllo-inositol was synthesized and identified by ¹H-NMR and MS analysis.

Alkylation of the intermediate 6 with EtI and BnBr was done on a 600 mg scale starting with 6. The products were purified by column chromatography and identified by ¹H-NMR. The intermediate 8 (Me and Bn) and the ethyl analog of 8 were also synthesized. ˜120 mg of benzyl-scyllo-inositol was synthesized and identified by ¹H-NMR and MS analysis.

Trifluoromethyl-scyllo-inositol was synthesized from intermediate 6 similar to the mono-methyl-scyllo-inositol (see FIG. 3). The fact, that trifluoroiodomethane is a gas required some modifications to the original protocol. Thus the solution of intermediate 6 in DMF was saturated with CF₃I at low temperature, then sodium hydride was added and the reaction vessel sealed. A vigorous evolution of a gas was observed, but no changes in the reaction progress were observed at low temperature.

Di-substituted scyllo-inositols (1,3-dimethyl and 1,3-diacetyl) were synthesized using a process similar to the process for producing methyl-scyllo-inositol starting from intermediate 6. A five-step reaction scheme for the synthesis of di-substituted scyllo-inositols from intermediate 6 is illustrated in FIG. 2.

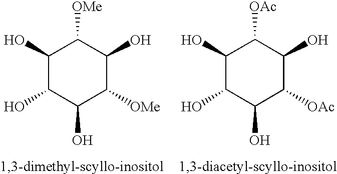

Any one or more compounds of the formula I, II, III, and IV may be excluded from any embodiment of the present invention. Compounds disclosed in Table 2 are excluded in some embodiments of the invention. In addition, compounds disclosed in WO 2004/075882 or WO 2006/053428 are excluded from the embodiments disclosed herein.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the methods etc. which are reported therein which might be used in connection with the invention. Nothing herein is to be construed as an admission that the invention is not entitled to antedate such disclosure by virtue of prior invention. 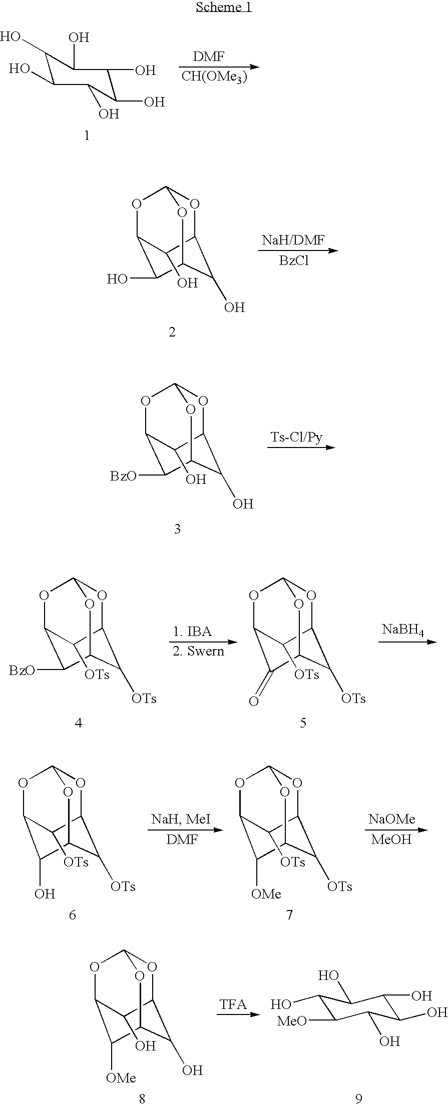
Abbreviations: CH(OCH₃)₃=trimethyl orthoformate, DMF=dimethyl formamide; NaH=sodium hydride, BzCl=benzyl chloride; IBA=iodosobenzoic acid, Swen is a Swern oxidation; NaBH₄=sodium borohydride; MeI=methyl iodide; NaOMe=sodium methoxide, MeOH=methanol; TFA is trifluoroacetic acid. 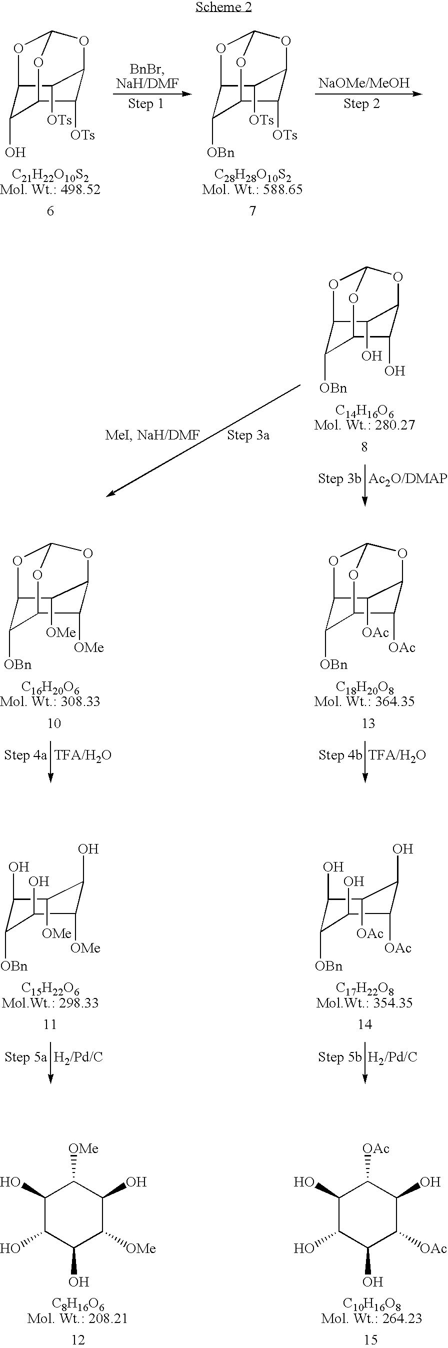
Abbreviations: Structure 6—OTs=p-toluenesulfonate; BnBr=benzyl bromide; Step 3b—DMAP=4-Dimethylaminopyridine, Ac₂O=acetic anhydride; Steps 5a, 5b—H₂/Pd/C=hydrogen and palladium on carbon; all other abbreviations as defined in FIG. 1+L.

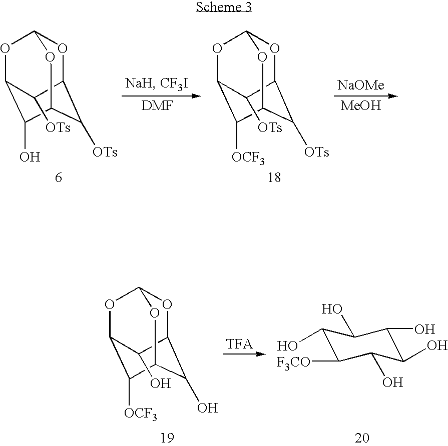

Abbreviation: CF₃I=trifluoromethyl iodide. All other abbreviations as in FIGS. 1 and 2.

TABLE I

| | | | Mol | | | |
|---|---|---|---|---|---|---|
| | Structure | Molec. | Weight | | | ABeta |
| Structure | Composition | Formula | Structure | Name | Source | Shift |
| 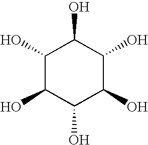 | C 40.00% H 6.71% O 53.28% | C₆H₁₂O₆ | 180.15894 | scyllo-Inositol (AZD-103) | Sigma- Aldrich | 89 |
| 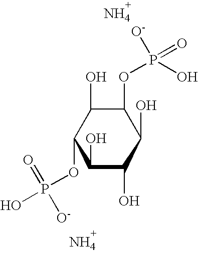 | C 19.26% H 5.39% N 7.49% O 51.31% P 16.56% | C₆H₂₀N₂O₁₂P₂ | 374.1801 | D-myo- Inositol 2,4- biphosphate ammonium salt | Sigma- Aldrich | 12 |
| 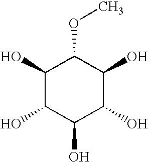 | C 43.30% H 7.27% O 46.10% | | 194.18603 | Methyl-scyllo- inositol | Dalton | 48 |
| 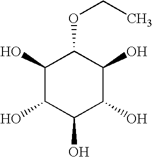 | C 46.15% H 7.37% O 48.73% | C₈H₁₆O₆ | 208.21312 | Ethyl-scyllo- inositol | Dalton | 14 |
| 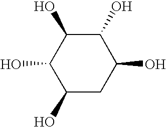 | C 43.90% H 7.37% O 48.73% | C₆H₁₂O₅ | 164.15954 | 1,3,5/2,4- pentahydroxy cyclohexane (Scyllo- Quercitol) | | 15 |
| 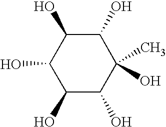 | C 43.30% H 7.27% O 49.44% | C₇H₁₄O₆ | 194.18603 | 1-Methyl- 1,3,5/2,4,6- Inositol (Mytilitol) | | 22 |
| 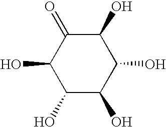 | C 40.45% H 5.66% O 53.89% | C₆H₁₀O₆ | 178.143 | 2,4,5/3,5- Pentahydroxy cyclohexanone (Scyllo- inosose) | | 11 |
| 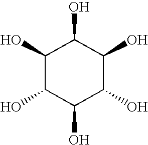 | C 40.00% H 6.71% O 53.28% | C₆H₁₂O₆ | 180.15894 | myo-Inositol | Sigma- Aldrich | 34 |
| 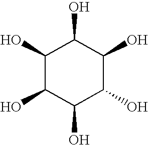 | C 40.00% H 6.71% O 53.28% | C₆H₁₂O₆ | 180.15894 | epi-Inositol | | 42 |
| 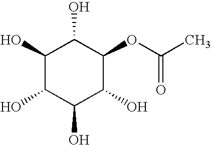 | C 43.25% H 6.35% O 50.40% | C₈H₁₄O₇ | 222.19658 | 1-acetyl- scyllo-inositol | Dalton | 31 |
| 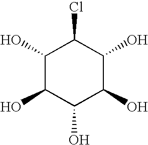 | C 36.29% H 5.58% Cl 17.85% O 40.28% | C₆H₁₁ClO₅ | 198.60457 | 1-Chloro-1- deoxy-scyllo- inositol | V-Labs | 67 |

TABLE 2

| | Structure | | ABeta |
|---|---|---|---|
| Structure | composition | SOURCE | SHIFT |
| 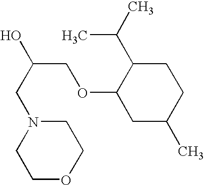 | C 68.19% H 11.11% N 4.68% O 16.03% | ASINEX | 1 |
| 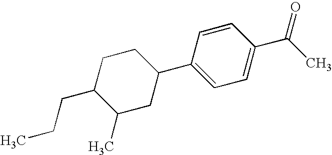 | C 83.67% H 10.14% O 6.19% | ASINEX | 1 |
| 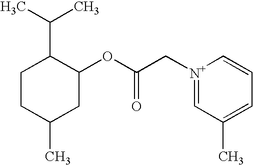 | C 74.44% H 9.72% N 4.82% O 11.02% | ASINEX | 1 |
| 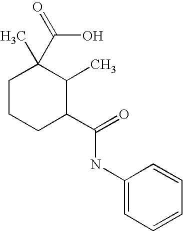 | C 69.79% H 7.69% N 5.09% O 17.43% | ASINEX | 1 |
| 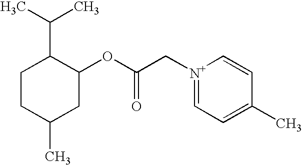 | C 74.44% H 9.72% N 4.82% O 11.02% | ASINEX | 1 |
| 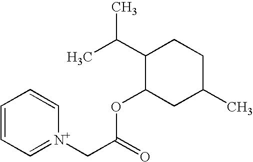 | C 73.87% H 9.48% N 5.07% O 11.58% | ASINEX | 1 |
| 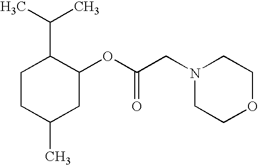 | C 67.81% H 10.31% N 4.94% O 16.94% | ASINEX | 1 |
| 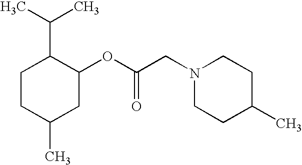 | C 73.17% H 11.26% N 4.74% O 10.83% | ASINEX | 1 |
| 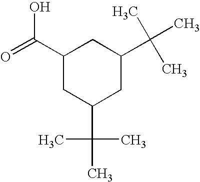 | C 74.95% H 11.74% O 13.31% | ASINEX | 1 |
| 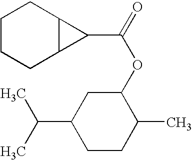 | C 77.65% H 10.86% O 11.49% | ASINEX | 1 |
| 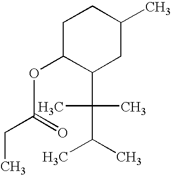 | C 75.54% H 11.89% O 12.58% | ASINEX | 1 |
| 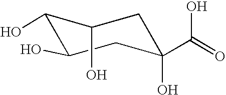 | C 43.75% H 6.29% O 49.95% | CHEMBRIDGE | 1 |
| 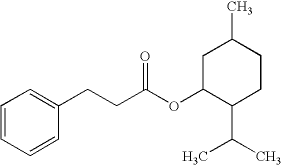 | C 79.12% H 9.79% O 11.09% | CHEMBRIDGE | 1 |
| 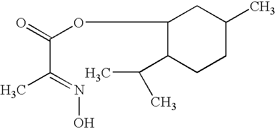 | C 64.70% H 9.61% N 5.80% O 19.89% | CHEMBRIDGE | 1 |
| 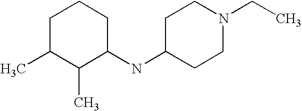 | C 75.57% H 12.68% N 11.75% | CHEMBRIDGE | 1 |
| 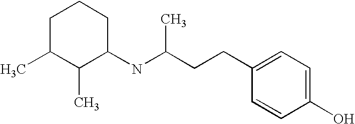 | C 78.49% H 10.61% N 5.09% O 5.81% | CHEMBRIDGE | 1 |
| 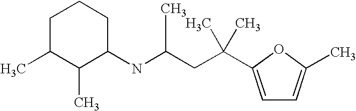 | C 78.29% H 11.41% N 4.81% O 5.49% | CHEMBRIDGE | 1 |
| 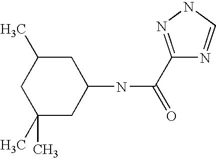 | C 60.99% H 8.53% N 23.71% O 6.77% | CHEMBRIDGE | 1 |
| 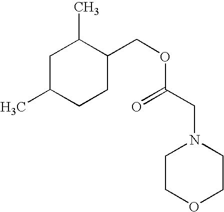 | C 66.88% H 10.10% N 5.20% O 17.82% | CHEMBRIDGE | 1 |
| 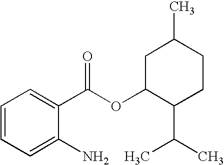 | C 74.14% H 9.15% N 5.09% O 11.62% | CHEMBRIDGE | 1 |
| 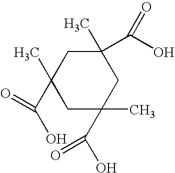 | C 55.81% H 7.03% O 37.17% | CHEMBRIDGE | 1 |
| 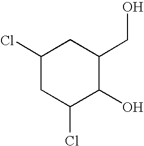 | C 42.23% H 6.08% Cl 35.62% O 16.07% | SPECS | 0 |
| 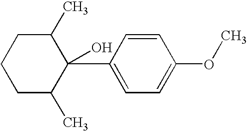 | C 76.88% H 9.67% O 13.65% | SPECS | 0 |
| 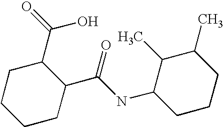 | C 68.29% H 9.67% N 4.98% O 17.06% | SPECS | 0 |
| 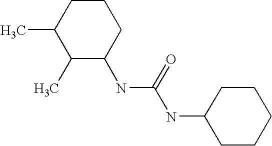 | C 71.38% H 11.18% N 11.10% O 6.34% | SPECS | 0 |
| 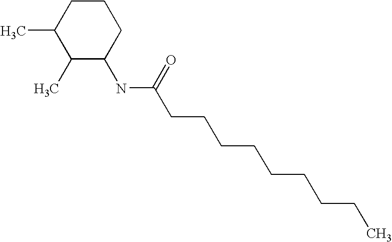 | C 76.81% H 12.53% N 4.98% O 5.68% | SPECS | 0 |
| 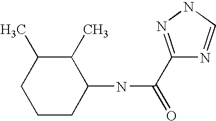 | C 59.44% H 8.16% N 25.20% O 7.20 | SPECS | 0 |
| 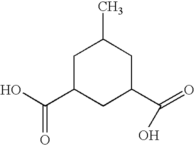 | C 58.05% H 7.58% O 34.37% | SPECS | 0 |
| 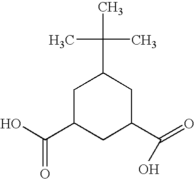 | C 63.14% H 8.83% O 28.03% | SPECS | 0 |
| 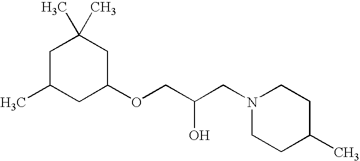 | C 72.68% H 11.86% N 4.71% O 10.76% | SPECS | 0 |
| 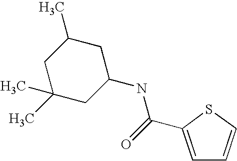 | C 66.89% H 8.42% N 5.57% O 6.36% S 12.75% | SPECS | 0 |
| 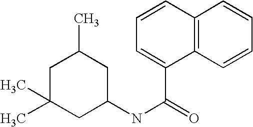 | C 81.31% H 8.53% N 4.74% O 5.42% | SPECS | 0 |
| 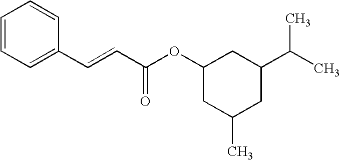 | C 79.68% H 9.15% O 11.17% | SPECS | 0 |
| 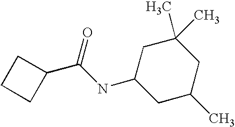 | C 75.28% H 11.28% N 6.27% O 7.16% | SPECS | 0 |
| 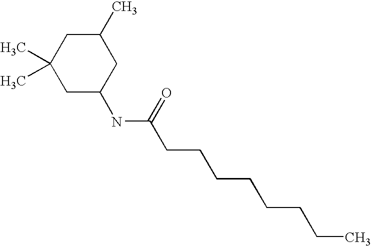 | C 76.81% H 4.98% N 4.98% O 5.68% | SPECS | 0 |
| 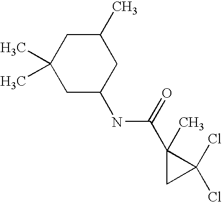 | C 57.54% H 7.93% Cl 24.26% N 4.79% O 5.47% | SPECS | 0 |
| 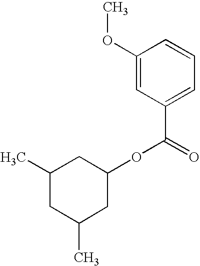 | C 73.25% H 8.45% O 18.30% | SPECS | 0 |
| 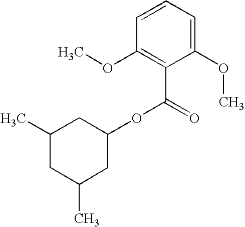 | C 69.84% H 8.27% O 21.89% | SPECS | 0 |
| 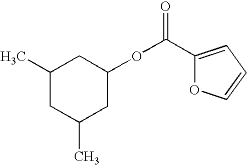 | C 70.24% H 8.16% O 21.59% | SPECS | 0 |
| 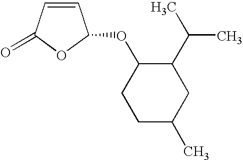 | C 70.56% H 9.30% O 20.14% | SPECS | 0 |
| 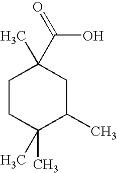 | C 71.70% H 10.94% O 17.36% | SPECS | 0 |
| 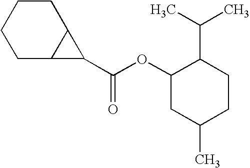 | C 77.65% H 10.86% O 11.49% | SPECS | 0 |
| 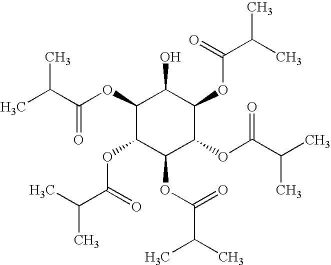 | C 58.85% H 7.98% O 33.17% | SPECS | 4 |
| 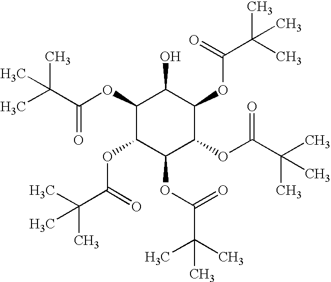 | C 61.98% H 8.73% O 29.30% | SPECS | 5 |
| 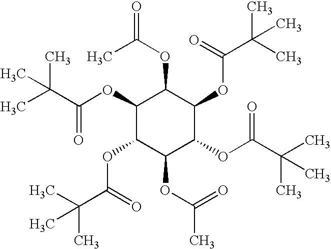 | C 59.98% H 8.05% O 31.96% | SPECS | 4 |
| 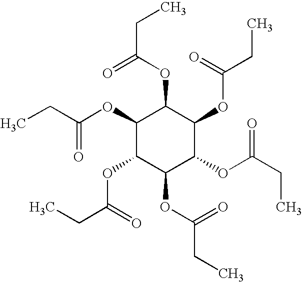 | C 55.81% H 7.03% O 37.17% | SPECS | 4 |
| 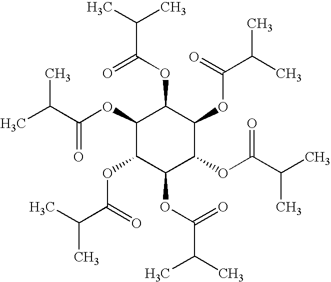 | C 59.98% H 8.05% O 31.96% | SPECS | 5 |
| 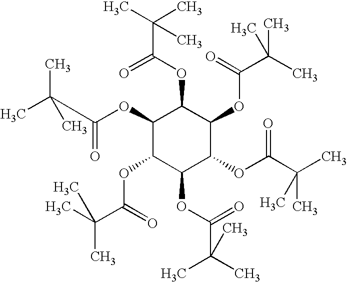 | C 63.14% H 8.83% O 28.03% | SPECS | 4 |
| 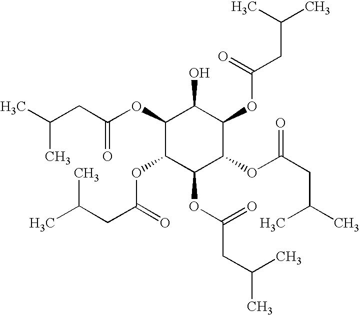 | C 61.98% H 8.73% O 29.30% | SPECS | 2 |
| 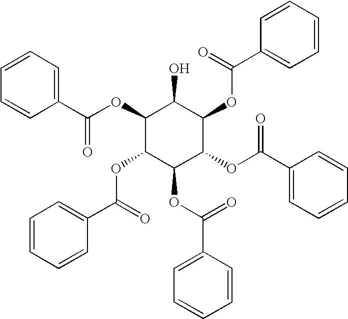 | C 70.28% H 4.60% O 25.12% | SPECS | 2 |
| 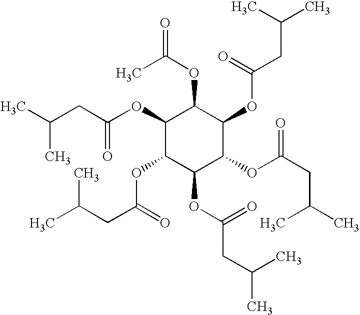 | C 61.66% H 8.47% O 29.87% | SPECS | 3 |
| 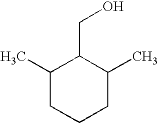 | C 76.00% H 12.76% O 11.25% | ASDI | 1 |
| 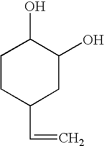 | C 67.57% H 9.92% O 22.50% | ASDI | 1 |
| 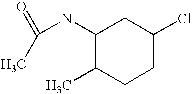 | C 56.99% H 8.50% Cl 18.69% N 7.38% O 8.43% | ASDI | 1 |
| 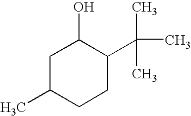 | C 77.58% H 13.02% O 9.39% | ASDI | 1 |
| 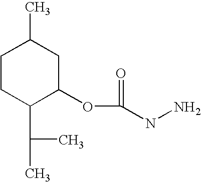 | C 61.65% H 10.35% N 13.07% O 14.93% | ASDI | 1 |
| 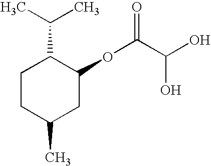 | C 62.58% H 9.63% O 27.79% | ASDI | 1 |
| 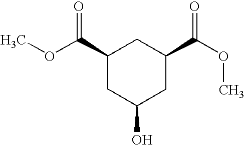 | C 55.55% H 7.46% O 37.00% | ASDI_PRIM | 1 |
| 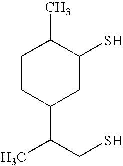 | C 58.76% H 9.86% S 31.37% | ASDI | 1 |
| 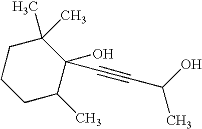 | C 74.24% H 10.54% O 15.21% | ASDI | 1 |
| 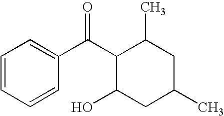 | C 77.55% H 8.68% O 13.77% | ASDI | 1 |
| 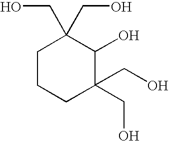 | C 54.53% H 9.15% O 36.32% | ASDI | 1 |
| 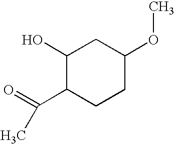 | C 62.77% H 9.36% O 27.87% | ASDI | 1 |
| 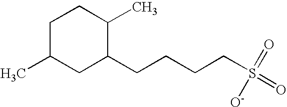 | C 53.31% H 8.57% Na 8.50% O 17.75% S 11.86% | ASDI | 1 |
| 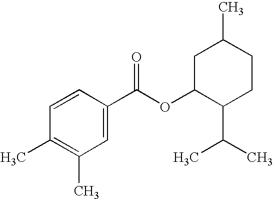 | C 79.12% H 9.79% O 11.09% | CHEMDIV | 2 |
| 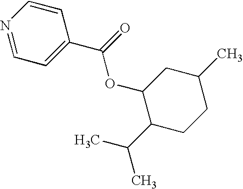 | C 73.53% H 8.87% N 5.36% O 12.24% | CHEMDIV | 1 |
| 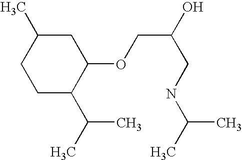 | C 70.80% H 12.25% N 5.16% O 11.79% | CHEMDIV | 1 |
| 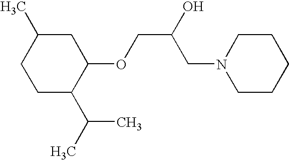 | C 72.68% H 11.86% N 4.71% O 10.76% | CHEMDIV | 1 |
| 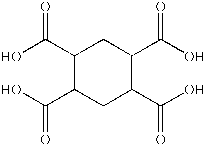 | C 46.16% H 4.65% O 49.19% | CHEMDIV | 1 |
| 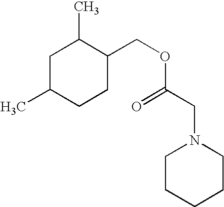 | C 71.87% H 10.93% N 5.24% O 11.97% | CHEMDIV | 1 |
| 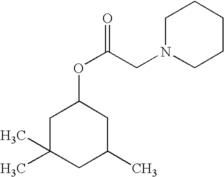 | C 71.87% H 10.93% N 5.24% O 11.97% | CHEMDIV | 1 |
| 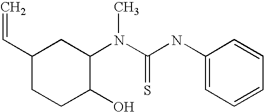 | C 66.17% H 7.64% N 9.65% O 5.51% S 11.04 | CHEMDIV | 1 |
| 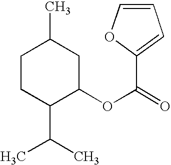 | C 71.97% H 8.86% O 19.17% | CHEMDIV | 2 |
| 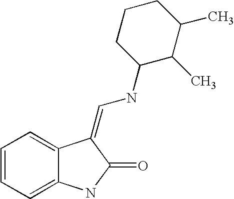 | C 75.52% H 8.20% N 10.36% O 5.92% | CHEMDIV | 2 |
| 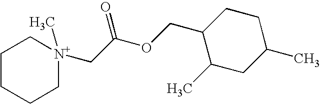 | C 72.29% H 11.42% N 4.96% O 11.33% | CHEMDIV | 2 |
| 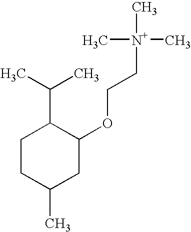 | C 74.32% H 13.31% N 5.78% O 6.60% | CHEMDIV | 2 |
| 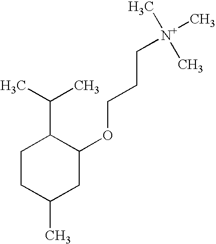 | C 74.94% H 13.36% N 5.46% O 6.24% | CHEMDIV | 2 |
| 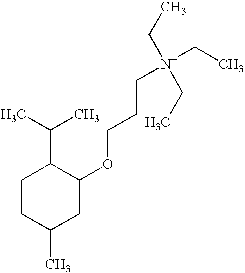 | C 76.44% H 13.51% N 4.69% O 5.36% | CHEMDIV | 2 |
| 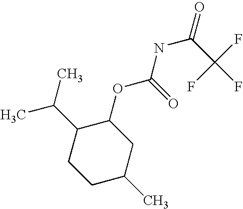 | C 52.88% H 6.83% F 19.30% N 4.74% O 16.25% | CHEMDIV | 2 |
| 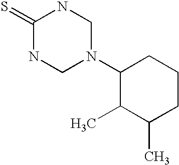 | C 58.11% H 9.31% N 18.48% S 14.10% | CHEMDIV | 2 |
| 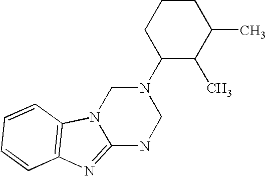 | C 71.79% H 8.51% N 19.70% | CHEMDIV | 2 |
| 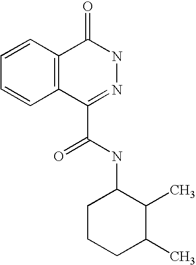 | C 68.21% H 7.07% N 14.04% O 10.69% | CHEMDIV | 2 |
| 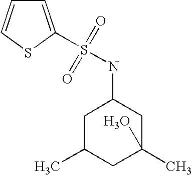 | C 54.32% H 7.36% N 4.87% O 11.13% S 22.31% | CHEMDIV | 2 |
| 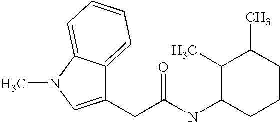 | C 76.47% H 8.78% N 9.39% O 5.36% | CHEMDIV | 2 |
| 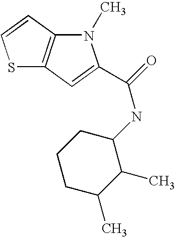 | C 66.17% H 7.64% N 9.65% O 5.51% S 11.04% | CHEMDIV | 2 |
| 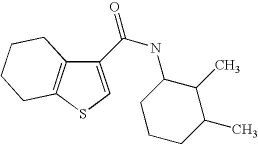 | C 70.06% H 8.65% N 4.81% O 5.49% S 11.00% | CHEMDIV | 2 |
| 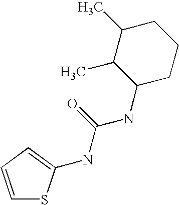 | C 61.87% H 7.99% N 11.10% O 6.34% S 12.70% | CHEMDIV | 2 |
| 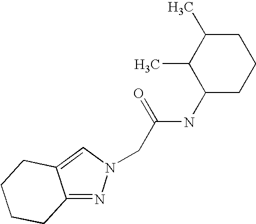 | C 70.55% H 9.40% N 14.52% O 5.53% | CHEMDIV | 1 |
| 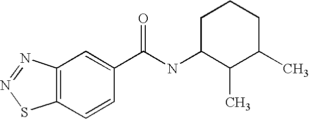 | C 62.25% H 6.62% N 14.52% O 5.53% S 11.08% | CHEMDIV | 1 |
| 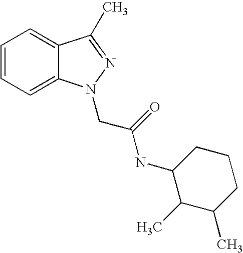 | C 72.21% H 8.42% N 14.03% O 5.34% | CHEMDIV | 1 |
| 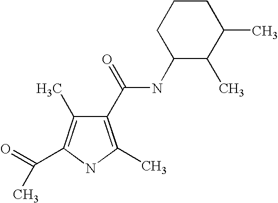 | C 70.31% H 9.02% N 9.65% O 11.02% | CHEMDIV | 1 |
| 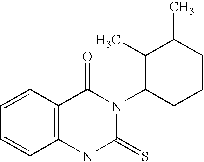 | C 66.63% H 6.99% N 9.71% O 5.55% S 11.12% | CHEMDIV | 1 |
| 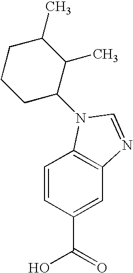 | C 70.56% H 7.40% N 10.29% O 11.75% | CHEMDIV | 1 |
| 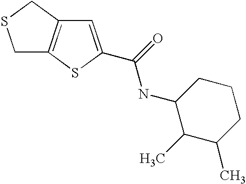 | C 60.98% H 7.16% N 4.74% O 5.41% S 21.70% | CHEMDIV | 1 |
| 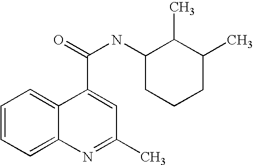 | C 76.99% H 8.16% N 9.45% O 5.40% | CHEMDIV | 1 |
| 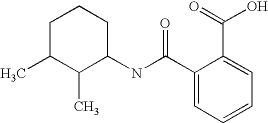 | C 69.79% H 7.69% N 5.09% O 17.43% | CHEMDIV | 2 |
| 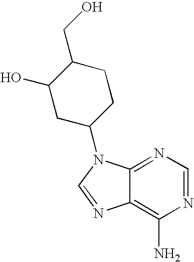 | C 54.74% H 6.51% N 26.60% O 12.15% | ENAMINE | 1 |
| 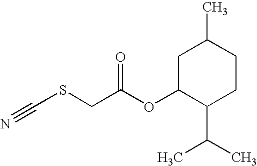 | C 61.14% H 8.29% N 5.48% O 12.53% S 12.56% | ENAMINE | 1 |
| 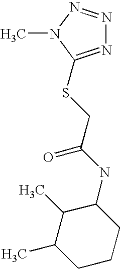 | C 50.86% H 7.47% N 24.71% O 5.65% S 11.31% | ENAMINE | 1 |
| 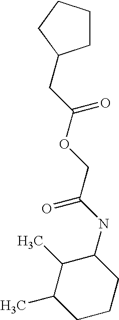 | C 69.12% H 9.89% N 4.74% O 16.25% | ENAMINE | 1 |
| 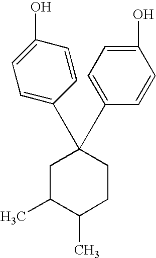 | C 81.04% H 8.16% O 10.80% | ENAMINE | 1 |
| 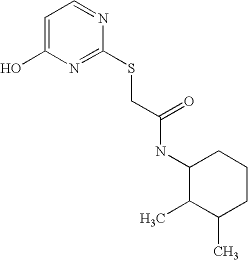 | C 56.92% H 7.17% N 14.22% O 10.83% S 10.85% | ENAMINE | 1 |
| 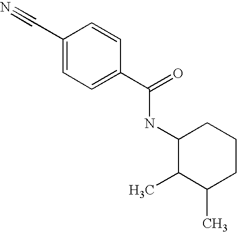 | C 74.97% H 7.86% N 10.93% O 6.24% | ENAMINE | 1 |
| 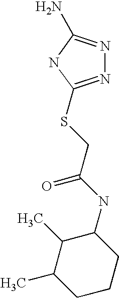 | C 50.86% H 7.47% N 24.71% O 5.65% S 11.31% | ENAMINE | 1 |
| 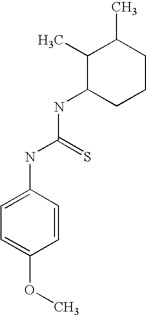 | C 65.71% H 8.27% N 9.58% O 5.47% S 10.96% | ENAMINE | 1 |
| 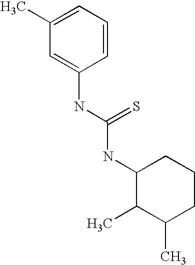 | C 69.52% H 8.75% N 10.13% S 11.60% | ENAMINE | 1 |
| 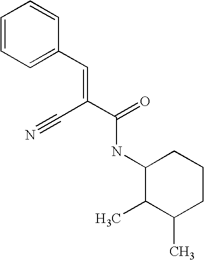 | C 76.56% H 7.85% N 9.92% O 5.67% | ENAMINE | 1 |
| 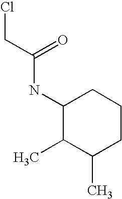 | C 58.96% H 8.91% Cl 17.40% N 6.88% O 7.85% | ENAMINE | 1 |
| 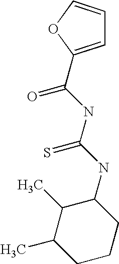 | C 59.97% H 7.19% N 9.99% O 11.41% S 11.44% | ENAMINE | 1 |
| 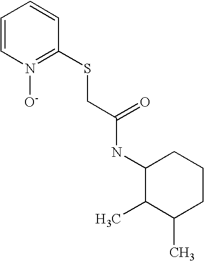 | C 61.19% H 7.53% N 9.51% O 10.87% S 10.89% | ENAMINE | 1 |
| 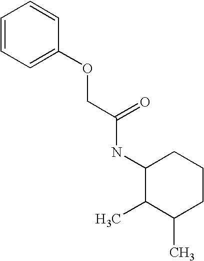 | C 73.53% H 8.87% N 5.36% O 12.24% | ENAMINE | 1 |
| 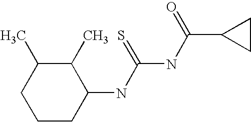 | C 61.38% H 8.72% N 11.01% O 6.29% S 12.60% | ENAMINE | 1 |
| 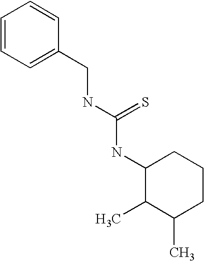 | C 69.52% H 8.75% N 10.13% S 11.60% | ENAMINE | 1 |
| 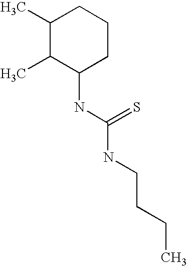 | C 64.41% H 10.81% N 11.56% S 13.23% | ENAMINE | 1 |
| 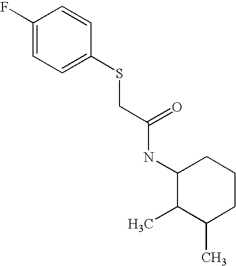 | C 65.05% H 7.51% F 6.43% N 4.74% O 5.42% S 10.85% | ENAMINE | 1 |
| 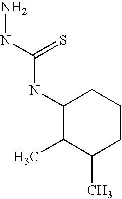 | C 53.69% H 9.51% N 20.87% S 15.93% | ENAMINE | 1 |
| 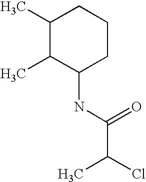 | C 60.68% H 9.26% Cl 16.28% N 6.43% O 7.35% | ENAMINE | 1 |
| 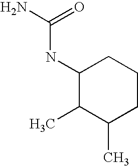 | C 63.49% H 10.66% N 16.45% O 9.40% | ENAMINE | 1 |
| 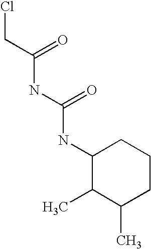 | C 53.55% H 7.76% Cl 14.37% N 11.35% O 12.97% | ENAMINE | 1 |
| 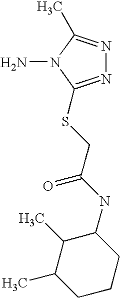 | C 52.50% H 7.79% N 23.55% O 5.38% S 10.78% | ENAMINE | 1 |
| 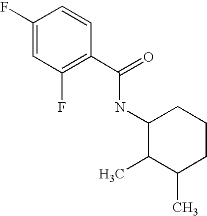 | C 67.40% H 7.16% F 14.21% N 5.24% O 5.99% | ENAMINE | 1 |
| 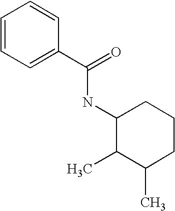 | C 77.88% H 9.15% N 6.05% O 6.92% | ENAMINE | 1 |
| 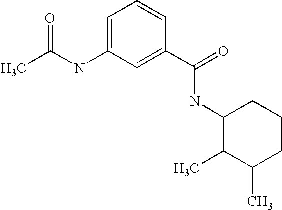 | C 70.80% H 8.39% N 9.71% O 11.10% | ENAMINE | 1 |
| 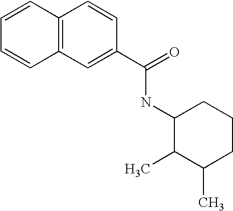 | C 81.10% H 8.24% N 4.98% O 5.69% | ENAMINE | 1 |
| 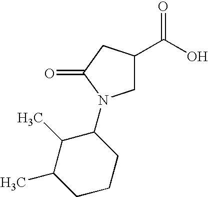 | C 65.25% H 8.85% N 5.85% O 20.06% | ENAMINE | 1 |
| 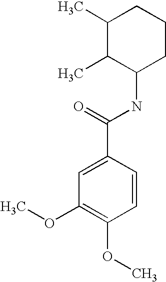 | C 70.07% H 8.65% N 4.81% O 16.47% | ENAMINE | 1 |
| 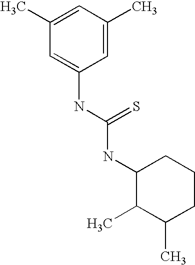 | C 70.30% H 9.02% N 9.64% S 11.04% | ENAMINE | 1 |
| 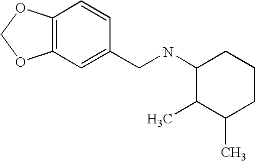 | C 73.53% H 8.87% N 5.36% O 12.24% | ENAMINE | 1 |
| 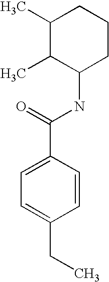 | C 78.72% H 9.71% N 5.40% O 6.17% | ENAMINE | 1 |
| 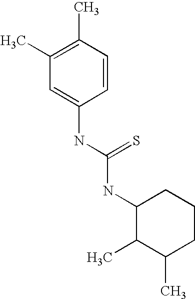 | C 70.30% H 9.02% N 9.64% S 11.04% | ENAMINE | 1 |
| 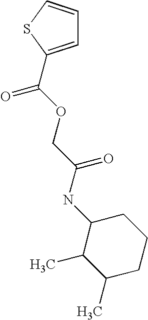 | C 60.99% H 7.17% N 4.74% O 16.25% S 10.85% | ENAMINE | 1 |
| 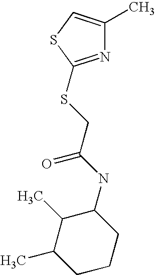 | C 56.34% H 7.43% N 9.39% O 5.36% S 21.49% | ENAMINE | 1 |
| 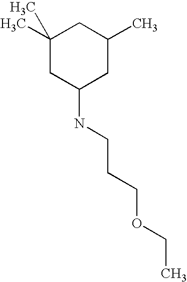 | C 73.95% H 12.86% N 6.16% O 7.04% | ENAMINE | 1 |
| 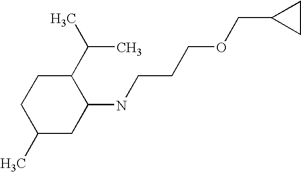 | C 76.34% H 12.44% N 5.24% O 5.98% | ENAMINE | 1 |
| 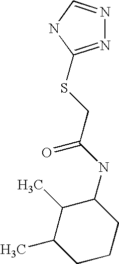 | C 53.70% H 7.51% N 20.88% O 5.96% S 11.95& | ENAMINE | 1 |
| 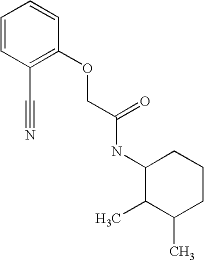 | C 71.30% H 7.74% N 9.78% O 11.17% | ENAMINE | 1 |
| 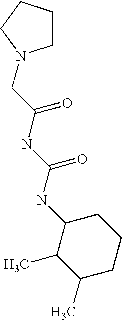 | C 64.03% H 9.67% N 14.93% O 11.37% | ENAMINE | 1 |
| 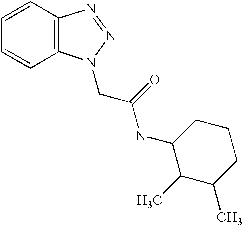 | C 67.11% H 7.74% N 19.56% O 5.59% | ENAMINE | 1 |
| 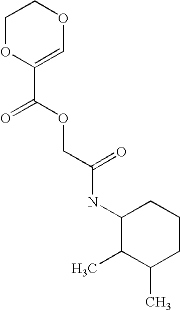 | C 60.59% H 7.80% N 4.71% O 26.90% | ENAMINE | 1 |
| 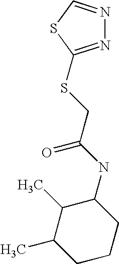 | C 50.50% H 6.71% N 14.72% O 5.61% S 22.47% | ENAMINE | 1 |
| 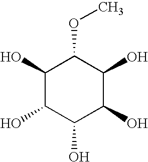 | C 43.30% H 7.27% O 49.44% | SIGMA- ALDRICH | 3 |
| 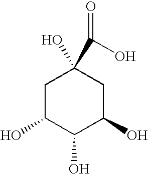 | C 43.75% H 6.29% O 49.95% | SIGMA- ALDRICH | 3 |
| 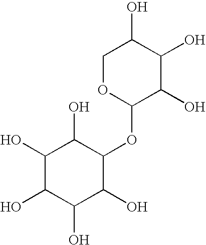 | C 42.31% H 6.46% O 51.23% | SIGMA- ALDRICH | 3 |
| 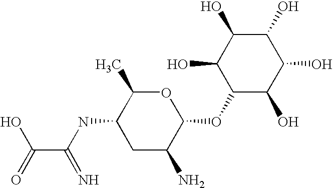 | C 42.32% H 6.85% N 10.57% O 40.26% | SIGMA- ALDRICH | 2 |
| 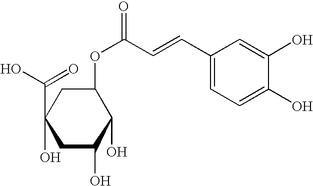 | C 54.24% H 5.12% O 40.64% | SIGMA- ALDRICH | 2 |
| 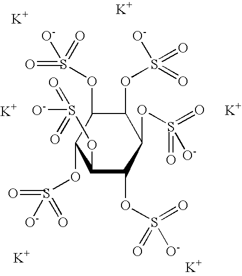 | C 8.11% H 0.68% K 26.39% O 43.19% S 21.64% | SIGMA- ALDRICH | 4 |
| 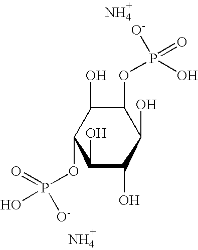 | C 19.26% H 5.39% N 7.49% O 51.31% P 16.56% | SIGMA- ALDRICH | 12 |
| 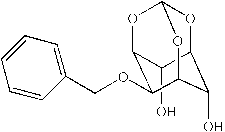 | C 60.00% H 5.75% O 34.25% | AMRI | 3 |
| 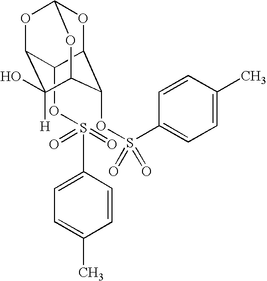 | C 50.60% H 4.45% O 32.09% S 12.86% | AMRI | 3 |
| 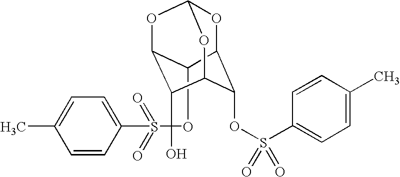 | C 50.60% H 4.45% O 32.09% S 12.86% | AMRI | 4 |
| 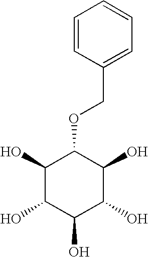 | C 57.77% H 6.71% O 35.52% | DALTON | 3 |
| 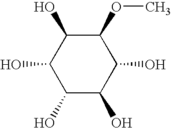 | C 43.30% H 7.27% O 49.44% | | 5 |
| 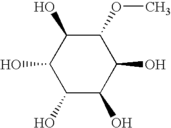 | C 43.30% H 7.27% O 49.44% | | 2 |
| 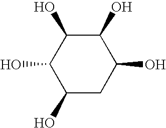 | C 43.90% H 7.37% O 48.73% | | 4 |
| 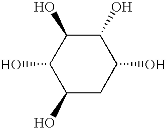 | C 43.90% H 7.37% O 48.73% | | 5 |
| 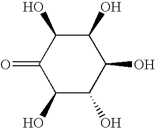 | C 40.45% H 5.66% O 53.89% | | 7 |
| 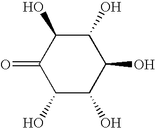 | C 40.45% H 5.66% O 53.89% | | 5 |
| 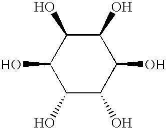 | C 40.00% H 6.71% O 53.28% | | 10 |
| 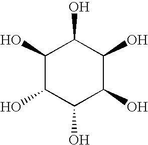 | C 40.00% H 6.71% O 53.28% | SIGMA- ALDRICH | 4 |
| 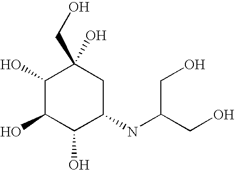 | C 44.94% H 7.92% N 5.24% O 41.90% | MOLCAN | 3 |
| 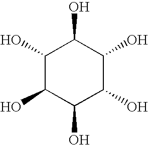 | C 40.00% H 6.71% O 53.28% | IRL | 2 |
| 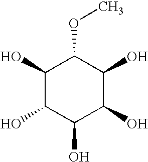 | C 43.30% H 7.27% O 49.44% | IRL | 4 |
| 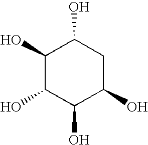 | C 43.90% H 7.37% O 48.73% | IRL | 2 |
| 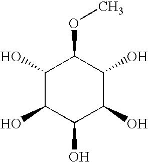 | C 43.30% H 7.27% O 49.44% | IRL | 3 |
| 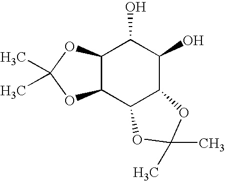 | C 55.37% H 7.75% O 36.88% | IRL | 2 |
| 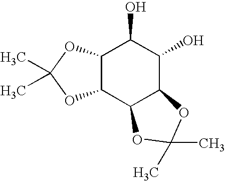 | C 55.37% H 7.75% O 36.88% | IRL | 2 |
| 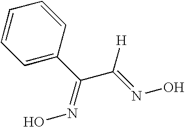 | C 58.53% H 4.91% N 17.06% O 19.49% | ChemDiv | 0 |
| 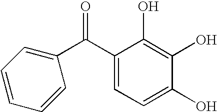 | C 67.82% H 4.38% O 27.80% | ChemDiv | 0 |
| 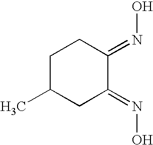 | C 53.83% H 7.74% N 17.94% O 20.49% | ChemDiv | 0 |
| 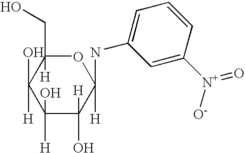 | C 48.00% H 5.37% N 9.33% O 37.30% | ChemDiv | 0 |
| 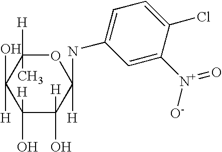 | C 45.22% H 4.74% Cl 11.12% N 8.79% O 30.12% | ChemDiv | 0 |
| 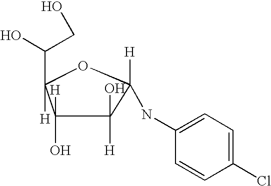 | C 49.75% H 5.57% Cl 12.24% N 4.83% O 27.61% | ChemDiv | 0 |
| 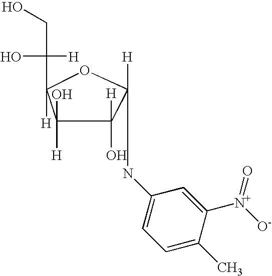 | C 49.68% H 5.77% N 8.91% O 35.63% | ChemDiv | 0 |
| +00 | | | |
| 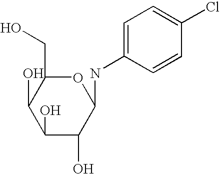 | C 49.75% H 5.57% Cl 12.24% N 4.83% O 27.61% | ChemDiv | 0 |
| 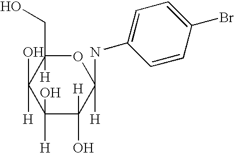 | C 43.13% H 4.83% Br 23.91% N 4.19% O 23.94% | ChemDiv | 0 |
| 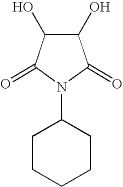 | C 56.33% H 7.09% N 6.57% O 30.01% | ChemDiv | 0 |
| 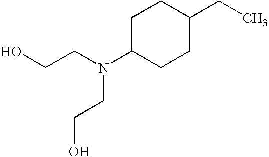 | C 66.93% H 11.70% N 6.50% O 14.86% | ChemBridge | 2 |
| 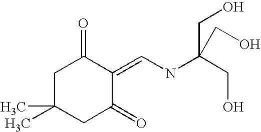 | C 57.55% H 7.80% N 5.16% O 29.48% | ChemBridge | 2 |
| 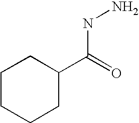 | C 59.13% H 9.92% N 19.70% O 11.25% | Timtec | 2 |
| 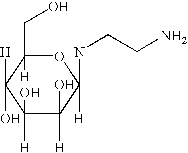 | C 43.24% H 8.16% N 12.60% O 36.00% | Timtec | 2 |
| 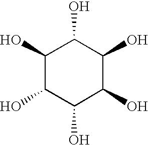 | C 40.00% H 6.71% O 53.28% | SIGMA- ALDRICH | 2 |
| 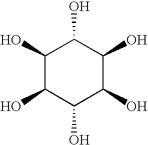 | C 40.00% H 6.71% O 53.28% | SIGMA- ALDRICH | 4 |
| 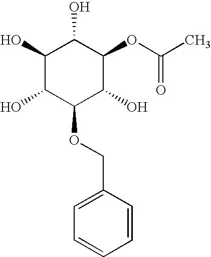 | C 57.69% H 6.45% O 35.86% | DALTON | 1 |
| 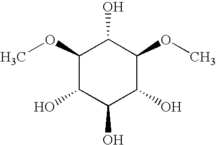 | C 46.15% H 7.75% O 46.10% | DALTON | 3 |
| 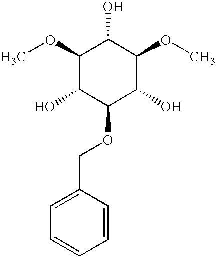 | C 60.39% H 7.43% O 32.18% | DALTON | 2 |

What is claimed is:

1. A pharmaceutical composition for treating a disease characterized by abnormal protein folding or aggregation or amyloid formation, deposition, accumulation or persistence in a subject comprising a therapeutically effective amount of a compound of the formula III, 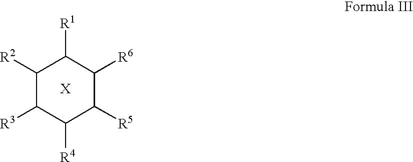
wherein X is a cyclohexane ring, where
at least one of R¹, R², R³, R⁴, R⁵, and R⁶ is independently selected from hydrogen, C₁-C₆ alkyl, C₂-C₆ alkenyl, C₂-C₆ alkynyl, C₁-C₆ alkoxy, C₂-C₆ alkenyloxy, C₃-C₁₀ cycloalkyl, C₄-C₁₀ cycloalkenyl, C₃-C₁₀ cycloalkoxy, C₆-C₁₀ aryl, C₆-C₁₀ aryloxy, C₆-C₁₀ aryl-C₁-C₃alkoxy, C₆-C₁₀ aroyl, C₆-C₁₀ heteroaryl, C₃-C₁₀ heterocyclic, C₁-C₆ acyl, C₁-C₆ acyloxy, —NH₂, —NHR⁷, —NR⁷R⁸, ═NR⁷, —S(O)₀₋₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and S(O)₂NR⁷R⁸ wherein R⁷ and R³ are independently selected from C₁-C₆ alkyl, C₂-C₆ alkenyl, C₂-C₆ alkynyl, C₃-C₁₀ cycloalkyl, C₄-C₁₀ cycloalkenyl, C₆-C₁₀ aryl, C₆-C₁₀ aryl C₁-C₃ alkyl, C₆-C₁₀ heteroaryl and C₃-C₁₀ heterocyclic; and
at least one of the remainder of R¹, R², R³, R⁴, R⁵, or R⁶ is hydroxyl, or
a pharmaceutically acceptable salt thereof,
and a pharmaceutically acceptable carrier, excipient, or vehicle.

2. The pharmaceutical composition according to claim 1 comprising a therapeutically effective amount of a compound of the formula IV, 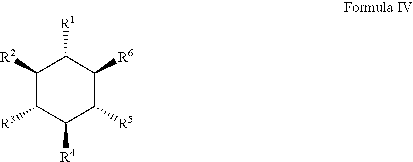
wherein R¹, R², R³, R⁴, R⁵, and R⁶ are defined as in claim 1, or a pharmaceutically acceptable salt thereof;
and a pharmaceutically acceptable carrier, excipient, or vehicle.

3. The pharmaceutical composition according to claim 1, where
R² is hydroxyl; and
R¹, R³, R⁴, R⁵, and R⁶ are independently selected from C₁-C₆ alkyl, C₂-C₆ alkenyl, C₂-C₆ alkynyl, C₁-C₆ alkoxy, C₂-C₆ alkenyloxy, C₃-C₁₀ cycloalkyl, C₄-C₁₀ cycloalkenyl, C₃-C₁₀ cycloalkoxy, C₆-C₁₀ aryl, C₆-C₁₀ aryloxy, C₆-C₁₀ aryl-C₁-C₃ alkoxy, C₆-C₁₀ aroyl, C₆-C₁₀ heteroaryl, C₃-C₁₀ heterocyclic, C₁-C₆ acyl, C₁-C₆ acyloxy, hydroxyl, —NH₂, —NHR⁷, —NR⁷R⁸—, ═NR⁷, —S(O)₀₋₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆ alkenyl, C₂-C₆ alkynyl, C₃-C₁₀ cycloalkyl, C₄-C₁₀ cycloalkenyl, C₆-C₁₀ aryl, C₆-C₁₀ aryl C₁-C₃ alkyl, C₆-C₁₀ heteroaryl and C₃-C₁₀ heterocyclic;
provided that R¹, R², R³, R⁴, R⁵, and R⁶ are not all hydroxyl;
and a pharmaceutically acceptable carrier, excipient, or vehicle.

4. The pharmaceutical composition according to claim 1, where
R² is hydroxyl;
one of R¹, R³, R⁴, R⁵, and R⁶ is hydroxyl; and
four of R¹, R³, R⁴, R⁵, and R⁶ are independently selected from C₁-C₆ alkyl, C₂-C₆ alkenyl, C₂-C₆ alkynyl, C₁-C₆ alkoxy, C₂-C₆ alkenyloxy, C₃-C₁₀ cycloalkyl, C₄-C₁₀ cycloalkenyl, C₃-C₁₀ cycloalkoxy, C₆-C₁₀ aryl, C₆-C₁₀ aryloxy, C₆-C₁₀ aryl-C₁-C₃ alkoxy, C₆-C₁₀ aroyl, C₆-C₁₀ heteroaryl, C₃-C₁₀ heterocyclic, C₁-C₆ acyl, C₁-C₆ acyloxy, —NH₂, —NHR⁷, —NR⁷R⁸—, ═NR⁷, —S(O)₀₋₂R⁷, —SH, —S₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆ alkenyl, C₂-C₆ alkynyl, C₃-C₁₀ cycloalkyl, C₄-C₁₀ cycloalkenyl, C₆-C₁₀ aryl, C₆-C₁₀ aryl C₁-C₃ alkyl, C₆-C₁₀ heteroaryl and C₃-C₁₀ heterocyclic;
and a pharmaceutically acceptable carrier, excipient, or vehicle.

5. The pharmaceutical composition according to claim 1, where
R² is hydroxyl;
two of R¹, R³, R⁴, R⁵, and R⁶ are hydroxyl; and
three of R¹, R³, R⁴, R⁵, and R⁶ are independently selected from C₁-C₆ alkyl, C₂-C₆ alkenyl, C₂-C₆ alkynyl, C₁-C₆ alkoxy, C₂-C₆ alkenyloxy, C₃-C₁₀ cycloalkyl, C₄-C₁₀ cycloalkenyl, C₃-C₁₀ cycloalkoxy, C₆-C₁₀ aryl, C₆-C₁₀ aryloxy, C₆-C₁₀ aryl-C₁-C₃ alkoxy, C₆-C₁₀ aroyl, C₆-C₁₀ heteroaryl, C₃-C₁₀ heterocyclic, C₁-C₆ acyl, C₁-C₆ acyloxy, —NH₂, —NHR⁷, —NR⁷R⁸—, ═NR⁷, —S(O)₀₋₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, C(O)NR⁷R⁸, —NHS(O)₂R⁷, —S(O)₂N H₂, —S(O)₂N H R⁷, and —S(O)₂NR⁷R⁸ wherein R⁷ and R⁸ are independently selected from C₁-C₆ alkyl, C₂-C₆ alkenyl, C₂-C₆ alkynyl, C₃-C₁₀ cycloalkyl, C₄-C₁₀ cycloalkenyl, C₆-C₁₀ aryl, C₆-C₁₀ aryl C₁-C₃ alkyl, C₆-C₁₀ heteroaryl and C₃-C₁₀ heterocyclic;
and a pharmaceutically acceptable carrier, excipient, or vehicle.

6. The pharmaceutical composition according to claim 1, where
R² is hydroxyl;
three of R¹, R³, R⁴, R⁵, and R⁶ is hydroxyl; and
two of R¹, R³, R⁴, R⁵, and R⁶ are independently selected from C₁-C₆ alkyl, C₂-C₆ alkenyl, C₂-C₆ alkynyl, C₁-C₆ alkoxy, C₂-C₆ alkenyloxy, C₃-C₁₀ cycloalkyl, C₄-C₁₀ cycloalkenyl, C₃-C₁₀ cycloalkoxy, C₆-C₁₀ aryl, C₆-C₁₀ aryloxy, C₆-C₁₀ aryl-C₁-C₃ alkoxy, C₆-C₁₀ aroyl, C₆-C₁₀ heteroaryl, C₃-C₁₀ heterocyclic, C₁-C₆ acyl, C₁-C₆ acyloxy, —NH₂, —NHR⁷, —NR⁷R⁸—, ═NR⁷, —S(O)₀₋₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆ alkenyl, C₂-C₆ alkynyl, C₃-C₁₀ cycloalkyl, C₄-C₁₀ cycloalkenyl, C₆-C₁₀ aryl, C₆-C₁₀ aryl C₁-C₃alkyl, C₆-C₁₀ heteroaryl and C₃-C₁₀ heterocyclic;
and a pharmaceutically acceptable carrier, excipient, or vehicle.

7. The pharmaceutical composition according to claim 1, where
R² is hydroxyl;
four of R¹, R³, R⁴, R⁵, and R⁶ are hydroxyl; and
one of R¹, R³, R⁴, R⁵, and R⁶ are independently selected from C₁-C₆ alkyl, C₂-C₆ alkenyl, C₂-C₆ alkynyl, C₁-C₆ alkoxy, C₂-C₆ alkenyloxy, C₃-C₁₀ cycloalkyl, C₄-C₁₀ cycloalkenyl, C₃-C₁₀ cycloalkoxy, C₆-C₁₀ aryl, C₆-C₁₀ aryloxy, C₆-C₁₀ aryl-C₁-C₃ alkoxy, C₆-C₁₀ aroyl, C₆-C₁₀ heteroaryl, C₃-C₁₀ heterocyclic, C₁-C₆ acyl, C₁-C₆ acyloxy, —NH₂, —NHR⁷, —NR⁷R⁸—, ═NR⁷, —S(O)₀₋₂R⁷, —SH, —SO₃H, nitro, cyano, halo, haloalkyl, haloalkoxy, hydroxyalkyl, —Si(R⁷)₃, —OSi(R⁷)₃, —CO₂H, —CO₂R⁷, oxo, —PO₃H, —NHC(O)R⁷, —C(O)NH₂, —C(O)NHR⁷, C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆ alkenyl, C₂-C₆ alkynyl, C₃-C₁₀ cycloalkyl, C₄-C₁₀ cycloalkenyl, C₆-C₁₀ aryl, C₆-C₁₀ aryl C₁-C₃ alkyl, C₆-C₁₀ heteroaryl and C₃-C₁₀ heterocyclic;
and a pharmaceutically acceptable carrier, excipient, or vehicle.

8. The pharmaceutical composition according to claim 1, wherein
one of R¹, R³, R⁴, R⁵, and R⁶ is C₁-C₆ alkyl, C₁-C₆ alkoxy, C₁-C₆ acyl, halo, oxo, ═NR⁷, —NHC(O)R⁷, —C(O)NH₂, —C(O)NHR⁷, —C(O)NR⁷R⁸, CO₂R⁷, or —SO₂R⁷, wherein R⁷R⁸ are as defined in claim 1; and
no more than four of the remainder of R¹, R², R³, R⁴, R⁵, and R⁶ are hydroxyl;
and a pharmaceutically acceptable carrier, excipient, or vehicle.

9. The pharmaceutical composition according to claim 1, wherein
two of R¹, R³, R⁴, R⁵, and R⁶ are C₁-C₆ alkyl, C₁-C₆ alkoxy, C₁-C₆ acyl, halo, oxo, ═NR⁷, —NHC(O)R⁷, —C(O)NH₂, —C(O)NHR⁷, —C(O)NR⁷R⁸, CO₂R⁷, or —SO₂R⁷, wherein R⁷R⁸ are as defined in claim 1; and
no more than three of R¹, R², R³, R⁴, R⁵, and R⁶ are hydroxyl;
and a pharmaceutically acceptable carrier, excipient, or vehicle.

10. The pharmaceutical composition according to claim 1, wherein
three of R¹, R³, R⁴, R⁵, and R⁶ are C₁-C₆ alky, C₁-C₆ alkoxy, C₁-C₆ alkyl, halo, oxo, ═NR⁷, —NHC(O)R⁷, —C(O)NH₂, —C(O)NHR⁷, —C(O)NR⁷R⁸, CO₂R⁷, or —SO₂R⁷, wherein R⁷R⁸ are as defined in claim 1; and
no more than two of R¹, R², R³, R⁴, R⁵, and R⁶ are hydroxyl;
and a pharmaceutically acceptable carrier, excipient, or vehicle.

11. The pharmaceutical composition according to claim 1, wherein four of R¹, R², R³, R⁴, R⁵, or R⁶ are hydroxyl; and
one of R¹, R², R³, R⁴, R⁵, or R⁶ is each independently selected from the group CH₃, OCH₃, NO₂, CF₃, OCF₃, F, Cl, Br, I and CN;
and a pharmaceutically acceptable carrier, excipient, or vehicle.

12. The pharmaceutical composition according to claim 1, wherein
five of R¹, R², R³, R⁴, R⁵, or R⁶ are hydroxyl; and
one of R¹, R², R³, R⁴, R⁵, or R⁶ is selected from CH₃, OCH₃, NO₂, CF₃, OCF₃, F, Cl, Br, I and CN;
and a pharmaceutically acceptable carrier, excipient, or vehicle.

13. The pharmaceutical composition according to claim 1, wherein
two, three, four or five of R¹, R², R³, R⁴, R⁵, or R⁶ are hydroxyl;
at least one of R¹, R², R³, R⁴, R⁵, or R⁶ is optionally substituted alkoxy; and
the remainder of R¹, R², R³, R⁴, R⁵, or R⁶ if any are independently selected from C₁-C₆ alkyl, C₂-C₆ alkenyl, C₂-C₆ alkynyl, C₁-C₆ alkoxy, C₂-C₆ alkenyloxy, C₃-C₁₀ cycloalkyl, C₁-C₆ acyl, C₁-C₆ acyloxy, hydroxyl, —NH₂, —NHR⁷, —NR⁷R⁸—, ═NR⁷, —S(O)₀₋₂R⁷, —SH, nitro, cyano, halo, haloalkyl, haloalkoxy, hydroxyalkyl, —CO₂R⁷, oxo, —PO₃H—NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆ alkenyl, C₂-C₆ alkynyl, C₃-C₁₀ cycloalkyl, C₄-C₁₀ cycloalkenyl, C₆-C₁₀ aryl, C₆-C₁₀ aryl C₁-C₃alkyl, C₆-C₁₀ heteroaryl and C₃-C₁₀ heterocyclic;
and a pharmaceutically acceptable carrier, excipient, or vehicle.

14. The pharmaceutical composition according to claim 13, wherein
five of R¹, R², R³, R⁴, R⁵, or R⁶ are hydroxyl; and
one of R¹, R², R³, R⁴, R⁵, or R⁶ is C₁-C₆ alkoxy;
and a pharmaceutically acceptable carrier, excipient, or vehicle.

15. The pharmaceutical composition according to claim 14, wherein at least one of R¹, R², R³, R⁴, R⁵, or R⁶ is methoxy;
and a pharmaceutically acceptable carrier, excipient, or vehicle.

16. The pharmaceutical composition according to claim 2, wherein
two, three, or four of R², R³, R⁴, R⁵, or R⁶ are hydroxyl;
R¹ is optionally substituted alkoxy; and
the remainder of R², R³, R⁴, R⁵, or R⁶ are independently selected from C₁-C₆ alkyl, C₂-C₆ alkenyl, C₂-C₆ alkynyl, C₁-C₆ alkoxy, C₂-C₆ alkenyloxy, C₃-C₁₀ cycloalkyl, C₁-C₆ acyl, C₁-C₆ acyloxy, hydroxyl, —NH₂, —NHR⁷, —NR⁷R⁸—, ═NR⁷, —S(O)₀₋₂R⁷, —SH, nitro, cyano, halo, haloalkyl, haloalkoxy, hydroxyalkyl, —CO₂R⁷, oxo, —PO₃H—NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆ alkenyl, C₂-C₆ alkynyl, C₃-C₁₀ cycloalkyl, C₄-C₁₀ cycloalkenyl, C₆-C₁₀ aryl, C₆-C₁₀ aryl C₁-C₃alkyl, C₆-C₁₀ heteroaryl and C₃-C₁₀ heterocyclic;
and a pharmaceutically acceptable carrier, excipient, or vehicle.

17. The pharmaceutical composition according to claim 16, wherein
R¹ is C₁-C₆ alkoxy; and
R², R³, R⁴, R⁵, and R⁶ are hydroxyl;
and a pharmaceutically acceptable carrier, excipient, or vehicle.

18. The pharmaceutical composition according to claim 17, wherein R¹ is methoxy;
and a pharmaceutically acceptable carrier, excipient, or vehicle.

19. A pharmaceutical composition wherein the compound is methyl-scyllo-inositol 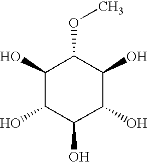
and a pharmaceutically acceptable carrier, excipient, or vehicle.

20. The pharmaceutical composition according to claim 1, wherein
two, three, four or five of R¹, R², R³, R⁴, R⁵, or R⁶ are hydroxyl;
at least one of R¹, R², R³, R⁴, R⁵, or R⁶ is halo; and
the remainder of R¹, R², R³, R⁴, R⁵, or R⁶, if any, are independently C₁-C₆ alkyl, C₂-C₆ alkenyl, C₂-C₆ alkynyl, C₁-C₆ alkoxy, C₂-C₆ alkenyloxy, C₃-C₁₀ cycloalkyl, C₁-C₆ acyl, C₁-C₆ acyloxy, —NH₂, —NHR⁷, —NR⁷R⁸—, ═NR⁷, —S(O)₀₋₂R⁷, —SH, nitro, cyano, halo, haloalkyl, haloalkoxy, hydroxyalkyl, —CO₂R⁷, oxo, —PO₃H—NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R³ are independently selected from C₁-C₆ alkyl, C₂-C₆ alkenyl, C₂-C₆ alkynyl, C₃-C₁₀ cycloalkyl, C₄-C₁₀ cycloalkenyl, C₆-C₁₀ aryl, C₆-C₁₀ aryl C₁-C₃alkyl, C₆-C₁₀ heteroaryl and C₃-C₁₀ heterocyclic;
and a pharmaceutically acceptable carrier, excipient, or vehicle.

21. The pharmaceutical composition according to claim 20, wherein
four of R¹, R², R³, R⁴, R⁵, or R⁶ are hydroxyl;
one of R¹, R², R³, R⁴, R⁵, or R⁶ is halo; and
one of R¹, R², R³, R⁴, R⁵, or R⁶ is selected from C₁-C₆ alkyl, C₂-C₆ alkenyl, C₂-C₆ alkynyl, C₁-C₆ alkoxy, C₂-C₆ alkenyloxy, C₃-C₁₀ cycloalkyl, C₁-C₆ acyl, C₁-C₆ acyloxy, hydroxyl, —NH₂, —NHR⁷, —NR⁷R⁸—, ═NR⁷, —S(O)OR⁷, —SH, nitro, cyano, halo, haloalkyl, haloalkoxy, hydroxyalkyl, —Si(R⁷)₃, —CO₂R⁷, oxo, —PO₃H—NHC(O)R⁷, —C(O)NH₂, —C(O)NHR⁷, —C(O)NR⁷R⁸, —NHS(O)₂R⁷, —S(O)₂NH₂, —S(O)₂NHR⁷, and —S(O)₂NR⁷R⁸ wherein R⁷ and R⁸ are independently selected from C₁-C₆ alkyl, C₂-C₆ alkenyl, C₂-C₆ alkynyl, C₃-C₁₀ cycloalkyl, C₄-C₁₀ cycloalkenyl, C₆-C₁₀ aryl, C₆-C₁₀ aryl C₁-C₃alkyl, C₆-C₁₀ heteroaryl and C₃-C₁₀ heterocyclic, and at least one of R¹, R², R³, R⁴, R⁵, or R⁶ is halo;
and a pharmaceutically acceptable carrier, excipient, or vehicle.

22. The pharmaceutical composition according to claim 1, wherein
five of R¹, R², R³, R⁴, R⁵, or R⁶ are hydroxyl; and
one of R¹, R², R³, R⁴, R⁵, or R⁶ is halo;
and a pharmaceutically acceptable carrier, excipient, or vehicle.

23. The pharmaceutical composition according to claim 2, wherein
R², R³, R⁴, R⁵, or R⁶ are hydroxyl, and
R¹ is halo;
and a pharmaceutically acceptable carrier, excipient, or vehicle.

24. The pharmaceutical composition according to claim 22, wherein halo is fluoro, chloro or bromo;
and a pharmaceutically acceptable carrier, excipient, or vehicle.

25. The pharmaceutical composition according to claim 23, wherein halo is fluoro, chloro or bromo;
and a pharmaceutically acceptable carrier, excipient, or vehicle.

26. A pharmaceutical composition wherein the compound is 1-chloro-1-deoxy-scyllo-inositol: 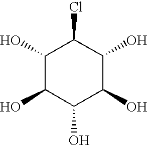
and a pharmaceutically acceptable carrier, excipient, or vehicle.

27. A pharmaceutical composition according to claim 1 for use in the treatment of a disease that is characterized by amyloid deposition.

28. A pharmaceutical composition according to claim 2 for use in the treatment of a disease that is characterized by amyloid deposition.

29. A pharmaceutical composition according to claim 1 wherein the disease is Alzheimer's disease.

30. A method for preventing, reducing and/or inhibiting in a subject Aβ fibril assembly or aggregation, Aβ toxicity, Aβ42 levels, abnormal protein folding or aggregation, amyloid formation, deposition, accumulation or persistence, and/or amyloid interactions comprising administering a therapeutically effective amount of the pharmaceutical composition of claim 1.

31. A method for increasing degradation of Aβ and/or reducing cerebral accumulation of amyloid β, deposition of cerebral amyloid plaques, soluble Aβ oligomers in the brain, glial activity, inflammation, and/or cognitive decline comprising administering a therapeutically effective amount of the pharmaceutical composition of as defined in claim 1.

32. A method for treating in a subject a condition of the central or peripheral nervous system or systemic organ associated with a disorder in protein folding or aggregation, or amyloid formation, deposition, accumulation, or persistence, comprising administering to the subject a therapeutically effective amount of a pharmaceutical composition as defined in claim 1.

33. A method for preventing or inhibiting amyloid protein assembly, enhancing clearance of amyloid deposits, or slowing deposition of amyloid deposits in a subject comprising administering to the subject a therapeutically effective amount of a pharmaceutical composition as defined in claim 1.

34. A method of delaying the progression of Alzheimer's disease in a subject comprising administering to the subject a therapeutically effective amount of a pharmaceutical composition as defined in claim 1.

35. A method for treating mild cognitive impairment (MCI) in a subject comprising administering to the subject a therapeutically effective amount of a pharmaceutical composition as defined in claim 1.

36. A regimen for supplementing a human's diet comprising administering a composition of the formula III as defined in claim 1 or a dietary supplement comprising a composition of the formula III as defined in claim 1, and an acceptable carrier, to the human.

37. The regimen of claim 36 wherein the administration is daily to the human.

38. The regimen of claim 37 wherein the composition of claim 1 is administered in an amount from about 5 milligrams to about 30 milligrams.

39. A kit comprising the composition of claim 1 containing at least one compound of formula III for preventing and/or treating a disease characterized by abnormal protein folding or aggregation or amyloid formation, deposition, accumulation or persistence, a container, and instructions for use.

40. The kit of claim 39 wherein the instructions provide information for treating Alzheimer's disease.
